(19) United States
(12) Patent Application Publication
(10) Pub. No.: US 2025/0387415 A1
(43) Pub. Date: Dec. 25, 2025

(54) COMPOSITIONS OF COPPER COMPLEXES

(71) Applicants: University of Lancashire, Preston, Lancashire (GB); Central University of Technology, Free State, Bloemfontein (ZA)

(72) Inventors: Chandrashekhar KULKARNI, Preston, Lancashire (GB); Sergey ZLATOGORSKY, Preston, Lancashire (GB); Pravin KENDREKAR, Preston, Lancashire (GB)

(73) Assignees: University of Lancashire, Preston, Lancashire (GB); Central University of Technology, Free State, Bloemfontein (ZA)

(21) Appl. No.: 19/110,053

(22) PCT Filed: Sep. 8, 2023
(86) PCT No.: PCT/GB2023/052334

§ 371 Date: Mar. 7, 2025

(30) Foreign Application Priority Data
Sep. 8, 2022 (GB) 2213123.9

(51) Int. Cl. A61K 31/616; A61K 47/14; A61K 47/30

(52) U.S. Cl. CPC A61K 31/616; A61K 47/14; A61K 47/30

(57) ABSTRACT

The present invention relates to compositions, in particular to lipid-based compositions that solubilise and/or encapsulate (sometimes high concentrations of) copper complex(es) (and/or derivatives or analogues thereof). Such lipid-based compositions offer a variety of uses, including in their capacity as topical or ingestible formulations. Such compositions permit delivery of high concentrations of copper complex(es) (and derivatives) in a highly bioavailable form.

### CROSS-REFERENCE TO RELATED APPLICATIONS

The present application is a U.S. National Stage Application under 35 U.S.C. § 371 of International Application No. PCT/GB2023/052334, filed on Sep. 8, 2023, which in turn claims the benefit of foreign priority under 35 U.S.C. § 119 from GB 2213123.9, filed on Sep. 8, 2022. The entire disclosure of the above applications is incorporated herein by reference.

### INTRODUCTION

The present invention relates to compositions, in particular to lipid-based compositions that solubilise and/or encapsulate (sometimes high concentrations of) a copper complex. The present invention also relates to methods of producing said compositions. The present invention also relates to various products that utilise or incorporate said compositions, and methods for making the same. Since the physical form of the compositions can be modulated, products containing or characterised by said compositions can enable a variety of modes of administration to and into the body. As such, the invention also pertains to various uses of said compositions and products, including medical uses.

### BACKGROUND

Copper, a trace element, is an essential component for the body¹. It has multifaceted biochemical roles, in particular, to bind and/or form complexes with ceruloplasmin, albumin, other proteins and nucleic acids¹. Copper performs a crucial role in the synthesis and stabilization of skin proteins. As much as 110 mg of copper is found in a human body of about 70 kg². It is vital to wound healing, as it promotes angiogenesis and skin ECM formation and stabilization². It also exhibits potent biocidal properties²: Several bacteria including antibiotic resistant bacteria as well as hard to kill bacterial spores, fungi and viruses are killed upon exposure to high concentrations of copper or copper compounds. Copper can have following oxidation states: metallic copper (Cu⁰), monovalent copper (Cuprous, Cu⁺) and divalent copper (cupric, Cu⁺⁺) ions. These ions are multifunctional in terms of their relevance in intracellular processes under normal as well as pathologic conditions. Copper used in dental amalgams and in the form of ointments is considered safe². Therefore, their therapeutic potential is getting recognized through further research advances in this direction.¹

Aspirin (FIG. 1e) is one of the most consumed drugs in the world; primarily used for relief of pain, inflammation and fever among several other medicinal applications³. It is a chemical derivative of salicylic acid (FIG. 1a) with anti-inflammatory properties. Although both of these compounds and their applicability were discovered over a century ago^{3,4} and are actively used even today, they have certain limitations and side-effects^{5,6}. Overuse or excessive dosage may lead to gastrointestinal bleeding and harm to patients undergoing surgical procedures⁷.

Around 1976, Sorensen^{8,9} demonstrated that copper complex of aspirin—copper acetyl salicylate (copper aspirinate, CuAsp) as per FIGS. 1(f) and (g)—is more potent as an anti-inflammatory agent and is less irritant to digestive track as compared to aspirin. The crystal structure of CuASP was determined even before these studies¹⁰. Subsequent studies further explored its applications in bio/medicinal areas including antiulcer, anticancer, antimutagenic, antithrombotic and antifungal activities^{1,8,9,11,12,13,14,15,16,17}. Bimetallic compounds of copper with ‘ibuprofen’ were also prepared which showed better activities as compared to native ibuprofen¹. However, CuASP and similar copper complex¹⁸ namely, Copper salicylate (CuSL) (FIGS. 1(b), (c), and (d)) are poorly soluble in water⁹.

These copper complexes make use of cupric cation (Cu²⁺) (FDA approved copper compounds in 1921) and aspirinato anion, parent compounds of which are approved by U.S. FDA a long ago (FDA approved Aspirin in 1899). Copper complexes have been previously administered via oral as well as parenteral routes^{9,14,19}, but they were used as solutions in organic solvents^{15,20} or aqueous suspensions¹⁹. For instance, in previous studies copper complexes were suspended in aqueous¹⁹ buffer (e.g. made of propelyene glycol or polyvinyl alcohol)²⁰ or dissolved in ethanol^{15,18}, methanol¹⁸, acetone¹⁸ or dimethyl sulfoxide (DMSO).^{14,18,20}

Although copper-drug complexes can offer major health benefits, their applicability is hampered due to innate concerns regarding low bioavailability, poor solubility and limited stability. Such problems are particularly pronounced for topical compositions, but also for oral dosage forms (including either solid or liquid forms, but especially solid).

It is an object of the present invention to provide alternative compositions, sometimes for alternative uses or in alternative products, to those described in the prior art.

It is an object of the present invention to provide compositions that permit relatively high loadings of copper complexes, especially where achievable without compromising bioavailability.

It is an object of the present invention to provide compositions that furnish copper-drug complexes in a bioavailable form, especially where achievable without having to reduce copper-drug complex loading.

It is an object of the present invention to provide lipid-based compositions the afford better copper complex solubility and/or better copper complex encapsulation.

It is an object of the present invention to provide lipid-based compositions from which copper complexes do not sediment after centrifugation—such compositions will suitable ensure copper complexes are well encapsulated and/or thoroughly dissolved within lipids, thus providing a more consistent product.

It is an object of the present invention to provide lipid-based composition that utilise as fewer ingredients (or ingredient types/categories) as possible, especially to avoid certain types of surfactants and polymeric materials.

It is an object of the present invention to provide lipid-based compositions that may be adapted for a variety of uses.

It is an object of the present invention to provide lipid-based compositions that are stable (especially physically stable) for long periods upon storage.

It is an object of the present invention to provide lipid-based compositions in which copper complexes remain chemically stable both during storage and during use (including upon the skin or within the body).

It is an object of the present invention to provide lipid-based compositions with high loadings of copper complexes that can be readily dispersed (suitably after being stored) in water or other solvents, so as to produce oil-in-water compositions. Suitably such compositions would be stored in the absent (or with only low levels) of any surfactants.

It is an object of the present invention to provide a lipid-based system which is particularly optimised for copper complex formulation, storage, and delivery (especially delivery through the skin or orally/gastrointestinally).

### REFERENCES

¹ Iakovidis, I.; Delimaris, I.; Piperakis, S. M., Copper and Its Complexes in Medicine: A Biochemical Approach. Molecular Biology International 2011, 2011, 594529
² Borkow, G., Using Copper to Improve the Well-Being of the Skin. Curr Chem Biol 2014, 8 (2), 89-102
³ Wu, K. K., Aspirin and Salicylate. Circulation 2000, 102 (17), 2022-2023
⁴ Sneader, W., The discovery of aspirin: a reappraisal. BMJ 2000, 321 (7276), 1591-1594
⁵ Ilowite, N. T.; Laxer, R. M., Chapter 6—PHARMACOLOGY AND DRUG THERAPY. In Textbook of Pediatric Rheumatology (Sixth Edition), Cassidy, J. T.; Laxer, R. M.; Petty, R. E.; Lindsley, C. B., Eds. W. B. Saunders: Philadelphia, 2011; pp 71-126
⁶ Cryer, B.; Mahaffey, K., Gastrointestinal ulcers, role of aspirin, and clinical outcomes: pathobiology, diagnosis, and treatment. Journal of Multidisciplinary Healthcare 2014, 7, 137-146
⁷ Kwok, C. S.; Loke, Y. K., Critical Overview on the Benefits and Harms of Aspirin. Pharmaceuticals (Basel) 2010, 3 (5), 1491-1506
⁸ Sorenson, J. R. J., Copper chelates as possible active forms of the antiarthritic agents. Journal of Medicinal Chemistry 1976, 19 (1), 135-148
⁹ RAINSFORD, K. D.; WHITEHOUSE, M. W., Concerning the merits of copper aspirin as a potential anti-inflammatory drug. Journal of Pharmacy and Pharmacology 1976, 28 (1), 83-86
¹⁰ Manojlović-Muir, L., The crystal structure of copper(II) aspirinate. Acta Crystallographica Section B 1973, 29 (10), 2033-2037
¹¹ Alich, A. A.; Welsh, V. J.; Wittmers, L. E., Comparison of Aspirin and Copper Aspirinate with Respect to Gastric Mucosal Damage in the Rat. J Pharm Sci-Us 1983, 72 (12), 1457-1461
¹² SHEN, Z. Q.; LIANG, Y.; CHEN, Z. H.; LIU, W. P.; DUAN, L., Pharmacology: Effects of Copper-aspirin Complex on Platelet Aggregation and Thrombosis in Rabbits and Mice. Journal of Pharmacy and Pharmacology 1998, 50 (11), 1275-1279
¹³ Zhiqiang, S.; Lei, W. Y.; Li, L.; Chen, Z. H.; Liu, W. P., Coordination of copper with aspirin improves its anti-inflammatory activity. Inflammopharmacology 1998, 6 (4), 357-62
¹⁴ Fujimori, T.; Yamada, S.; Yasui, H.; Sakurai, H.; In, Y.; Ishida, T., Orally active antioxidative copper(II) aspirinate: Synthesis, structure characterization, superoxide scavenging activity, and in vitro and in vivo antioxidative evaluations. Journal of biological inorganic chemistry: JBIC: a publication of the Society of Biological Inorganic Chemistry 2006, 10, 831-41
¹⁵ Ogundeji, A. O.; Porotloane, B. F.; Pohl, C. H.; Kendrekar, P. S.; Sebolai, O. M., Copper Acyl Salicylate Has Potential as an Anti-Cryptococcus Antifungal Agent. Antimicrob Agents Chemother 2018, 62 (8)
¹⁶ Sorenson, J. R. J., Copper complexes in the treatment of experimental inflammatory conditions: inflammation, ulcers and pain. In Copper and Zinc in Inflammation, Milanino, R.; Rainsford, K. D.; Velo, G. P., Eds. Springer Netherlands: Dordrecht, 1989; pp 69-84
¹⁷ Weder, J. E.; Dillon, C. T.; Hambley, T. W.; Kennedy, B. J.; Lay, P. A.; Biffin, J. R.; Regtop, H. L.; Davies, N. M., Copper complexes of non-steroidal anti-inflammatory drugs: an opportunity yet to be realized. Coordination Chemistry Reviews 2002, 232 (1), 95-126
¹⁸ Ogodo, U. P.; Abosede, O. O., Synthesis and characterization of Cu (II) complexes of salicylate ligands Journal of Applied Sciences and Environmental Management 2018, 22 (12), 1961-1964
¹⁹ Chohan, Z. H.; Iqbal, M. S.; Iqbal, H. S.; Scozzafava, A.; Supuran, C. T., Transition metal acetylsalicylates and their anti-inflammatory activity. J Enzyme Inhib Med Chem 2002, 17 (2), 87-91
²⁰ Liu, W.; Xiong, H.; Yang, Y.; Li, L.; Shen, Z.; Chen, Z., Potential application of copper aspirinate in preventing and treating thromboembolic diseases. Met Based Drugs 1998, 5 (3), 123-126

### SUMMARY OF THE INVENTION

According to an aspect of the present invention, there is provided a composition comprising a metal complex and one or more glycerides, wherein at least one of the glyceride(s) comprises at least one unsaturated moiety, preferably at least one alkene moiety, most preferably at least one cis-alkene moiety.

According to an aspect of the present invention, there is provided a composition comprising a metal compound and one or more glycerides, wherein at least one of the glyceride(s) comprises at least one unsaturated moiety, preferably at least one alkene moiety, most preferably at least one cis-alkene moiety.

According to an aspect of the present invention, there is provided a composition comprising a metal compound and/or metal complex (or salt thereof); and one or more glycerides, wherein at least one of the glyceride(s) comprises at least one unsaturated moiety, preferably at least one alkene moiety, most preferably at least one cis-alkene moiety.

According to an aspect of the present invention, there is provided a composition, suitably a composition comprising a copper complex. The copper complex is suitably a copper-drug complex. As indicated below, the term “copper complex” (or similar terms, such as “copper-drug complex”) may be directly substituted by “a copper compound comprising a copper complex and/or a (suitably pharmaceutically-acceptable) salt thereof”. Suitably, references to a “copper complex” (or similar term) includes any counterions that may be associated therewith. The composition is suitably a lipid-based composition comprising a copper complex and one or more lipids. Suitably the one or more lipids are one or more glycerides. As such, suitably the composition comprises one or more glycerides, suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

According to an aspect of the present invention, there is provided a composition comprising a metal-drug complex (suitably a copper-drug complex) and one or more glycerides, wherein the metal-drug complex comprises one or metal atoms and/or metal ions (suitably one or more copper atoms and/or copper ions, most preferably one or more copper(II) ions) co-ordinated with one or more drug molecules/ligands (or ionised forms of said drug molecule(s)/ligand(s)), suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

According to an aspect of the present invention, there is provided a composition comprising a copper-drug complex and one or more glycerides, suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

According to an aspect of the present invention, there is provided a composition comprising:
a copper-drug complex (or salt thereof) comprising one or more copper ion(s), and one or more ligand(s) datively bonded to at least one (of the) copper ion(s), wherein at least one of the ligand(s) is a drug molecule (or ionised form thereof); and
one or more glycerides, wherein at least one of the glyceride(s) comprises at least one unsaturated moiety, preferably at least one alkene moiety, most preferably at least one cis-alkene moiety.

According to an aspect of the present invention, there is provided a composition comprising either or both copper aspirinate and/or copper salicylate (or relevant salts, complexes, or derivatives of either or both), suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

In respect of the aforesaid aspects, suitably the or each glyceride (suitably each mono- and/or diglyceride) comprises one, two, or three fatty acid(s) (suitably respectively comprises one or two fatty acids in the case of mono- and/or diglyceride) condensed with glycerol. Suitably, the or at least one of, most suitably each (or all) of, said fatty acid(s) are C₆-C₃₀ fatty acid(s) (i.e. having a carbon chain that is 6-30 carbons in length, including the carboxylate carbon), preferably C₈-C₂₀ fatty acid(s), most preferably C₁₈ fatty acid(s). Preferably at least one (preferably each) of the fatty acid(s) comprise at least one unsaturated moiety, preferably at least one alkene moiety, most preferably at least one cis-alkene moiety. Most preferably, the fatty acid(s) is or comprises oleic acid, and most preferably the glyceride is (or the glycerides comprise) glyceryl monooleate (monoolein). In alternative embodiments, the fatty acid(s) is or comprises linoleic acid (suitably in addition to or instead of oleic acid), in which case most preferably the glyceride is (or the glycerides comprise) glyceryl monolinoleate (monolinolein) (suitably in addition to or instead of glyceryl monooleate).

According to an aspect of the present invention, there is provided a method of preparing a composition (suitably as defined herein), said method being suitably as defined herein. The method suitably comprises mixing a copper complex with the one or more glycerides.

According to an aspect of the present invention, there is provided a composition obtained by (or directly obtained by) the aforesaid method of preparing a composition.

According to an aspect of the present invention, there is provided a product comprising a composition (suitably as defined herein), said product being suitably as defined herein.

According to an aspect of the present invention, there is provided a method of preparing a product (suitably as defined herein), said method being suitably as defined herein. The method suitably comprises incorporating the composition within the product, suitably by mixing the composition with other components/ingredients of the product.

According to an aspect of the present invention, there is provided a product obtained by (or directly obtained by) the aforesaid method of preparing a product.

According to an aspect of the present invention, there is provided a composition for use as a medicament (or for use in treating a disease, disorder, or medical condition, suitably a disease, disorder, or medical condition as defined herein).

According to an aspect of the present invention, there is provided a use of a composition (suitably as defined herein, e.g. a pharmaceutical composition) or a product (suitably as defined herein, e.g. a pharmaceutical product) in the manufacture of a medicament (or in the manufacture of a medicament for treating a disease, disorder, or medical condition, suitably a disease, disorder, or medical condition as defined herein).

According to an aspect of the present invention, there is provided a method of treating a disease, disorder, or medical condition (suitably a disease, disorder, or medical condition as defined herein) in a subject (suitably in a subject identified as in need of treatment), the method comprising administering to the subject a therapeutically effective amount of a composition (suitably as defined herein, e.g. a pharmaceutical composition) or a product (suitably as defined herein, e.g. pharmaceutical product).

Herein, references to a “copper complex” (general or specific) may be substituted by the term “metal complex”, “metal-drug complex”, “aqueous-insoluble metal complex”, “aqueous-insoluble metal-drug complex”, “transition metal complex”, “transition metal-drug complex”, “aqueous-insoluble transition metal complex”, or “aqueous-insoluble transition metal-drug complex”, since the principles of the present invention may be applied more broadly. By the same token, references herein to copper aspirinate may be more generally applicable to any metal aspirinate, especially any aqueous-insoluble metal aspirinate. The same applies to copper salicylate—it may be substituted by “metal salicylate” or “aqueous-insoluble metal salicylate”.

Any features, including optional, suitable, and preferred features, described in relation to any particular aspect of the invention may also be features, including optional, suitable and preferred features, of any other aspect of the present invention unless incompatible therewith.

### BRIEF DESCRIPTION OF THE DRAWINGS

For a better understanding of the invention, and to show how embodiments of the same are put into effect, reference is now made, by way of example, to the following diagrammatic drawings, in which:

FIG. 1 shows chemical structures for: a) salicylic acid; b) copper(II) salicylate (possible structure 1); c) copper(II) salicylate (possible structure 2); d) copper(II) salicylate (possible structure 3); e) aspirin (acetyl salicylic acid); f)) copper(II) acetylsalicylate (copper aspirinate) (possible structure 1); and g) copper(II) acetylsalicylate (copper aspirinate) (possible structure 2). Here b) and f) are mononuclear complexes whereas c), d) and g) are binuclear complexes.

FIG. 2 shows an image of a formulation containing CuASP solubilized in molten MO.

FIG. 3 shows is a trace showing small angle X-ray scattering patterns of pure lipid and CuASP-loaded lipid mixture (CuASP solubilized in molten MO+water). Points 1,2,3 . . . display characteristic peaks for lamellar phase for monoolein at room temperature.

FIG. 4 shows two images of a formulation containing CuASP encapsulated in DU (F01-CuASP-DU-E) at room temperature (Left) and molten state (Right).

FIG. 5 shows an image of a formulation containing CuSL solubilized in molten DU (F01-CuSL-DU).

FIG. 6 shows two images of a formulation containing CuSL encapsulated in DU (F01-CuSL-DU-E) at room temperature (Left) and molten state (Right).

FIG. 7 shows an image of a formulation containing CuASP solubilized in molten MO+water.

FIG. 8 shows a trace showing small angle X-ray scattering patterns of pure lipid in water and CuASP loaded lipid-water mixture. Points √2, √3, √4 . . . display characteristic peaks for Pn3m cubic phase for monoolein in excess water at room temperature.

FIG. 9 shows nine images of 1 mm-thick transparent glass slides containing, within each's central well, a formulation containing 0.75 g dimodan (DU), 0.5 g water, and the following various amounts of copper(II) aspirinate: a) 10 mg; b) 15 mg; c) 20 mg; d) 40 mg; e) 60 mg; f) 70 mg; g) 80 mg; h) 90 mg; i) 100 mg.

FIG. 10 shows various hydrated copper aspirinate/dimodan (DU) formulations (F02-CuASP-DU and F02-CuASP-DU-E) wherein copper aspirinate is solubilised and, for higher concentrations, is encapsulated. Characteristic turquoise colour is clearly visible from CuASP in lipid cubic phases. The numbers in mg indicate CuASP content in 1.25 g cubic phases (formed of 0.75 g lipid+0.5 g water).

FIG. 11 shows four images of 1 mm-thick transparent glass slides containing, within each's central well, a formulation containing 0.75 g dimodan (DU), 0.5 g water, and the following various amounts of copper(II) salicylate: a) 10 mg; b) 15 mg; c) 29 mg; d) 30 mg.

FIG. 12 shows images of various hydrated copper salicylate/dimodan (DU) formulations (F02-CuSL-DU and F02-CuSL-DU-E) wherein copper salicylate is solubilised and, for higher concentrations, is encapsulated. Characteristic green colour is clearly visible from CuSL in lipid cubic phases. The numbers in mg indicate CuASP content in 1.25 g cubic phases (formed of 0.75 g lipid+0.5 g water).

FIG. 13 shows images of various oil-in-water emulsions containing CuASP/dimodan (DU). The numbers in mg indicate CuASP content in 10 g cubosomes solution (formed of 0.5 g lipid+9.5 g of 0.5% F127 solution in water). 26 mg CuASP was sedimented, so the solubility limit was reached at 25 mg CuASP in 10 g cubosomes (which is equal to 50 mg/g of lipid).

FIG. 14 shows images of various oil-in-water emulsions containing CuSL/dimodan (DU). The numbers in mg indicate CuSL content in 10 g cubosomes solution (formed of 0.5 g lipid+9.5 g of 0.5% F127 solution in water). 5 mg CuSL was sedimented, so the solubility limit was reached at 4 mg CuSL in 10 g cubosomes (which is equal to 8 mg/g of lipid).

FIG. 15 shows images of three oil-in-water emulsions containing CuASP in DU and various aqueous solutions of pluronic F127: a) 5 mg CuASP, 2 g dimodan (DU), 8 g of 2 wt % F127 solution; b) 5 mg CuASP, 4 g dimodan (DU), 6 g of 5 wt % F127 solution; c) 5 mg CuASP, 5 g dimodan (DU), 5 g of 5 wt % F127 solution.

FIG. 16 shows images of four oil-in-water emulsion containing CuASP in DU+coconut oil and various aqueous solutions of pluronic F127: a) 5 mg CuASP, 0.9 g dimodan (DU), 0.1 g coconut oil, 9 g of 0.5 wt % F127 solution; b) 5 mg CuASP, 1.8 g dimodan (DU), 0.2 g coconut oil, 8 g of 0.5 wt % F127 solution; c) 5 mg CuASP, 3.6 g dimodan (DU), 0.4 g coconut oil, 6 g of 1 wt % F127 solution; a) 5 mg CuASP, 4.5 g dimodan (DU), 0.5 g coconut oil, 5 g of 2 wt % F127 solution.

FIG. 17 shows images of five oil-in-water emulsions containing CuSL in DU and various aqueous solutions of pluronic F127: a) 5 mg CuSL, 0.5 g dimodan (DU), 9.5 g of 0.5 wt % F127 solution; b) 5 mg CuSL, 1 g dimodan (DU), 9 g of 1 wt % F127 solution; c) 5 mg CuSL, 2 g dimodan (DU), 8 g of 2 wt % F127 solution; d) 5 mg CuSL, 4 g dimodan (DU), 6 g of 5 wt % F127 solution; e) 5 mg CuSL, 5 g dimodan (DU), 5 g of 5 wt % F127 solution.

FIG. 18 shows images of four oil-in-water emulsion containing CuSL in DU+coconut oil and various aqueous solutions of pluronic F127: a) 5 mg CuSL, 0.9 g dimodan (DU), 0.1 g coconut oil, 9 g of 0.5 wt % F127 solution; b) 5 mg CuSL, 1.8 g dimodan (DU), 0.2 g coconut oil, 8 g of 0.5 wt % F127 solution; c) 5 mg CuSL, 3.6 g dimodan (DU), 0.4 g coconut oil, 6 g of 1 wt % F127 solution; a) 5 mg CuSL, 4.5 g dimodan (DU), 0.5 g coconut oil, 5 g of 2 wt % F127 solution.

### DETAILED DESCRIPTION OF THE INVENTION

### Definitions

Unless otherwise stated, the following terms used in the specification and claims have the following meanings set out below.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

Unless stated otherwise, any reference herein to an “average” value is intended to relate to the mean value.

Unless otherwise stated, wherever a claim or paragraph refers to another claim/paragraph “or any other claim/paragraph dependent thereon” (or similar such language), such “other claim/paragraph dependent thereon” includes other claims/paragraphs that are directly or indirectly (i.e. via one or more other dependencies) dependent thereon.

Herein, the term “compound of Formula [X]”, where [X] is a number, typically a roman numeral optionally followed by an alphanumeric character, may be abbreviated to “compound [X]”. A reference to either a “compound of Formula [X]” or “compound [X]” suitably includes a salt (e.g. pharmaceutically acceptable salt), ionised form (e.g. carboxylate where the compound or molecule contains a carboxylic acid moiety), or solvate (e.g. hydrate) thereof, and suitably also includes a synthetic equivalent thereof.

A “composition” typically comprises components and ingredients. Ingredients may also be referred to as components, and may be used interchangeably, especially in respect of compositions.

The term “ingredient” (or component) includes general ingredients (e.g. defined by reference to an ingredient class, for example, a sugar) or specific ingredients (e.g. sucrose, which is a specific ingredient from the sugar ingredient class).

Although not intended to be limiting, an ingredient may be defined by reference to a label, a name (especially where said name is well-known), a structure, a function, any other appropriate means, or any combination thereof. Where an ingredient (especially an ingredient class) is referred to by way of a functional label (e.g. a label or name which mentions a function or uses functional language, for example a “sugar stabiliser”), said ingredient may serve the stated function (e.g. in the context of a given composition), especially where said stated function can be readily verified by means stated within this specification. However, said functional label may be simply a label that demands no particular verification of the stated function, especially where this specification provides no particularly methods of verifying said function—in such cases the skilled person will readily discern the scope of the functionally-labelled ingredient by reference to other non-functional parts of the functional label and/or by reference to the context in which said functional label is applied, optionally by reference to any sub-definitions or definitions of other ingredients (or ingredient classes). As such, the term “sugar stabiliser” may simply mean a sugar, suitably a sugar as further defined by way of sub-definitions.

An ingredient (or component) may in itself be composed of (or comprise) a plurality of ingredients (e.g. sub-ingredients). For example, a lipid component may, unless stipulated to the contrary (e.g. “the lipid component is [a single lipid]”, or “the lipid component consists of [a specific lipid]”), comprise more than one lipid. As such, any general principles defined herein in respect of a composition (e.g. amounts; the notion of a composition consisting essentially of one or more components—this may equally apply to a component with sub-components/sub-ingredients) may also be applicable to a component capable itself of comprising a plurality of sub-components.

Where a composition is said to comprise a plurality of stipulated ingredients (optionally in stipulated amounts of concentrations), said composition may optionally include additional ingredients other than those stipulated. However, a composition said to comprise a plurality of stipulated ingredients may in fact consist essentially of or consist of all the stipulated ingredients, optionally in the amounts specified, optionally (where compatible with the applicable context) with a solvent system (e.g. water). In either circumstance, an individual component may itself comprise, consist essentially of, or consist of a sub-component or one or more sub-components. Herein, wherever the term “comprise” is used it may (whether in the context of a composition or component/ingredient thereof), where compatible within the applicable context, be replaced by “consists essentially of” or “consists of”.

Herein, unless incompatible with the context or unless stated otherwise, the term “stipulated” used in the context of a “stipulated [ingredient/component]” preferably means ingredients recited as being present within any given composition (or component).

Amounts given herein in respect of any given ingredient class will, unless specified to the contrary, be applicable to any specific ingredient within said ingredient class.

Herein, amounts stipulated for components and ingredients, whether specified in terms of “parts”, ppm (parts per million), percentages (%, e.g. wt %), or ratios, are intended to be by weight, unless stated otherwise.

“About” when used to modify a numerically defined parameter (e.g., a pH value) means that the parameter may vary, e.g., within the experimental accuracy of determining the parameter or by as much as much as 5% below or above the stated numerical value for that parameter, preferably by as much as 2% below or above the stated numerical value for that parameter. In preferred embodiments, a parameter described by the term “about” corresponds to the stated numerical value.

Herein, where a composition (or indeed a component that may contain sub-components) is said to “consists essentially of” particular component(s), said composition suitably comprises at least 70 wt % of said component(s), suitably at least 90 wt % thereof, suitably at least 95 wt % thereof, most suitably at least 99 wt % thereof. Suitably, a composition said to “consist essentially of” particular component(s) consists of said component(s) save for one or more trace impurities.

Where the quantity or concentration of a particular component of a given composition is specified as a weight percentage (wt % or % w/w), said weight percentage refers to the percentage of said component by weight relative to the total weight of the composition as a whole. It will be understood by those skilled in the art that the sum of weight percentages of all components of a composition will total 100 wt %. However, where not all components are listed (e.g. where compositions are said to “comprise” one or more particular components), the weight percentage balance may optionally be made up to 100 wt % by unspecified ingredients (e.g. a diluent, such as water, or other non-essentially but suitable additives). Most suitably, the sum of wt % of stipulated ingredients does not exceed 100 wt % and any combinations of wt % that would do so would by definition be excluded.

Herein, unless stated otherwise, the term “parts” (e.g. parts by weight, pbw) when used in relation to multiple ingredients/components, refers to relative ratios between said multiple ingredients/components. Expressing molar or weight ratios of two, three or more components gives rise to the same effect (e.g. a molar ratio of x, y, and z is x1: y1: z1 respectively, or a range x1-x2: y1-y2: z1-z2). Though in many embodiments the amounts of individual components within a composition may be given as a “wt %” value, in alternative embodiments any or all such wt % values may be converted to parts by weight (or relative ratios) to define a multi-component composition. This is so because the relative ratios between components is often more important than the absolute concentrations thereof in the liquid pharmaceutical compositions of the invention. Where a composition comprising multiple ingredients is described in terms of parts by weight alone (i.e. to indicate only relative ratios of ingredients), it is not necessary to stipulate the absolute amounts or concentrations of said ingredients (whether in toto or individually) because the advantages of the invention can stem from the relative ratios of the respective ingredients rather than their absolute quantities or concentrations. However, in certain embodiments, such compositions consists essentially of or consist of the stipulated ingredients and a diluents (e.g. water).

The term “mole percent” (i.e. mol %) is well understood by those skilled in the art, and the mol % of a particular constituent means the amount of the particular constituent (expressed in moles) divided by the total amount of all constituents (including the particular constituent) converted into a percentage (i.e. by multiplying by 100). The concept of mol % is directly related to mole fraction.

The term “substantially free”, when used in relation to a given component of a composition (e.g. “a composition substantially free of compound X”), refers to a composition to which essentially none of said component has been added. When a composition is “substantially free” of a given component, said composition suitably comprises no more than 1 wt %, suitably not more than 0.1 wt %, suitably no more than 0.01 wt %, suitably no more than 0.001 wt % of said component, suitably no more than 0.0001 wt % of said component, suitably no more than 0.00001 wt %, suitably no more than 0.000001 wt %, suitably no more than 0.0000001 wt % thereof, most suitably no more than 0.0001 parts per billion (by weight).

The term “free” or “entirely free”, when used in relation to a given component of a composition (e.g. “a composition entirely free of compound X”), refers to a composition containing none of said component.

Herein, unless incompatible in a given context, wherever a component is stipulated which is capable of ionization (e.g. protonation or deprotonation), the definition of said component preferably includes any suitable salts thereof, preferably pharmaceutically acceptable salts thereof. For example, this applies to any references herein to buffering agents (e.g. citric acid or citrate), amino acids, and such like. Likewise, unless incompatible in a given context, wherever a component is stipulated which is capable of neutralisation, the definition of said component preferably includes neutralised forms thereof
e.g. citric acid instead of citrate.

Herein, in the context of the present specification, a “strong acid” is suitably one having a pKa of −1.0 or less, whereas a “weak acid” is suitably one having a pKa of 2.0 or more. Herein, in the context of the present specification, a “strong base” is suitably one whose conjugate acid has a pKa of 12 or higher (suitably 14 or higher), whereas a “weak base” is suitably one whose conjugate acid has a pKa of 10 or less.

Unless stated otherwise, references herein to a “pKa” should be construed as a pKa value in water at standard ambient temperature and pressure (SATP), suitably of the conjugate acid of the relevant species.

Suitably, unless stated otherwise, where reference is made to a parameter (e.g. pH, pKa, etc.) or state of a material (e.g. liquid, gas, etc.) which may depend on pressure and/or temperature, suitably in the absence of further clarification such a reference refers to said parameter at standard ambient temperature and pressure (SATP). SATP is a temperature of 298.15 K (25° C., 77° F.) and an absolute pressure of 100 kPa (14.504 psi, 0.987 atm).

The invention concerns amongst other things the treatment of conditions treatable by metal complexes (especially copper-drug complexes such as copper aspirinate). The term “treatment”, and the therapies encompassed by this invention, include the following and combinations thereof: (1) inhibiting, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment”, “prophylaxis” and “inhibitor” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “inhibit” includes reference to delaying, stopping, reducing the incidence of, reducing the risk of and/or reducing the severity of an event, state, disorder or condition. Inhibiting an event, state, disorder or condition may therefore include delaying or stopping initiation and/or progression of such, and reducing the risk of such occurring. The products/compositions of the disclosure may be used to inhibit events, disorders and/or conditions which are disclosed herein.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

Routes of administration useful in the disclosed methods include but are not limited to topical, oral, parenteral intravenous, intraperitoneal (ip), rectal, ophthalmic, nasal, and transdermal.

Herein, unless stated otherwise, all chemical nomenclature may be defined in accordance with IUPAC definitions.

Herein, the term “hydrocarbon” is well understood in the art, and refers to compounds containing carbon and hydrogen only. The term “hydrocarbyl” general refers any aliphatic, acyclic, or cyclic (including aryl) hydrocarbon group, suitably with no heteroatoms. It may also encompass polycyclic groups, including groups with fused rings. Such compounds include, inter alia, alkanes, alkenes, alkynes, arenes, and cyclic versions thereof. The term “hydrocarbon” anthracene, naphthalene, benzene, and/or derivatives thereof (e.g. toluene).

Herein, the term “carbocyclyl”, “carbocycle” or “carbocyclic” refers to a radical of a non-aromatic cyclic hydrocarbon group, generally having from 3 to 10 ring carbon atoms (i.e. (3-10C)carbocyclyl) and zero heteroatoms in the non-aromatic ring system. Suitably, carbocyclyl groups include (3-nC)cycloalkyl and (3-nC)cycloalkenyl. Exemplary embodiments include: cyclobutyl, cyclobutenyl, cyclopentyl, cyclopentenyl, cyclohexyl, cyclohexenyl, cyclohexadienyl, cycloheptyl, cycloheptenyl, cycloheptadienyl, cycloheptatrienyl, cyclooctyl, cyclooctenyl, bicyclo[2.2.1]heptanyl, bicyclo[2.2.2]octanyl, and the like.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO2 groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered heteroaromatic ring containing 1, 2 or 3 ring heteroatoms; and
o) a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

Wherever groups with large carbon chains are disclosed (e.g. (1-12C)alkyl, (1-8C)alkenyl, etc.), such groups may optionally be shortened, for instance containing a between 1 and 5 carbons (e.g. (1-5C)alkyl or (1-5C)alkenyl), or contain between 1 and 3 carbons (e.g. (1-3C)alkyl or (1-3C)alkenyl instead of (1-12C)alkyl or (1-8C)alkenyl).

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess telomerase inhibitory activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the formula I may exhibit polymorphism, and that the invention encompasses all such forms.

Compounds may exist in a number of different tautomeric forms and references to compounds include all such forms. For the avoidance of doubt, where a compound can exist in one of several tautomeric forms, and only one is specifically described or shown, all others are nevertheless embraced by the definition of the compound. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

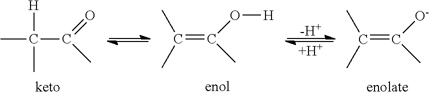

Herein, the term “particle size” or “pore size” refers respectively to the length of the longest dimension of a given particle or pore. Particle and pore sizes may be measured using methods well known in the art, including a laser particle size analyser and/or electron microscopes (e.g. transmission electron microscope, TEM, or scanning electron microscope, SEM).

The term “lipid” is well understood by a person skilled in the art, and generally hydrophobic or amphiphilic compounds and may inter alia include fats (incl. fatty acids, fatty acid esters, glycerides, fatty alcohols), waxes, sterols, fat-soluble vitamins (such as vitamins A, D, E, and K), monoglycerides, diglycerides, triglycerides, and phospholipids. Taking account of the biosynthesis of lipids and building blocks for their generation, e.g. ketoacyl or isoprene moieties, lipids may be categorised as fatty acids, glycerolipids, glycerophospholipids, sphingolipids, saccharolipids, and polyketides (the aforementioned being derivable through condensation of ketoacyl subunits); and sterol lipids and prenol lipids (which are derivable through condensation of isoprene subunits). The term lipid includes “glycerides” defined herein in relation to the invention. As such, references herein to lipids other than glycerides (e.g. in general or other than specific glycerides) generally refers to all of the above other than glycerides or the specific glycerides stipulated. Preferably, the term lipids encompasses only compounds comprising a fatty acid moiety (incl. fatty acids, fatty acid esters, glycerides, fatty alcohols), and, as such, lipids excluding glycerides (or specified glycerides) are compounds comprising a fatty acid moiety except for glycerides (or specified glycerides).

The term “oil” is well understood by a person skilled in the art, and refers to a hydrophobic, lipophilic, viscous liquid (at SATP) which generally comprises a mixture of compounds, suitably predominantly (50%, >60%, >70%, >80%, >90%, most suitably 95%) hydrophobic and lipophilic compound(s), suitably at least two or more of such compounds, suitably at least three or more such compounds, suitably at least four or more such compounds. Suitably all of the compound(s) present within any given oil are neutral (i.e. non-ionic and/or non-ionised). The or each oil suitably has a partition coefficient (octanol-Water partition coefficients, Log P) of at least 3, suitably at least 4, suitably at least 4.5, suitably at least 5, suitably at least 6. Suitably at least 70 wt % of the total weight of the or each oil comprises compounds which have a partition coefficient (octanol-Water partition coefficients, Log P) of at least 3, suitably at least 4, suitably at least 4.5, suitably at least 5, suitably at least 6, suitably at least 80 wt %, suitably at least 90 wt %, suitably at least 95 wt %. An “oil” tends to have a high carbon and high hydrogen content and are typically flammable. An “oil” is suitably surface active. An oil suitably has a particular viscosity measurable by techniques well known in the art. Herein, unless stated otherwise, all viscosity quantities given herein are kinematic viscosities at SATP, which is determinable by obtaining a dynamic viscosity (suitably at SATP, suitably at shear rates ranging from 0.1 sec⁻¹ to 100 sec⁻¹ or taking a dynamic viscosity as an average in these shear rate ranges) and converting to a kinematic viscosity in accordance with the following equation:

$$ν = μ / ρ \qquad ( 1 )$$

where v is kinematic viscosity, μ is dynamic viscosity, and ρ is density of the overall fluid. Kinematic viscosities are expressed in centistokes (cSt), though SI units for kinematic viscosity are m²/s. Water at 20° C. has a kinematic viscosity of approximately 10⁻⁶ m²·s⁻¹ or 1 cSt. Suitably, the oil component has a kinematic viscosity between 10 and 500 cSt at SATP, suitably between 20 and 250 cSt, suitably between 30 and 150 cSt, suitably between 40 and 100 cSt, suitably between 50 and 80 cSt at SATP. Oils suitably comprise lipids (suitably at least 90 wt % lipids, suitably at least 95 wt % lipids, suitably at least 99 wt % lipids). Suitably, any amounts of oils is in addition to any amounts of stipulated components (e.g. specific glycerides) that may happen also to be present within the oils as well. Organic oils are generally derived from animal sources, plant sources, and other organisms—e.g. animal oils, vegetable oils, essential oils (which are generally from plants). Such organic oils are often produced through natural metabolic processes within such organisms. Where organic oils are used in the context of the present invention, suitably such organic oils exclude plant-derived essential oils. Organic oils generally comprise a mixture of lipids, and may optionally further include one or more substances selected from the group consisting of proteins, waxes, and alkaloids. The mixture of lipids may comprise compounds in one or more compound classes selected from the group consisting of fatty acids, glycerolipids, sterol lipids, steroids, phospholipids, sphingolipids, glycerophospholipids, prenol lipids, saccharolipids, polyketides, and optionally esters of any of the aforementioned.

References to fatty acids of glycerides refers to the fatty acyl moiety (i.e. the fatty acid that is condensed to the hydroxyl of glycerol).

The term “copper complex”, which is a particular metal complex (or co-ordination complex), is well understood in the art. A copper complex comprises at least one copper atom or ion (typically copper(I) or copper(II), most preferably copper(II)) that is co-ordinated (suitably via dative bonding) to one or more ligands—when two or more ligands are present, they may be the same or different to each other. Such ligand(s) may be charged (if so, most suitably negatively charged) or neutral. Such ligand(s) suitably comprise one or more lone-pair(s) of electrons that co-ordinate to, or are capable of co-ordinating to, the at least one copper atom or ion.

Unless specified otherwise, or incompatible in the given context, references herein to a “copper complex” (or any more-specifically defined copper complex) includes any salts (suitably pharmaceutically acceptable salts) thereof. As such, references herein to a copper complex (be it in general or more-specifically defined, e.g. copper aspirinate) may be substituted by the term “copper compound” or “copper complex compound”, where said copper compound comprises or consists of the copper complex or a (preferable pharmaceutically-acceptable) salt thereof. A salt of a copper complex may, for instance, comprise or consist of a charged copper co-ordination complex (which may be charged by virtue of the prevailing balance of charges of metal(s) and ligand(s)) and one or more relevant counterions. Alternatively or additionally, such salts may be a consequence of salt forms of a ligand—for instance, a salt of a ligand's amine group (e.g. hydrochloride salt) or a salt of a ligand's carboxylic acid group (e.g. sodium salt a the corresponding carboxylate).

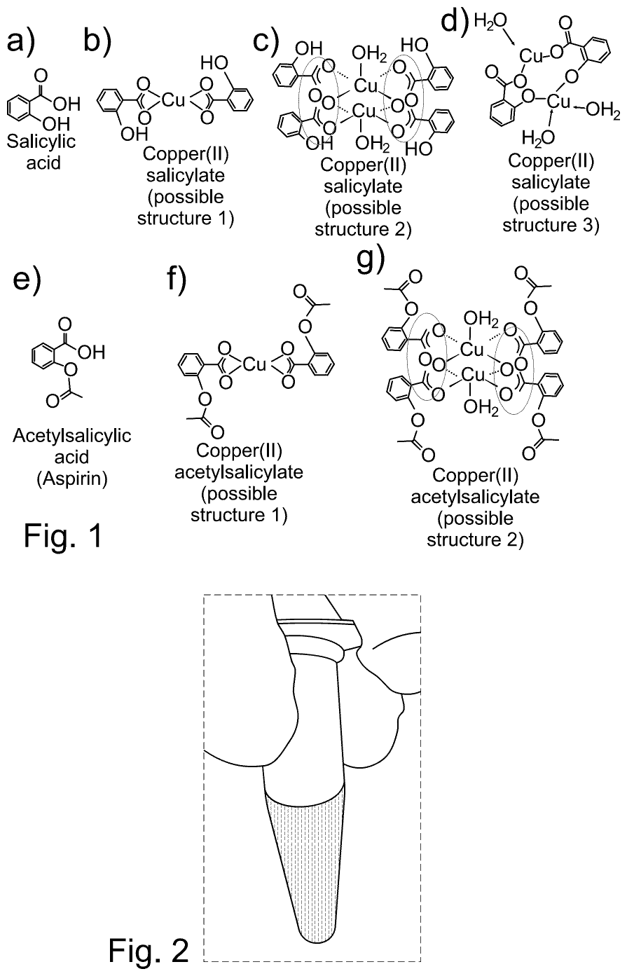

As will be well understood by those skilled in the art, a “copper complex” may actually exhibit multiple (sometimes inseparable) forms, and may therefore be provided as a mixture of said forms. As such, unless stated otherwise or otherwise incompatible in the given context, references to a (given) “copper complex” (whether in general or more specifically) suitably means said complex any mixture of alternative forms thereof. The complex may simple be defined by reference to the copper ion or atom and one or more specified ligands. For example, in the case of a copper-drug complex such as copper aspirinate (or copper aspirinate complex), said complex may be simply defined as a complex comprising copper (suitably copper(II)) and aspirin (or aspirinate), even though, as per FIGS. 1(f) and (g), said complex may be a mixture of multiple forms. It will be understood that other non-specified ligands, especially solvates such as water, may optionally complete any vacancies in co-ordination spheres. As such, the abovementioned example of a copper aspirinate complex may include multiple forms, including the binuclear hydrate form of FIG. 1(g).

### General Points and Advantages Regarding the Invention

The present invention provides lipid-based compositions of copper complexes that are capable of a variety of applications. Some of the compositions are themselves products which may have a direct use—e.g. topical compositions, such as creams, ointments, etc. Some of the compositions may be used to produce products—e.g. dispersions for oral administration, wound healing products (e.g. bandages, gauzes, etc. incorporated with the composition) or, again, topical compositions.

Compositions of the invention make a technical contribution over the prior art, and represent a particularly excellent formulation solution for copper complexes specifically. That the lipid-based compositions of the invention perform quite so well in the context of copper complexes specifically was considered surprising.

Compositions of the invention permit high loadings of copper complex, generally owing to the high levels of solubilisation and encapsulation achievable. That such high loadings of copper complexes are achievable was considered surprising, in the context of dry, hydrated, and aqueous formulations. It is especially surprising that such high loadings can be achieved without compromising metal complex bioavailability or stability. It is thought that the surprisingly high levels of solubilisation and/or encapsulation provided by the lipid-based systems described herein facilitates both bioavailability and stability (for both low and high loadings). The lipid-based system both protects the copper complexes, during storage and during administration (e.g. topically or orally/gastrointesintally), and provides an excellent vehicle for its delivery and ultimate release in a biological context.

The effective solubilisation and/or encapsulation of copper complexes within the lipid-based systems described herein is demonstrated by the minimal (or absence of) sedimentation (e.g. of the copper complex) observed following centrifugation of compositions of the invention. Such minimal (or lack of) sedimentation also testifies to the physical consistency of such compositions.

The advantages of the invention can be enjoyed in variety of use cases, particularly in medical applications (e.g. as vehicles for the topical or oral delivery of copper complexes, or in wound healing). Compositions of the invention may also be further processed (e.g. by mixing with other ingredients/components) into downstream products (e.g. oral dispersions) yet still benefit from the long-term storability afforded by substantially solid forms of compositions of the invention.

The lipid-based compositions of the invention are generally stable (especially physically stable) for long periods upon storage. Said compositions also allow copper complexes to remain chemically stable both during storage and during use (including upon the skin or within the body). Again it is thought that the lipid-based systems described herein are complementary to copper complexes to a surprising extent (apparently showing a high degree of structural and energetic complementarity in terms of molecular arrangements) owing to the excellent degree of solubilisation and/or encapsulation afforded to the copper complexes. The ‘cis’ conformation at the unsaturation is responsible for a kink in a lipid molecule causing its asymmetric bending. This, surprisingly, creates an explicit arrangement that complements the spatial orientation of copper complexes (especially copper-drug complexes, such as those defined herein); thus, facilitating copper complex localisation in this region. Such solubilisation and/or encapsulation appears to be a thermodynamic and a kinetic and phenomenon, respectively. Solubilisation preferably occurs in thermodynamically stable liquid crystalline systems, whereas encapsulation involves systematic contribution of hydrophobic forces, van Der Waals interactions and hydrogen-bonding along with the host lipid system's viscoelastic properties.

The lipid-based compositions of the invention perform surprisingly well even in the absence of high levels of surfactant(s). This is even so in aqueous dispersions of the compositions.

Lipid components employed in many formulations of the invention are common digestion products of consumed food fats (typically, triglycerides). Thus, lipid components herein are familiar to the internal milieu of the body which has a set mechanism for their breakdown and absorption in the bloodstream. This may facilitate a high degree of bioavailability of metal complexes.

### Composition

According to the invention, there is provided a composition. The composition may be as defined anywhere herein, including in paragraphs A1-A101, paragraphs B1-B427, or a combination thereof (since, as explained below, features of A1-A101 and B1-B427 may be combined by way of dependency or otherwise).

The composition suitably comprises a metal complex (or metal-ligand complex), which is suitably a metal-drug complex, most preferably a copper-drug complex, most suitably a copper(II)-drug complex. Suitable and preferred metal complexes for inclusion within compositions of the invention are defined herein.

The metal complex suitably comprises one or more metal atoms and/or ions (most preferably ions) and one or more ligands, wherein one or more of the one or more ligands is a drug (i.e. drug compound/molecule or drug ligand) or an ionised form of the drug. By way of example, the drug may be asprin (or aspirinate, which is an ionised form thereof), for instance copper(II) aspirinate.

As a person skilled in the art will readily understand, any metal-drug complex defined herein (e.g. copper aspirinate) may comprise other ligands (e.g. solvates or other appropriate ligands) in addition to one or more drug molecule(s).

The composition is suitably a lipid-based composition. The composition preferably comprises the metal complex (which is most preferably a copper complex, preferably a copper-drug complex, wherein the complex comprises drug ligands, such as aspirinate, as per copper aspirinate) and one or more lipids. In a particularly preferred embodiment, the composition comprises copper aspirinate and one or more lipids.

The one or more lipids are suitably one or more glycerides. The composition suitably comprises one or more glycerides, suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

The lipid (glyceride)-based systems of the invention are particularly advantageous for formulating metal complexes (especially copper-drug complexes, such as copper aspirinate) for a variety of applications. Such advantages are thought to stem from a surprising degree of complementarity between their respective chemical structures and physical properties, which synergistically afford molecular arrangements maximising solubilisation and/or encapsulation of the metal complex(es) within the lipid (glyceride)-based system. It is thought that crystalline lamellar structures and/or fluid lamellar structures, coupled with a special capacity for metal complexes (especially copper-drug complexes, such as copper aspirinate and copper salicylate) to bridge between individual lamella sheets and/or become encapsulated therein, may contribute to such advantages. Having unsaturated moieties, especially cis-alkene moieties, within relevant glyceride(s) is thought to be a key contributing factor in the particular case of the metal complexes described herein.

According to an aspect of the present invention, there is provided a composition comprising a metal-drug complex (suitably a copper-drug complex, such as copper aspirinate, copper salicylate, or relevant salt(s) and/or complex(es) thereof) and one or more glycerides, wherein the metal-drug complex comprises one or metal atoms and/or metal ions (suitably one or more copper atoms and/or copper ions, most preferably one or more copper(II) ions) co-ordinated with one or more drug molecules/ligands (or ionised forms of said drug molecule(s)/ligand(s)), suitably wherein at least one (preferably each) of glyceride(s) comprises at least one unsaturated moiety (preferably at least one alkene moiety, most preferably at least one cis-alkene moiety). Suitably the one or more glyceride(s) are one or more mono- and/or di-glycerides, though most preferably are one or more mono-glycerides.

### Type of Composition

In an embodiment, the composition is a topical composition (e.g. for use on a bodily surface, for example skin, eye(s), body cavity, and/or hair). Such a topical composition may be a composition as defined in any of paragraphs A1-A101, paragraphs B1-B427, or as defined anywhere else herein.

In an embodiment, the topical composition may be a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.

In a particular embodiment, the topical composition is a cream, an ointment, a foam, or a perfume (e.g. perfume, aftershave, cologne, or eau de toilette). In a particular embodiment, the topical composition is a cream. In another embodiment, the topical composition is an ointment. The present invention is particularly advantageous for topical compositions for the skin, permitting high concentrations of metal complexes to be rendered bioavailable for either local therapeutic effects or a more systemic therapeutic effect (e.g. following absorption through the skin). The invention also permits such topical compositions to exhibit a good “feel” on the skin.

In an alternative embodiment, the composition may be an oral topical composition, for example, a mouthwash, a rinse, an oral spray, a suspension, and/or a dental gel, suitably which is intended to be taken by mouth but are not intended to be ingested.

In an alternative embodiment, the composition is an ingestible composition (e.g. orally-administrable composition). Such an ingestible composition may be selected from a lozenge, a gel, a jelly, a pastille, a tablet, a capsule (e.g. a capsule containing the composition), a toffee, a nougat, a chewy candy, and/or a chewing gum. In a particular embodiment, the ingestible composition is (or is otherwise incorporated within) a pastille, a gel, or a jelly. In another embodiment, the ingestible composition comprises a gel component, gelling component, or gellable component. The gel component, gelling component, or gellable component is preferably animal- or plant-derived, most preferably plant-derived. In fact, most preferably, the composition or product into which said composition is incorporated is free of any animal-derived components/ingredients. The gel component, gelling component, or gellable component may comprise or consist of a polysaccharide or derivative thereof (e.g. x-carrageenan). The gel component, gelling component, or gellable component may comprise or consists of a gelatin, agar, and/or agarose. The ingestible composition may be (or may be otherwise incorporated within, for instance, by mixing the composition with a solvent such as water) a liquid, suitably a suspension, a dispersion, an emulsion, or a solution, most preferably a dispersion or emulsion. The ingestible composition may be a drink/beverage.

In an embodiment, the composition is (or is otherwise incorporated within) a pharmaceutical composition. Such a pharmaceutical composition is suitably for use as a medicament (or may otherwise be used in the manufacture of a medicament). In an embodiment, the product composition is for (or is for use in) bio/medicinal applications including antiulcer, anticancer, antimutagenic, antithrombotic and antifungal medications, microbial infections (e.g. composition as an antimicrobial composition), viral infections and fungal infections. It is also suitable to treat rheumatoid arthritis and similar disorders.

The composition may be in the form of (or may be otherwise incorporated within) an emulsion, a lotion, a milk, a liquid, a solid, a cream, a gel, a mousse, an ointment, a paste, a serum, a stick, a spray, a tonic, an aerosol, a foam, and/or a pencil. The composition may be an oil-in-water dispersion. The composition may be a water-in-oil dispersion.

In an embodiment, the composition is (or is otherwise incorporated within) a personal care composition. Said personal care composition may be selected from the group consisting of skin care compositions, skin creams, shaving compositions (e.g. shaving creams, gels, foams), moisturizers, lotions, body washes, body oils, and hair care compositions (e.g. shampoos and/or hair conditioners). The personal care composition may be a “leave-on-type” composition (e.g. moisturizing lotions, serums, creams) or a “rinse-off-type” composition (e.g. body washes, shampoos, hair conditioners, shower gels, skin cleansers, cleansing milks, shaving compositions). The personal care composition may be in the form of (or may be otherwise incorporated within) an implement (e.g. a powdered or unpowdered cosmetic applicator), a reusable or disposable wipe, a tissue, a towel, a diaper, a razor or other shaving device, a personal cleansing implement (e.g. a mesh shower sponge), a conventional sponge, a wash cloth, a swab, and/or a pen.

In an embodiment, the composition is a cosmetic composition (or may be a cosmetic product or may be incorporated within a cosmetic product—e.g. a cosmetic product comprising the composition). The cosmetic composition may be for controlling and/or reducing the formation of wrinkles and lines in the skin. The cosmetic composition may be for providing glowing skin.

In an embodiment, the composition is a wound care composition (or may be a wound care product or may be incorporated within a wound care product—e.g. a wound care product comprising the composition). The wound care (or wound healing) composition may be a wound care hydrogel composition, and therefor suitably comprises a hydrogel or hydrogel-forming component or compound—this may be a polysaccharide-based hydrogel or hydrogel-forming component or compound. The wound care composition may be incorporated within a wound care product—e.g. a wound care product comprising the composition. Such a wound care product may be selected from the group consisting of a wound dressing, a sutures, a staple, a gauze, a bandage, a plaster, a burn dressing, an artificial skin, a liposome or micelle formulation, a microcapsule, an aqueous vehicle for soaking gauze dressings. In an embodiment, the wound care composition (or product) may be for (or for use in) healing wounds, for example, healing cuts and scrapes, burns, ulcers, bed sores, fissures, haemorrhoids, and/or post-surgical wounds. The same properties that render compositions of the invention excellent for topical applications also apply to wound care compositions, and products derived therefrom.

The composition may be (or may be otherwise incorporated within) a textile—e.g. a textile (e.g. a clothing item) comprising the composition.

Metal Complex (Esp. Metal-Drug Complex)

The composition suitably comprises a metal complex (or metal-ligand complex). The metal complex suitably comprises one or more metal atoms and/or ions (most preferably ions) and one or more ligands. Preferably, one or more of the one or more ligands is a drug (i.e. drug compound or drug ligand). The metal complex is preferably a metal-drug complex (i.e. a or a plurality of metal(s) datively bonded to a drug molecule/compound, suitably via one or more, preferably two or more, lone pair(s) of electrons borne by the drug), and suitably the drug represents a multidentate ligand to the metal atom(s) and/or ion(s). The metal complex is suitably an aqueous-insoluble metal complex, most suitably an aqueous-insoluble metal-drug complex. For instance, in accordance with the solubility definitions of the US Pharmacopeia (USP), the metal complex is suitably practically insoluble (10,000 parts solvent required per part of solute), very slightly soluble (1,000-10,000 parts solvent required per part of solute), or slightly soluble (100-1,000 parts solvent required per part of solute), though preferably the metal complex is either practically insoluble or very slightly soluble, and is most preferably practically insoluble. The metal complex is suitably a transition metal complex. The transition metal complex is suitably an aqueous-insoluble transition metal complex, most suitably an aqueous-insoluble transition metal-drug complex.

The metal complex may be monometallic or polymetallic (e.g. bimetallic) in that the complex may comprise one or a plurality (e.g. two) metal atom(s) and/or ion(s) (most preferably ion(s)) within said complex. Where the metal complex is polymetallic, the polymetallic complex may be homometallic (i.e. where all of the plurality of metal atom(s) and/or ion(s) are derived from the same metal element) or heterometallic (i.e. where at least two of the metal atoms and/or ions are derived from different metal elements). Where the metal complex is polymetallic, the polymetallic complex may be homovalent (i.e. where all of the plurality of metal atom(s) and/or ion(s) are derived from the same or different metal elements but which all have the same valency or have the same numerical oxidation state) or heterovalent (i.e. where at least two of the metal atoms and/or ions, which may be derived from the same or different metal elements, have a different valency or oxidation state). Most preferably, the metal complex is monometallic, polymetallic (preferably bimetallic), or a mixture thereof, is homometallic, and most preferably is homovalent.

The metal complex is preferably a copper complex (or copper-ligand complex), most preferably a copper(II) complex (i.e. with copper in the 2+ oxidation state, or having a valency of 2). As such, the composition preferably comprises a copper complex. The copper complex suitably comprises one or more copper atoms and/or copper ions (most preferably copper ions, most preferably copper 2+ ions) and one or more ligands. Preferably, one or more of the one or more ligands is a drug (i.e. drug compound/molecule or drug ligand). The copper complex is suitably a copper-drug complex (i.e. a or a plurality of copper(s) datively bonded to a drug molecule/compound, suitably via one or more, preferably two or more, lone pair(s) of electrons borne by the drug), and suitably the drug represents a multidentate ligand to the copper atom(s) and/or copper ion(s) (most preferably copper(II) ions). The copper complex is suitably an aqueous-insoluble copper complex, most suitably an aqueous-insoluble copper-drug complex, most preferably an aqueous-insoluble copper(II)-drug complex.

The copper complex may be monometallic or polymetallic (e.g. bimetallic) in that the complex may comprise one or a plurality (e.g. two) copper atom(s) and/or ion(s) (most preferably ion(s)) within said complex. Where the copper complex is polymetallic, the polymetallic complex may be homometallic (i.e. where all of the plurality of metal atom(s) and/or ion(s) are derived from the element copper) or heterometallic (i.e. where at least one of the metal atoms and/or ions are derived from the element copper, and at least one other of the metal atoms and/or ions are derived from different metal elements). Where the copper complex is polymetallic, the polymetallic complex may be homovalent (i.e. where all of the plurality of metal atom(s) and/or ion(s), whether or not all or some are derived from the element copper, which all have the same valency or have the same numerical oxidation state) or heterovalent (i.e. where at least two of the metal atoms and/or ions, whether or not all or some are derived from the element copper, have a different valency or oxidation state). Most preferably, the copper complex is monometallic, polymetallic (preferably bimetallic), or a mixture thereof, is homometallic (i.e. all metal atoms and/or ions are derived from copper), and most preferably is homovalent (preferably all copper atoms and/or ions have the same valency or oxidation state).

The composition preferably comprises a metal complex (suitably as defined herein), which is most preferably a copper complex (suitably as defined herein), wherein the metal complex comprises one or more metal atoms and/or ions (most preferably ions) and one or more ligands, wherein one or more of the one or more ligands is a drug (i.e. drug compound/molecule or drug ligand) or ionised form of said drug. Preferably, the drug is a drug compound/molecule or an ionised form thereof (e.g. carboxylate corresponding to a carboxylic acid moiety), which is datively bonded (suitably in a monodentate or multidentate fashion, suitable via one or more lone pair(s) of electrons borne by the drug) to one or more of the metal atoms and/or metal ions of the metal complex. By way of example, the drug may be aspirin (or aspirinate). By way of an alternative example, the drug may be salicylic acid (salicylate) (or a derivative thereof).

As a person skilled in the art will readily understand, any metal-drug complex defined herein (e.g. copper aspirinate) may comprise other ligands (e.g. solvates or other appropriate ligands) in addition to one or more drug molecule(s).

Most preferably, the metal complex is a metal-drug complex comprising one or more copper ions (preferably copper(II) ions) and one or more drug molecule(s) (or ionised form(s) thereof) as defined herein.

In a particular embodiment, the metal complex is copper(II) salicylate (or any suitable complex or salt thereof).

In a particular, and most preferred, embodiment the metal complex is copper(II) aspirinate (or any suitable complex or salt thereof).

Suitably, the composition comprises 0.0001-50 wt % metal complex. Suitably, the composition comprises 0.001-40 wt % metal complex. Suitably, the composition comprises 0.005-30 wt % metal complex.

Suitably, the composition (especially where the composition has no or low water content, and little to no encapsulation of the metal complex) comprises 0.0001-1 wt % metal complex. Suitably, the composition comprises 0.001-0.1 wt % metal complex. Suitably, the composition comprises 0.005-0.05 wt % metal complex. Suitably, the composition comprises about 0.01 wt % metal complex.

Suitably, the composition (especially where the composition has no or low water content, and at least some encapsulation of the metal complex) comprises 1-30 wt % metal complex. Suitably, the composition comprises 2-20 wt % metal complex. Suitably, the composition comprises 5-15 wt % metal complex. Suitably, the composition comprises 10-12 wt % metal complex.

Suitably, the composition (especially where the composition has at least some water, but little to no encapsulation of the metal complex) comprises 0.01-20 wt % metal complex. Suitably, the composition comprises 0.1-15 wt % metal complex. Suitably, the composition comprises 1-10 wt % metal complex. Suitably, the composition comprises about 2-7 wt % metal complex. Suitably, the composition comprises about 1-3 wt % (suitably 2-2.5 wt %) metal complex. Suitably, the composition comprises about 4-8 wt % (suitably 5-7 wt %) metal complex.

Suitably, the composition (especially where the composition has at least some water, and at least some encapsulation of the metal complex) comprises 1-50 wt % metal complex. Suitably, the composition comprises 5-40 wt % metal complex. Suitably, the composition comprises 10-30 wt % metal complex. Suitably, the composition comprises 20-30 wt % metal complex. Suitably, the composition comprises 23-26 wt % metal complex.

Suitably, the composition (especially where the composition is aqueous) comprises 0.001-5 wt % metal complex. Suitably, the composition comprises 0.01-1 wt % metal complex. Suitably, the composition comprises 0.02-0.5 wt % metal complex. Suitably, the composition comprises 0.02-0.06 wt % metal complex. Suitably, the composition comprises 0.1-0.4 wt % metal complex.

It will be understood by those skilled in the art that embodiments of the invention defined herein as comprising a metal complex (be it defined generically, specifically, or at any intermediate level of generalisation therebetween) may be defined as comprising one or more of said metal complexes, suitably each of which is independently as defined herein in relation to a metal complex. In such cases, amounts, concentrations, and ratios, defined herein in relation to a metal complex may be combined amounts, concentrations, and ratios, of all of the one or more metal complexes (especially applicable where there are two or more metal complexes).

References herein to a “metal complex” may, in alternative embodiments, be replaced by the term “metal compound”. Such a metal compound preferably has one or more metal ion(s) (or atom(s)). Such a metal compound may be free of a drug compound or drug ligand. For instance, the metal compound may be a metal oxide. Preferably such a metal compound is an antimicrobial and/or antifungal metal compound. Such a metal compound is suitably a copper compound. The compound may be an inorganic copper compound. The compound may be a copper(I) compound. The compound may be a copper (II) compound. However, the copper compound may be a copper complex, such as a copper-drug complex (or salt thereof) as defined herein.

It will be understood by those skilled in the art that embodiments of the invention defined herein as comprising a metal compound (be it defined generically, specifically, or at any intermediate level of generalisation therebetween) may be defined as comprising one or more of said metal compounds, suitably each of which is independently as defined herein in relation to a metal compound. In such cases, amounts, concentrations, and ratios, defined herein in relation to a metal compound may be combined amounts, concentrations, and ratios, of all of the one or more metal compounds (especially applicable where there are two or more metal compounds).

### Metal Ion(s) (or Atom(s))

The metal complex, as defined herein, suitably comprises one or more metal atoms and/or ions (most preferably ions), suitably along with one or more ligands (suitably as defined herein). More suitably, the metal complex comprises one or more metal ions. More suitably, the metal complex comprises one or more transition-metal ions. Most suitably, the metal complex comprises one or more copper ions. Preferably, the metal complex comprises one or more copper(II) ions (i.e. Cu²⁺ ions).

The metal complex is suitably monometallic or polymetallic (e.g. bimetallic). Suitably, where the metal complex is polymetallic (especially when bimetallic), the polymetallic complex is homometallic, in that all of the plurality of ions are derived from the same metal element (preferably copper), and homovalent. in that all of the plurality of metal ions (preferably copper ions) have the same valency (e.g. Cu²⁺). The metal complex may be a mixture of monometallic and polymetallic (preferably bimetallic) forms of said metal complex.

The metal ion(s) (or, where relevant, metal atom(s)) is suitably co-ordinated to one or more drug molecule(s) and optionally to one or more additional ligands.

### Drug Molecule (Ligand of Metal-Drug Complex)

The composition suitably comprises a drug molecule (which includes any ionised forms thereof, e.g. carboxylate if the drug molecular contains a carboxylic acid moiety) which is most-suitably a ligand within a metal-drug complex (suitably copper-drug complex). The metal-drug complex suitably comprises one or more (a plurality of) drug molecule ligands. As such, the drug molecule is suitably any drug molecule capable of co-ordinating to (or with) the metal atom(s) and/or metal ion(s) within the metal-drug complex. The drug molecule (which includes any ionised form thereof) may be abbreviated simply to “drug”.

The drug molecule suitably comprises one or more moieties capable of donating one or more lone pair(s) of electrons. The drug molecule is suitably datively bonded to one or more metal atoms and/or metal ions of the metal-drug complex. The drug molecule suitably co-ordinates within the complex in a multidentate fashion, be it with a single metal atom or metal ion or with two or more metal atoms or metal ions.

The drug molecule is suitably a carboxylic acid or (given that the definition of a drug molecule includes any ionised forms thereof) a carboxylate. Suitably the carboxylic acid (or carboxylate) co-ordinates in a bidentate fashion within the complex, be it with a single metal atom or metal ion or with two or more metal atoms or metal ions.

In an embodiment, the drug molecule is defined by Formula I:

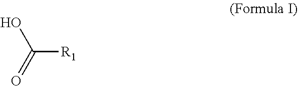

wherein R₁ is an optionally substituted organic (i.e. carbon-containing) group suitably which together with the carboxylic acid moiety completes the drug molecule;
or any ionised form thereof.

R₁ is suitably an optionally substituted hydrocarbyl group (suitably optionally substituted by one or more moieties independently selected from Rₓ), wherein adjacent carbon atoms within the hydrocarbyl group are optionally separated by a linker -L-, for instance by the insertion thereinto of a heteroatom (e.g. 0, S) or heteroatom-containing moiety (e.g. 0, S, SO, SO₂, N(Rₐ), CO, C(O)O, OC(O), OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group).

R₁ suitably is or comprises one or more optionally substituted ring systems (suitably optionally substituted by one or more moieties independently selected from Rₓ), fused or otherwise, wherein adjacent carbon atoms within the hydrocarbyl group are optionally separated by a linker -L-, for instance by the insertion thereinto of a heteroatom (e.g. 0, S) or heteroatom-containing moiety (e.g. O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group).

R₁ suitably is or comprises an optionally substituted moiety (suitably optionally substituted by one or more moieties independently selected from Rₓ) selected from the group consisting of aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl, heterocyclyl-(1-6C)alkyl, and any combination thereof.

R₁ suitably is or comprises an optionally substituted moiety (suitably optionally substituted by one or more moieties independently selected from Rₓ) selected from of aryl, aryl-(1-6C)alkyl, heteroaryl, heteroaryl-(1-6C)alkyl, and any combination thereof.

R₁ suitably is or comprises an optionally substituted (suitably optionally substituted by one or more moieties independently selected from Rₓ) aryl.

R₁ suitably is or comprises an optionally substituted (suitably optionally substituted by one or more moieties independently selected from Rₓ) phenyl.

Rₓ is suitably selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

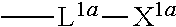

wherein:
L^{1a} is absent or is selected from O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any Rₓ group is optionally further substituted with one or more Rₓ groups as defined above.

Rₓ is suitably selected from hydrogen, halogeno, trifluoromethyl, cyano, isocyano, nitro, hydroxy, mercapto, amino, formyl, carboxy, carbamoyl, ureido, (1-8C)alkyl, (1-8C)hydroxyalkyl, (1-6C)alkoxy, (1-6C)alkylamino, (1-6C)dialkylamino, (2-6C)alkanoyl, (2-6C)alkanoyloxy, or from a group of the formula:

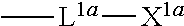

wherein:
L^{1a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein where Rₐ and R_{b}, which may be the same or different, are each independently selected from hydrogen or (1-8C)alkyl; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl; wherein any Rₓ group is optionally further substituted with one or more Rₓ groups as defined above.

Rₓ is suitably selected from halogeno, trifluoromethyl, cyano, hydroxy, amino, carboxy, (1-8C)alkyl, (1-8C)hydroxyalkyl, (1-6C)alkoxy, (2-6C)alkanoyl, (2-6C)alkanoyloxy.

Rₓ is suitably selected from hydroxy, (1-6C)alkoxy, and (2-6C)alkanoyloxy.

Rₓ is suitably selected from hydroxy and (2-6C)alkanoyloxy.

Rₓ is suitably selected from hydroxy (—OH) and acetoxy (—OAc or —OC(O)CH₃).

The linker, -L-, is suitably selected from O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group.

In an embodiment, the drug molecule is defined by Formula II:

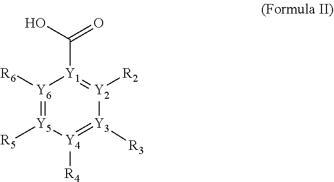

wherein Y₁ is C, and each of Y₂, Y₃, Y₄, Y₅, and Y₆ are independently selected from C, N, and O (where each of R₂, R₃, R₄, R₅, and R₆ are correspondingly compatible, and may suitably be absent where their corresponding Y atom is 0 or N);
wherein each of R₂, R₃, R₄, R₅, and R₆ are independently either absent (e.g. if the corresponding Y₁-Y₆ to which they are connected is either 0 or N), hydrogen, or an Rₓ (suitably as defined herein);
or any ionised form thereof.

In an embodiment, the drug molecule is defined by Formula III:

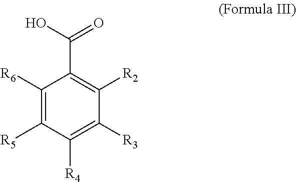

wherein each of R₂, R₃, R₄, R₅, and R₆ are independently either hydrogen or an Rₓ (suitably as defined herein, though more suitably where Rₓ is selected from hydroxy, (1-6C)alkoxy, and (2-6C)alkanoyloxy, and most suitably wherein Rₓ is selected from hydroxy and acetoxy);
or any ionised form thereof.

In an embodiment, the drug molecule is defined by Formula IV:

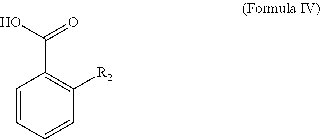

wherein R₂ is either hydrogen or an Rₓ (suitably as defined herein, though more suitably where Rₓ is selected from hydroxy, (1-6C)alkoxy, and (2-6C)alkanoyloxy, and most suitably wherein Rₓ is selected from hydroxy and acetoxy);
or any ionised form thereof.

In an embodiment, the drug molecule is defined by Formula V:

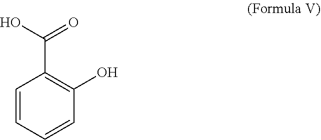

or any ionised form thereof.

In an embodiment, the drug molecule is defined by Formula VI:

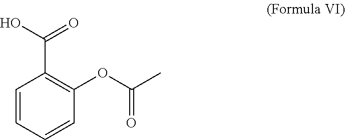

or any ionised form thereof.

In an embodiment, the drug molecule is salicylic acid.

In an embodiment, and most preferably, the drug molecule is aspirin (acetylsalicylic acid).

### Glycerides

The composition suitably comprises one or more glycerides, suitably in addition to a metal complex (suitably as defined herein). As such, the composition suitably comprises a glyceride. Unless otherwise qualified (e.g. as two or more), the term “glyceride(s)” refers to one or more glycerides.

The composition suitably comprises a mono- and/or di-glyceride. Most preferably, the composition comprises a monoglyceride.

Suitably, the composition comprises two or more glycerides. As such, suitably the composition comprises a first glyceride and a second glyceride. The composition suitably comprises a first glyceride and a second glyceride in a weight ratio between 1:99 and 99:1. The composition suitably comprises a first glyceride and a second glyceride in a weight ratio between 1:99 and 90:10. The composition suitably comprises a first glyceride and a second glyceride in a weight ratio between 10:90 and 50:50. The composition suitably comprises a first glyceride and a second glyceride in a weight ratio between 20:80 and 40:60. The composition suitably comprises a first glyceride and a second glyceride in a weight ratio between 25:75 and 35:65. The composition suitably comprises a first glyceride and a second glyceride in a weight ratio between 27:73 and 30:70.

Preferably, the aforesaid glyceride(s) is/are mono- and/or di-glyceride(s), though most preferably the aforesaid glyceride(s) is/are mono-glyceride(s). Mono-glyceride(s) are thought to facilitate particularly complementary molecular arrangements for solubilising and/or encapsulating metal complexes (suitably as defined herein), and the inventors have shown that higher glycerides tend to reduce such solubilisation/encapsulation capacities.

Suitably, at least one of the glyceride(s) has a melting point of 25-55° C. Suitably, at least two (preferably the two which are mentioned/defined) of the glyceride(s) have a melting point of 25-55° C. Suitably, each defined glyceride(s) each has a melting point of 25-55° C. Suitably, at least one of the glyceride(s) has a melting point of 30-45° C. Suitably at least two (preferably the two which are mentioned/defined) of the glyceride(s) have a melting point of 30-45° C. Suitably, each defined glyceride(s) each has a melting point of 30-45° C. Suitably, at least one of the glyceride(s) has a melting point of 34-41° C. Suitably, at least two (preferably the two which are mentioned/defined) of the glyceride(s) have a melting point of 34-41° C. Suitably, each defined glyceride(s) each has a melting point of 34-41° C.

Suitably, the, each, or at least one of the, glyceride(s) comprises one or more unsaturated moieties. Suitably, the, each, or at least one of the, glyceride(s) comprises between one and two unsaturated moieties. Suitably, the, each, or at least one of the, glyceride(s) comprises one (i.e. a single, one and only one) unsaturated moiety. Suitably, the, each, or at least one of the, glyceride(s) comprises two (i.e. two and only two) unsaturated moieties.

Suitably, the composition comprises two glycerides, a first glyceride with one (i.e. one and only one) unsaturated moiety and a second glyceride with two (i.e. two and only two) unsaturated moieties. Suitably the respective first and second glyceride may be present in one of the aforementioned weight ratios.

Suitably, the, each, or at least one of the, unsaturated moiety(ies) is/are an alkene moiety(ies).

Suitably, the, each, or at least one of the, alkene moiety(ies) is/are cis-alkene moiety(ies).

Suitably, the, each, or at least one of the, glyceride(s) comprises one, two, or three fatty acid(s) (suitably respectively comprises one or two fatty acids in the case of mono- and/or diglyceride, and suitably respectively comprises only one fatty acid in the case of mono-glycerides) condensed with glycerol.

Suitably, the, each, or at least one of the, fatty acid(s) is/are aliphatic fatty acids. Suitably, the, each, or at least one of the, fatty acid(s) is/are linear (i.e. unbranched) fatty acids.

Suitably, the, each, or at least one of the, fatty acid(s) is/are C₆-C₃₀ fatty acid(s) (i.e. having a carbon chain that is 6-30 carbons in length, including the carboxylate carbon). Suitably, the, each, or at least one of the, fatty acid(s) is/are C₈-C₂₀ fatty acid(s). Suitably, the, each, or at least one of the, fatty acid(s) is/are C₁₈ fatty acid(s).

Suitably, the, each, or at least one of the, fatty acid(s) is/are aliphatic C₆-C₃₀ fatty acid(s) (i.e. having a carbon chain that is 6-30 carbons in length, including the carboxylate carbon). Suitably, the, each, or at least one of the, fatty acid(s) is/are aliphatic C₈-C₂₀ fatty acid(s). Suitably, the, each, or at least one of the, fatty acid(s) is/are aliphatic C₁₈ fatty acid(s).

Suitably, the, each, or at least one of the, fatty acid(s) is/are linear C₆-C₃₀ fatty acid(s) (i.e. having a carbon chain that is 6-30 carbons in length, including the carboxylate carbon). Suitably, the, each, or at least one of the, fatty acid(s) is/are linear C₈-C₂₀ fatty acid(s). Suitably, the, each, or at least one of the, fatty acid(s) is/are linear C₁₈ fatty acid(s).

Suitably, the, each, or at least one of the, fatty acid(s) comprises one or more unsaturated moieties. Suitably, the, each, or at least one of the, fatty acid(s) comprises between one and two unsaturated moieties. Suitably, the, each, or at least one of the, fatty acid(s) comprises one (i.e. a single, one and only one) unsaturated moiety. Suitably, the, each, or at least one of the, fatty acid(s) comprises two (i.e. two and only two) unsaturated moieties.

Suitably, the composition comprises a first glyceride comprising a fatty acid (suitably only a single fatty acid, as per a mono-glyceride) with one or more unsaturated moiety(ies), and a second glyceride comprising a fatty acid (suitably only a single fatty acid, as per a mono-glyceride) with one or more unsaturated moiety(ies).

Suitably, the composition comprises a first glyceride comprising a fatty acid (suitably only a single fatty acid, as per a mono-glyceride) with one (i.e. one and only one) unsaturated moiety, and a second glyceride comprising a fatty acid (suitably only a single fatty acid, as per a mono-glyceride) with two (i.e. two and only two) unsaturated moieties.

Suitably, the, each, or at least one of the, unsaturated moiety(ies) is/are an alkene moiety(ies). Suitably, the, each, or at least one of the, alkene moiety(ies) is/are cis-alkene moiety(ies).

Suitably, wherein the, each, or at least one of the, fatty acid(s) is oleic acid. Suitably, wherein the, each, or at least one of the, fatty acid(s) is linoleic acid.

Preferably, the composition comprises glyceryl monooleate (monoolein).

Suitably, the composition comprises glyceryl monolinoleate (monolinolein).

Preferably, the composition comprises glyceryl monooleate (monoolein) and glyceryl monolinoleate (monolinolein). Suitably, the composition comprises glyceryl monooleate and glyceryl monolinoleate in a weight ratio between 1:99 and 90:10. Suitably, the composition comprises glyceryl monooleate and glyceryl monolinoleate in a weight ratio between 10:90 and 50:50. Suitably, the composition comprises glyceryl monooleate and glyceryl monolinoleate in a weight ratio between 20:80 and 40:60. Suitably, the composition comprises glyceryl monooleate and glyceryl monolinoleate in a weight ratio between 25:75 and 35:65. Suitably, the composition comprises glyceryl monooleate and glyceryl monolinoleate in a weight ratio between 27:73 and 30:70.

The use in lipid-based systems of the invention of a second glyceride in addition to a first, especially where the first is glyceryl monooleate and the second is glyceryl monolinoleate, can significantly increase solubilisation and/or encapsulation capacity for metal complexes (suitably as defined herein). In fact, a second glyceride (especially where it, like the first, contains one or more unsaturated moieties), such as glyceryl monolinoleate, can unlock previously unobtainable encapsulation potential to permit lipid-based systems to hold onto metal complexes (suitably as defined herein) way beyond their natural solubility limit, often even without risk of sedimentation upon storage and/or centrifugation (sedimentation is otherwise quite common for over-saturated lipid-based systems).

Preferably, the metal complex (suitably as defined herein) is solubilised and/or encapsulated within the glyceride(s) (or relevant sub-definition(s) thereof). Preferably, the metal complex (suitably as defined herein) is dissolved within the glyceride(s). Suitably, the metal complex (suitably as defined herein) is encapsulated within the glyceride(s). Suitably, the metal complex (suitably as defined herein) is both dissolved within and encapsulated within the glyceride(s).

Suitably, the composition comprises lamella structures comprising both the metal complex (suitably as defined herein) and the glyceride(s). Lamella structures are especially predominant in dehydrated (or low water content) systems.

Suitably, the lamella structures are characterised by a liquid crystalline fluid lamellar (L,) phase comprising both the metal complex and the glyceride(s).

Suitably, the metal complex (or relevant sub-definition(s) thereof) and the glyceride(s) (or relevant sub-definition(s) thereof) are present within the composition at a defined relative weight ratio. Such a ratio may apply regardless of the level of dilution of the composition as a whole.

Suitably, the weight ratio of the metal complex to the glyceride(s) is between 0.01:1000 and 900:1000. Suitably, the weight ratio of the metal complex to the glyceride(s) is between 0.1:1000 and 700:1000. Suitably, the weight ratio of the metal complex to the glyceride(s) is between 0.5:1000 and 600:1000.

Suitably (especially where the composition has no or low water content, and little to no encapsulation of the metal complex), the weight ratio of the metal complex to the glyceride(s) is between 0.0001:1000 and 50:1000. Suitably, the weight ratio of the metal complex to the glyceride(s) is between 0.001:1000 and 10:1000. Suitably, the weight ratio of the metal complex to the glyceride(s) is between 0.1:1000 and 5:1000. Suitably, the weight ratio of the metal complex to the glyceride(s) is between 0.5:1000 and 1.5:1000.

Suitably (especially where the composition has no or low water content, and at least some encapsulation of the metal complex), the weight ratio of the metal complex to the glyceride(s) is between 0.1:1000 and 500:1000. Suitably, the weight ratio of the metal complex to the glyceride(s) is between 10:1000 and 400:1000. Suitably, the weight ratio of the metal complex to the glyceride(s) is between 50:1000 and 200:1000. Suitably, the weight ratio of the metal complex to the glyceride(s) is between 90:1000 and 160:1000.

Suitably (especially where the composition has at least some water, but little to no encapsulation of the metal complex), the weight ratio of the metal complex to the glyceride(s) is between 0.1:1000 and 300:1000. Suitably, the weight ratio of the metal complex to the glyceride(s) is between 1:1000 and 200:1000. Suitably, the weight ratio of the metal complex to the glyceride(s) is between 10:1000 and 150:1000. Suitably, the weight ratio of the metal complex to the glyceride(s) is between 20:1000 and 130:1000. Suitably, the weight ratio of the metal complex to the glyceride(s) is between 10:1000 and 70:1000 (suitably between 20:1000 and 50:1000). Suitably, the weight ratio of the metal complex to the glyceride(s) is between 50:1000 and 150:1000 (suitably between 90:1000 and 120:1000).

Suitably (especially where the composition has at least some water, and at least some encapsulation of the metal complex), the weight ratio of the metal complex to the glyceride(s) is between 10:1000 and 900:1000. Suitably, the weight ratio of the metal complex to the glyceride(s) is between 50:1000 and 700:1000. Suitably, the weight ratio of the metal complex to the glyceride(s) is between 100:1000 and 600:1000. Suitably, the weight ratio of the metal complex to the glyceride(s) is between 200:1000 and 580:1000. Suitably, the weight ratio of the metal complex to the glyceride(s) is between 500:1000 and 550:1000.

Suitably (especially where the composition is aqueous), the weight ratio of the metal complex to the glyceride(s) is between 0.1:1000 and 600:1000. Suitably, the weight ratio of the metal complex to the glyceride(s) is between 1:1000 and 200:1000. Suitably, the weight ratio of the metal complex to the glyceride(s) is between 5:1000 and 100:1000. Suitably, the weight ratio of the metal complex to the glyceride(s) is between 2:1000 and 20:1000. Suitably, the weight ratio of the metal complex to the glyceride(s) is between 30:1000 and 70:1000.

Suitably, the composition comprises 0.1-99.999 wt % glyceride(s) (i.e. 0.1-99.999 wt % of one or more glycerides). Suitably, the composition comprises 1-99.99 wt % glyceride(s). Suitably, the composition comprises 4-99.99 wt % glyceride(s).

Suitably, the composition (especially where the composition has no or low water content, and little to no encapsulation of the metal complex) comprises 10-99.9999 wt % glyceride(s). Suitably, the composition comprises 50-99.999 wt % glyceride(s). Suitably, the composition comprises 80-99.995 wt % glyceride(s). Suitably, the composition comprises about 99.99 wt % glyceride(s).

Suitably, the composition (especially where the composition has no or low water content, and at least some encapsulation of the metal complex) comprises 10-99 wt % glyceride(s). Suitably, the composition comprises 50-95 wt % glyceride(s). Suitably, the composition comprises 80-93 wt % glyceride(s). Suitably, the composition comprises 88-90 wt % glyceride(s).

Suitably, the composition (especially where the composition has at least some water, but little to no encapsulation of the metal complex) comprises 5-95 wt % glyceride(s). Suitably, the composition comprises 30-90 wt % glyceride(s). Suitably, the composition comprises 40-80 wt % glyceride(s). Suitably, the composition comprises about 50-70 wt % glyceride(s). Suitably, the composition comprises about 55-60 wt % glyceride(s).

Suitably, the composition (especially where the composition has at least some water, and at least some encapsulation of the metal complex) comprises 5-95 wt % glyceride(s). Suitably, the composition comprises 20-80 wt % glyceride(s). Suitably, the composition comprises 30-70 wt % glyceride(s). Suitably, the composition comprises 40-50 wt % glyceride(s). Suitably, the composition comprises 43-47 wt % glyceride(s).

Suitably, the composition (especially where the composition is aqueous) comprises 0.01-30 wt % glyceride(s). Suitably, the composition comprises 0.1-20 wt % glyceride(s). Suitably, the composition comprises 1-10 wt % glyceride(s). Suitably, the composition comprises 2-8 wt % glyceride(s). Suitably, the composition comprises 4-6 wt % glyceride(s).

Suitably, the stipulated (i.e. the defined) glyceride(s) (or relevant sub-definition(s) thereof) are the predominant glyceride(s), and suitably the predominant lipid(s) (e.g. fatty-acid-containing compounds), within the composition. Suitably, all glyceride(s) (including predominant glyceride(s)) and lipids within the composition are collectively termed lipid compounds.

Suitably, the stipulated (i.e. the defined) glyceride(s) (or relevant sub-definition(s) thereof) constitute at least 50 wt % of the total amount of glyceride(s) present within the composition (i.e. the “50 wt %” is relative to total glyceride(s) rather than the composition as a whole), and preferably constitute at least 50 wt % of the total amount of lipid(s) present within the composition (i.e. the “50 wt %” is relative to total lipid(s) rather than the composition as a whole). All glyceride(s) (including predominant glyceride(s)) and other lipids within the composition are collectively termed lipid compounds or just “lipids”. Suitably, the stipulated (or defined) glyceride(s) constitute at least 70 wt % of the total amount of glyceride(s) present within the composition. Suitably, the stipulated (or defined) glyceride(s) constitute at least 85 wt % of the total amount of glyceride(s) present within the composition. Suitably, the stipulated (or defined) glyceride(s) constitute at least 89 wt % of the total amount of glyceride(s) present within the composition. Suitably, the stipulated (or defined) glyceride(s) constitute at least 95 wt % of the total amount of glyceride(s) present within the composition.

Suitably, the stipulated (or defined) glyceride(s) constitute at least 70 wt % of the total amount of lipids present within the composition. Suitably, the stipulated (or defined) glyceride(s) constitute at least 85 wt % of the total amount of lipids present within the composition. Suitably, the stipulated (or defined) glyceride(s) constitute at least 89 wt % of the total amount of lipids present within the composition. Suitably, the stipulated (or defined) glyceride(s) constitute at least 95 wt % of the total amount of lipids present within the composition.

Suitably, glyceride(s) (stipulated and/or unstipulated) constitute at least 70 wt % of the total amount of lipids present within the composition. Suitably, glyceride(s) (stipulated and/or unstipulated) constitute at least 85 wt % of the total amount of lipids present within the composition. Suitably, glyceride(s) (stipulated and/or unstipulated) constitute at least 95 wt % of the total amount of lipids present within the composition. Suitably, glyceride(s) (stipulated and/or unstipulated) constitute at least 99 wt % of the total amount of lipids present within the composition.

### Solvent

The composition may comprise a solvent (e.g. water). The solvent is preferably a polar solvent, more preferably a protic solvent, most preferably water. Preferably, the only solvent present within the composition is water (or else preferably solvents other than water constitute at most 1 wt % of the composition, preferably at most 0.1 wt %, most preferably at most 0.01 wt %). A certain quantity of water can be particularly advantageous for increasing the metal complex loading capacity within the lipid-based systems of the invention—such water can not only increase solubilisation but can, surprisingly, also increase encapsulation capacity.

Suitably, the composition comprises 0-99 wt % water. Suitably, the composition comprises 0.0001-98 wt % water.

Suitably, the composition comprises 0.001-95 wt % water.

Suitably, the composition comprises less than or equal to 80 wt % water (i.e. the composition has at most 80 wt % water, and may contain 0 wt % water). Suitably, the composition comprises less than or equal to 60 wt % water (i.e. the composition has at most 60 wt % water, and may contain 0 wt % water). Suitably, the composition comprises less than or equal to 40 wt % water (i.e. the composition has at most 40 wt % water, and may contain 0 wt % water).

Suitably, the composition (especially where the composition has no or low water content, and either little to no encapsulation or at least some encapsulation of the metal complex) comprises less than or equal to 5 wt % water (i.e. the composition has at most 5 wt % water, and may contain 0 wt % water). Suitably, the composition comprises less than or equal to 2 wt % water (and may contain 0 wt % water). Suitably, the composition comprises less than or equal to 1 wt % water (and may contain 0 wt % water). These are relatively unhydrated/dehydrated compositions.

Suitably, the composition (especially where the composition has at least some water, but little to no encapsulation of the metal complex) comprises 1-60 wt % water, preferably 10-60 wt % water, (especially where the composition has at least some water, but little to no encapsulation of the metal complex—i.e. a hydrated composition with a moderate amount of water). Such compositions are especially advantageous for solubilising and encapsulating large amounts of metal complex. Suitably, the composition comprises 15-60 wt % water. Suitably, the composition comprises 25-50 wt % water. Suitably, the composition comprises 30-45 wt % water. Suitably, the composition comprises 35-40 wt % water. Suitably, these compositions (with a moderate amount of water) are formed by adding a required amount of water to an unhydrated/dehydrated composition (suitably with no more than 5 wt % water, such as a composition of B369-B372), and suitably thereafter mixing, suitably with sufficient heating to melt the unhydrated/dehydrated composition or at least the glyceride(s) thereof. Wherever a metal complex is to be added (e.g. to form a hydrated composition containing significant quantities of metal complex), suitably metal complex addition is performed by mixing additional metal complex with the unhydrated/dehydrated composition (preferably heated so as to be in a melted state) before the addition of water. Products are suitably stored at or below 20° C.

Suitably, the composition (especially where the composition has at least some water, and at least some encapsulation of the metal complex) comprises 1-60 wt % water, preferably 10-60 wt % water. Suitably, the composition comprises 10-50 wt % water. Suitably, the composition comprises 20-40 wt % water. Suitably, the composition comprises 25-35 wt % water. Suitably, the composition comprises 28-32 wt % water.

Suitably, the composition (especially where the composition is aqueous) comprises 85-99.9 wt % water (i.e. very hydrated, suitably a dispersion or emulsion). Suitably, the composition comprises 85-99 wt % water. Such compositions may suitable include a surfactant. Suitably, the composition comprises 70-98 wt % water. Suitably, the composition comprises 90-97 wt % water. Suitably, the composition comprises 93-96 wt % water. Such very hydrated compositions may suitable include a surfactant. Such a very hydrated composition is suitably formed by adding a required amount of water to an unhydrated/dehydrated composition or a moderately hydrated composition (e.g. of B369-B372), and suitably thereafter mixing, suitably with sufficient heating to melt the unhydrated/dehydrated or moderately hydrated composition or at least the glyceride(s) thereof.

Additional Ingredient(s) and Limitations in relation thereto

The composition may comprise one or more additional ingredients, or else the composition may be otherwise characterised by limits in relation to (e.g. a maximum amount/concentration/ratio of) said one or more additional ingredients—as such the composition may comprise said additional ingredient(s), may be free of said additional ingredient(s), or may have a stipulated maximum quantity thereof.

The one or more additional ingredients may be ingredients selected from the group consisting of additional active(s), additional lipid(s) (e.g. fatty acid(s)), natural oil(s), bile salt(s) (or bile acid(s)), hydrophilic molecule(s), hydrophobic molecule(s), amphiphilic molecule(s), surfactant(s), or any combination thereof.

Suitably, save for additional active(s) or the metal complex, the composition consists of only GRAS grade components. Suitably the entire composition contains only GRAS grade components.

### Additional Lipids (or Glycerides)

The one or more additional ingredients may comprise one or more additional lipids.

The one or more additional lipids may comprise one or more glyceride(s) other than those stipulated (e.g. those stipulated above or in paragraphs B141-B198 below), suitably where said one or more glyceride(s) other than those stipulated may be referred to as “unstipulated” glyceride(s). Suitably, the weight ratio of unstipulated glyceride(s) (e.g. those other than the glyceride(s) stipulated in B141-B198) to stipulated glyceride(s) (e.g. those stipulated in B141-B198) is between 0:100 to 50:50 (i.e. the weight ratio is less than or equal to 50:50). Suitably, the weight ratio of unstipulated glyceride(s) (e.g. those other than the glyceride(s) stipulated in B141-B198) to stipulated glyceride(s) (e.g. those stipulated in B141-B198) is between 0:100 to 30:70 (i.e. the weight ratio is less than or equal to 30:70). Suitably, the weight ratio of unstipulated glyceride(s) (e.g. those other than the glyceride(s) stipulated in B141-B198) to stipulated glyceride(s) (e.g. those stipulated in B141-B198) is between 0:100 to 20:80 (i.e. the weight ratio is less than or equal to 20:80). Suitably, the weight ratio of unstipulated glyceride(s) (e.g. those other than the glyceride(s) stipulated in B141-B198) to stipulated glyceride(s) (e.g. those stipulated in B141-B198) is between 0:100 to 15:85 (i.e. the weight ratio is less than or equal to 15:85). Suitably, the weight ratio of unstipulated glyceride(s) (e.g. those other than the glyceride(s) stipulated in B141-B198) to stipulated glyceride(s) (e.g. those stipulated in B141-B198) is between 0:100 to 10:90 (i.e. the weight ratio is less than or equal to 10:90). Suitably, the weight ratio of unstipulated glyceride(s) (e.g. those other than the glyceride(s) stipulated in B141-B198) to stipulated glyceride(s) (e.g. those stipulated in B141-B198) is between 0:100 to 5:95 (i.e. the weight ratio is less than or equal to 5:95).

The one or more additional lipids may comprise one or more lipid(s) other than stipulated glyceride(s) (e.g. those stipulated in B141-B198), suitably where said one or more lipid(s) other than those stipulated may be referred to as “unstipulated” lipid(s). Suitably, the weight ratio of unstipulated lipid(s) (e.g. lipid(s) other than the glyceride(s) stipulated in B141-B198) to stipulated glyceride(s) (e.g. those stipulated in B141-B198) is between 0:100 to 50:50 (i.e. the weight ratio is less than or equal to 50:50). Suitably, the weight ratio of unstipulated lipid(s) (e.g. lipid(s) other than the glyceride(s) stipulated in B141-B198) to stipulated glyceride(s) (e.g. those stipulated in B141-B198) is between 0:100 to 30:70 (i.e. the weight ratio is less than or equal to 30:70). Suitably, the weight ratio of unstipulated lipid(s) (e.g. lipid(s) those other than the glyceride(s) stipulated in B141-B198) to stipulated glyceride(s) (e.g. those stipulated in B141-B198) is between 0:100 to 20:80 (i.e. the weight ratio is less than or equal to 20:80). Suitably, the weight ratio of unstipulated lipid(s) (e.g. lipid(s) those other than the glyceride(s) stipulated in B141-B198) to stipulated glyceride(s) (e.g. those stipulated in B141-B198) is between 0:100 to 15:85 (i.e. the weight ratio is less than or equal to 15:85). Suitably, the weight ratio of unstipulated lipid(s) (e.g. lipid(s) other than the glyceride(s) stipulated in B141-B198) to stipulated glyceride(s) (e.g. those stipulated in B141-B198) is between 0:100 to 10:90 (i.e. the weight ratio is less than or equal to 10:90). Suitably, the weight ratio of unstipulated lipid(s) (e.g. lipid(s) other than the glyceride(s) stipulated in B141-B198) to stipulated glyceride(s) (e.g. those stipulated in B141-B198) is between 0:100 to 5:95 (i.e. the weight ratio is less than or equal to 5:95).

### Additional Active

The one or more additional ingredients may comprise an additional active ingredient (e.g. in addition to the metal complex). Suitably, the additional active ingredient is other than a (different) metal complex (optionally with a different metal but the same drug, the same metal but a different drug, or a different drug and different metal).

### Additional Surfactant

The one or more additional ingredients may comprise a surfactant, especially for aqueous compositions (e.g. aqueous emulsions, aqueous dispersions, and aqueous solutions). Suitably, in the term “surfactant” excludes glyceride(s) or at least excludes any glyceride(s) defined/stipulated.

The surfactant may be a non-ionic surfactant. The non-ionic surfactant may be selected from the group consisting of: a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, polyglycerol esters, sucrose esters, saponins (e.g. saponin), and any combination thereof.

Suitably, the surfactant is selected from the group consisting of: polyglycerol esters, sucrose esters, saponins (e.g. saponin), and any combination thereof. Suitably, the surfactant is a saponin (e.g. saponin). In an embodiment, the surfactant is a poloxamer, most suitably poloxamer 407.

Suitably, where the composition comprises a surfactant, it does so on the proviso that the surfactant is not a PEG-based surfactant (e.g. the surfactant does not comprise polyethylene glycol polymers or copolymeric blocks of polyethylene glycol). Suitably, where the composition comprises a surfactant, it does so on the proviso that the surfactant is not a poloxamer. Suitably, where the composition comprises a surfactant, it does so on the proviso that the surfactant is not poloxamer 407. Suitably, where the composition comprises a surfactant, it does so on the proviso that the surfactant is not polysorbate 20. Suitably, where the composition comprises a surfactant, it does so on the proviso that the surfactant is not PEG-40 castor oil (e.g. not Killiphor RH 40). It is preferable to exclude or at least minimise the aforesaid surfactants.

Where the composition comprises a surfactant, suitably the surfactant is present (suitably where the composition comprises greater than or equal to 45 wt % water) at a concentration of 0.001-10 wt % of the overall composition. Where the composition comprises a surfactant, suitably the surfactant is present (suitably where the composition comprises greater than or equal to 45 wt % water) at a concentration of 0.01-2 wt % of the overall composition. Where the composition comprises a surfactant, suitably the surfactant is present (suitably where the composition comprises greater than or equal to 45 wt % water) at a concentration of 0.1-1.5 wt % of the overall composition. Where the composition comprises a surfactant, suitably the surfactant is present (suitably where the composition comprises greater than or equal to 45 wt % water) at a concentration of 0.2-0.6 wt % of the overall composition.

Suitably, the composition comprises at most 1 wt % surfactant(s), suitably at most 1 wt % of any surfactants defined in any of B223-B235 (especially poloxamer, polysorbate, and PEG-40 castor oil surfactants).

Suitably, the composition comprises at most 0.1 wt % surfactant(s), suitably at most 0.1 wt % of any surfactants defined in any of B223-B235 (especially poloxamer, polysorbate, and PEG-40 castor oil surfactants).

Suitably, the composition comprises at most 0.01 wt % surfactant(s), suitably at most 0.01 wt % of any surfactants defined in any of B223-B235 (especially poloxamer, polysorbate, and PEG-40 castor oil surfactants).

Suitably, the composition comprises at most 0.001 wt % surfactant(s), suitably at most 0.001 wt % of any surfactants defined in any of B223-B235 (especially poloxamer, polysorbate, and PEG-40 castor oil surfactants).

Suitably, the composition is free of surfactants, suitably free of any surfactants defined in any of B223-B235 (especially poloxamer, polysorbate, and PEG-40 castor oil surfactants).

### Additional Oils

The one or more additional ingredients comprise one or more oils. Such oil(s) may suitably comprise or consist of a natural oil. Where oils are present, most suitably the oils comprise or consist of an organic oil (suitably animal or vegetable oil). Suitably, the organic oil is a vegetable oil (usually rich in mono, di, and/or triglycerides) selected from the group consisting of (or any combination thereof):
Common vegetable oils: Coconut oil, Corn oil, Cottonseed oil, Olive oil, Palm oil, Peanut oil (Ground nut oil), Rapeseed oil (including Canola oil), Safflower oil, Sesame oil, Soybean oil, Sunflower oil; Nut oils: Almond oil, Beech nut oil, Brazil nut oil, Cashew oil, Hazelnut oil, Macadamia oil, Mongongo nut oil, Pecan oil, Pine nut oil, Pistachio oil, Walnut oil;
Citrus oils: Grapefruit seed oil, Lemon oil, Orange oil, Cucurbitaceae oils: Bitter gourd oil, Bottle gourd oil, Buffalo gourd oil, Butternut squash seed oil, Egusi seed oil, Pumpkin seed oil, Watermelon seed oil;
Food Supplement oils: Agai oil, Black seed oil, Blackcurrant seed oil, Borage seed oil, Evening primrose oil, Flaxseed oil;
Other Edible oils: Amaranth oil, Apricot oil, Apple seed oil, Argan oil, Avocado oil, Babassu oil, Ben oil, Borneo tallow nut oil, Cape chestnut oil, Carob pod oil (Algaroba oil)Cocoa butter, Cocklebur oil, Cohune oil, Coriander seed oil, Date seed oil, Dika oil, False flax oil, Grape seed oil, Hemp oil, Kapok seed oil, Kenaf seed oil, Lallemantia oil, Mafura oil, Marula oil, Meadowfoam seed oil, Mustard oil (pressed), Niger seed oil, Poppyseed oil, Nutmeg butter, Okra seed oil, Papaya seed oil, Perilla seed oil, Persimmon seed oil, Pequi oil, Pili nut oil, Pomegranate seed oil, Poppyseed oil, Pracaxi oil, Virgin pracaxi oil, Prune kernel oil, Quinoa oil, Ramtil oil, Rice bran oil, Royle oil, Shea nuts, Sacha inchi oil, Sapote oil, Seje oil, Shea butter, Taramira oil, Tea seed oil (Camellia oil), Thistle oil, Tigernut oil (or nut-sedge oil), Tobacco seed oil, Tomato seed oil, Wheat germ oil;
Multi-purpose oils: Castor oil, Coconut oil (copra oil), Colza oil, Corn oil, Cottonseed oil, False flax oil, Hemp oil, Mustard oil, Palm oil, Peanut oil, Radish oil, Rapeseed oil, Ramtil oil, Rice bran oil, Safflower oil, Salicornia oil, Soybean oil, Sunflower oil, Tigernut oil, Tung oil;
Inedible oils: Copaiba, Jatropha oil, Jojoba oil, Milk bush, Nahor oil, Paradise oil, Petroleum nut oil, Pongamia oil (also known as Honge oil);
Drying oils: Dammar oil, Linseed oil, Poppyseed oil, Stillingia oil, Tung oil, Vernonia oil; and Miscellaneous vegetable oils: Amur cork tree fruit oil, Artichoke oil, Astrocaryum murumuru butter, Balanos oil, Bladderpod oil, Brucea javanica oil, Burdock oil (Bur oil), Buriti oil, Candlenut oil (Kukui nut oil), Carrot seed oil (pressed), Castor oil, Chaulmoogra oil, Crambe oil, Croton oil (tiglium oil), Cuphea oil, Cupuagu butter, Honesty oil, Illipe butter, Jojoba oil, Mango oil, Mowrah butter, Neem oil, Ojon oil, Passion fruit oil, Rose hip seed oil, Rubber seed oil, Sea buckthorn oil, Sea rocket seed oil, Snowball seed oil (Viburnum oil), Tall oil, Tamanu or foraha oil, Tonka bean oil (Cumaru oil), Tucuma butter, Ucuhuba seed oil, moringa oil.

Suitably, the organic oil is a moringa oil, coconut oil, sunflower oil, avocado oil, or any combination thereof. As such, in a particular embodiment, the composition comprises a moringa oil, coconut oil, sunflower oil, avocado oil, or any combination thereof.

Where oil(s) are present, suitably the weight ratio of stipulated glyceride(s) to oil(s) is 99:1 to 30:70. Suitably, the weight ratio of stipulated glyceride(s) to oil(s) is 95:5 to 50:40. Suitably, the weight ratio of stipulated glyceride(s) to oil(s) is 90:10 to 60:40.

### Additional Bile Acids or Bile Salts

The one or more additional ingredients may comprise individual or mixtures of bile acids or bile salts. As such, in an embodiment, the composition comprises a mixture of bile salts (and/or bile acid(s)). Bile salts (and/or bile acid(s)) are particularly advantageous for digesting lipid-based systems of the invention in situ following absorption into the body or absorption upon/with a locality upon or within the body to which the composition is applied.

### Absent/Low-Level Ingredients

Preferably, the composition is (substantially, preferably entirely) free of polymers. The composition is preferably (substantially, preferably entirely) free of polyvinyl alcohol (PVA). Preferably, the composition is (substantially, preferably entirely) free of polyalkylene glycol(s) (e.g. polyethylene glycol and/or polypropylene glycol). Preferably, the composition is (substantially, preferably entirely) free of polyalkylene glycol ester(s) (e.g. polyethylene glycol and/or polypropylene glycol ester(s)). Preferably, the composition is (substantially, preferably entirely) free of any compounds that comprise polyalkylene glycol ester moieties (e.g. polyethylene glycol and/or polypropylene glycol ester moieties). Preferably, the composition is (substantially, preferably entirely) free of polyethylene glycol. Preferably, the composition is (substantially, preferably entirely) free of polyethylene glycol ester(s). Preferably, the composition is (substantially, preferably entirely) free of any compounds that comprise polyethylene glycol ester moieties.

Preferably, the composition is (substantially, preferably entirely) free of surfactants. Preferably, the composition is (substantially, preferably entirely) free of poloxamer surfactants. Preferably, the composition is (substantially, preferably entirely) free of poloxamer 407 (e.g. Pluronic F127). Preferably, the composition is (substantially, preferably entirely) free of polysorbate surfactants. Preferably, the composition is (substantially, preferably entirely) free of polysorbate 20. Preferably, the composition is (substantially, preferably entirely) free of PEG-40 castor oil (e.g. Killiphor RH 40, Cremophor RH40) surfactants. Preferably, the composition is (substantially, preferably entirely) free of poloxamer and polysorbate surfactants. Preferably, the composition is (substantially, preferably entirely) free of poloxamer. polysorbate, and PEG-40 castor oil surfactants. Preferably, the composition comprises at most 3 wt % surfactant(s), suitably at most 3 wt % of any surfactants defined in any of B262-B269 (especially poloxamer. polysorbate, and PEG-40 castor oil surfactants).

Preferably, the composition comprises at most 1 wt % surfactant(s), suitably at most 1 wt % of any surfactants defined in any of B262-B269 (especially poloxamer. polysorbate, and PEG-40 castor oil surfactants). Preferably, the composition comprises at most 0.1 wt % surfactant(s), suitably at most 0.1 wt % of any surfactants defined in any of B262-B269 (especially poloxamer. polysorbate, and PEG-40 castor oil surfactants). Preferably, the composition comprises at most 0.01 wt % surfactant(s), suitably at most 0.01 wt % of any surfactants defined in any of B262-B269 (especially poloxamer. polysorbate, and PEG-40 castor oil surfactants). Preferably, the composition comprises at most 0.001 wt % surfactant(s), suitably at most 0.001 wt % of any surfactants defined in any of B262-B269 (especially poloxamer. polysorbate, and PEG-40 castor oil surfactants).

Suitably, the composition is (substantially, preferably entirely) free of lipids other than those stipulated.

Preferably, the composition is (substantially, preferably entirely) free of solvents (i.e. compounds that are liquids at SATP) other than water.

Preferably, the composition is substantially (preferably entirely) free of organic solvents (suitably whether protic, polar, or unpolar).

### Other Composition-Specific Facets

Suitably, at least 1 wt % of the composition is constituted by the stipulated ingredients (i.e. those listed as being present or optionally present within the composition). Suitably, at least 10 wt % of the composition is constituted by the stipulated ingredients. Suitably, at least 15 wt % of the composition is constituted by the stipulated ingredients. Suitably, at least 20 wt % of the composition is constituted by the stipulated ingredients. Suitably, at least 50 wt % of the composition is constituted by the stipulated ingredients. Suitably, at least 70 wt % of the composition is constituted by the stipulated ingredients. Suitably, at least 80 wt % of the composition is constituted by the stipulated ingredients. Suitably, at least 85 wt % of the composition is constituted by the stipulated ingredients. Suitably, at least 90 wt % of the composition is constituted by the stipulated ingredients. Suitably, at least 95 wt % of the composition is constituted by the stipulated ingredients. Suitably, at least 99 wt % of the composition is constituted by the stipulated ingredients. Suitably, the composition consists of the stipulated ingredients.

Suitably, at least 1 wt % of the composition is constituted by the stipulated metal complex and stipulated glyceride(s) (e.g. as per the glyceride(s) stipulated in B141-B198). Suitably, at least 10 wt % of the composition is constituted by the stipulated metal complex and stipulated glyceride(s). Suitably, at least 15 wt % of the composition is constituted by the stipulated metal complex and stipulated glyceride(s). Suitably, at least 20 wt % of the composition is constituted by the stipulated metal complex and stipulated glyceride(s). Suitably, at least 50 wt % of the composition is constituted by the stipulated metal complex and stipulated glyceride(s). Suitably, at least 70 wt % of the composition is constituted by the stipulated metal complex and stipulated glyceride(s). Suitably, Suitably, at least 80 wt % of the composition is constituted by the stipulated metal complex and stipulated glyceride(s). Suitably, at least 85 wt % of the composition is constituted by the stipulated metal complex and stipulated glyceride(s). Suitably, at least 90 wt % of the composition is constituted by the stipulated metal complex and stipulated glyceride(s). Suitably, at least 95 wt % of the composition is constituted by the stipulated metal complex and stipulated glyceride(s). Suitably, at least 99 wt % of the composition is constituted by the stipulated metal complex and stipulated glyceride(s).

Suitably, centrifugation (e.g. 3000 rpm for 5 min at RT) of the composition yields less than 10 wt % sedimentation (i.e. less than 10 wt % of the composition as a whole). Suitably, centrifugation (e.g. 3000 rpm for 5 min at RT) of the composition yields less than 5 wt % sedimentation (i.e. less than 5 wt % of the composition as a whole). Suitably, centrifugation (e.g. 3000 rpm for 5 min at RT) of the composition yields less than 2 wt % sedimentation (i.e. less than 2 wt % of the composition as a whole). Suitably, centrifugation (e.g. 3000 rpm for 5 min at RT) of the composition yields less than 1 wt % sedimentation (i.e. less than 1 wt % of the composition as a whole). Suitably, centrifugation (e.g. 3000 rpm for 5 min at RT) of the composition yields less than 0.1 wt % sedimentation (i.e. less than 0.1 wt % of the composition as a whole). Suitably, centrifugation (e.g. 3000 rpm for 5 min at RT) of the composition yields less than 0.01 wt % sedimentation (i.e. less than 0.01 wt % of the composition as a whole). Suitably, centrifugation (e.g. 3000 rpm for 5 min at RT) of the composition yields (substantially) no sedimentation.

Suitably, centrifugation (e.g. 3000 rpm for 5 min at RT) of the composition yields either no sedimentation or sedimentation containing less than 50 wt % of the metal complex present within the composition (i.e. this refers to 50 wt % of the total amount of metal complex, not 50 wt % of the composition as a whole). Suitably, centrifugation (e.g. 3000 rpm for 5 min at RT) of the composition yields either no sedimentation or sedimentation containing less than 20 wt % of the metal complex present within the composition. Suitably, centrifugation (e.g. 3000 rpm for 5 min at RT) of the composition yields either no sedimentation or sedimentation containing less than 10 wt % of the metal complex present within the composition. Suitably, centrifugation (e.g. 3000 rpm for 5 min at RT) of the composition yields either no sedimentation or sedimentation containing less than 5 wt % of the metal complex present within the composition. Suitably, centrifugation (e.g. 3000 rpm for 5 min at RT) of the composition yields either no sedimentation or sedimentation containing less than 1 wt % of the metal complex present within the composition. Suitably, centrifugation (e.g. 3000 rpm for 5 min at RT) of the composition yields either no sedimentation or sedimentation containing less than 0.1 wt % of the metal complex present within the composition. Suitably, centrifugation (e.g. 3000 rpm for 5 min at RT) of the composition yields either no sedimentation of any metal complex present within the composition.

### Method of Preparing Composition

The present invention provides a method of preparing a composition (suitably as defined herein), said method being suitably as defined herein. The method suitably involves mixing together all of the ingredients defined in respect of the composition, including such compositions as defined hereinbefore and hereinafter, including in numbered paragraphs A1-A101, B1-B427, and combinations thereof. As such, the method suitably comprises mixing a metal complex with the one or more glycerides. Suitably, the metal complex is mixed with the one or more glycerides at a temperature at which the one or more glycerides are in a melted phase (e.g. 30-90° C., suitably 35-70° C., most preferably 40-45° C.), suitably for sufficient time to ensure homogenisation of the resultant composition. The composition may be optionally cooled or allowed to cool, and optionally stored at room temperature (e.g. 15-25° C., or SATP) or under refrigeration, (e.g. 1-8° C.). Such a method is particularly apt for preparing an unhydrated/dehydrated composition (which preferably has at most 5 wt % water), though in principle water may be added to the mixture, suitably whilst the glyceride(s) are in the melted phase.

Where a moderately-hydrated composition is desired (e.g. 10-60 wt % water), suitably this is prepared by a method comprising:
i) performing the aforesaid method to provide an unhydrated/dehydrated composition;
ii) optionally (if desired) adding further metal complex to the unhydrated/dehydrated composition to furnish an enriched composition;
iii) adding water (optionally to the enriched composition); and
iv) mixing to form a hydrated-lipid-based mixture, suitably at a temperature at which the one or more glycerides are in a melted phase (e.g. 30-90° C., suitably 35-70° C., most preferably 40-45° C.), suitably for sufficient time to ensure homogenisation of the hydrated-lipid-based mixture, and optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.);
wherein suitably step ii) is performed before step iii).

Where a very-hydrated composition (e.g. aqueous dispersion or aqueous emulsion) is desired (e.g. 85-99.9 wt % water), suitably this is prepared by a method comprising:
i) performing one of the aforesaid methods to provide either an unhydrated/dehydrated composition or a moderately hydrated composition;
ii) mixing the composition with the same weight or more of water (to provide a desired dilution), optionally in the presence of a surfactant (though suitably a surfactant as defined herein, suitably excluding certain surfactants), suitably at elevated temperatures (e.g. 30-90° C., suitably 35-70° C., most preferably 40-45° C.), optionally with ultra-sonication, to produce an emulsion or cubosome dispersion;
iii) optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.).

Any of the aforesaid methods may additionally comprise adding (or mixing in, preferably whilst the glycerides are in a melted phase) one or more additional ingredients (suitably as defined herein in relation to the composition).

The method may further comprise taking the product of the aforesaid method(s) (or a sample thereof) and subjecting it to centrifugation (e.g. 3000 rpm for 5 min at RT) to verify that one or more of the sedimentation requirements, defined herein, are met (e.g. as per B413-B427).

The present invention also provides a composition obtained by or directly obtained by the aforesaid method(s).

### Method of Preparing Other Products Using Composition

The present invention provides a product comprising a composition as defined herein. The product may suitably incorporate the composition.

The present invention also provides a method of preparing said product, the method comprising incorporating the composition within a product, suitably by mixing the composition with one or more additional ingredients (optionally as defined elsewhere herein) or one or more product ingredients (i.e. other ingredients used to make the product), or by incorporating the composition within an appropriate container or delivery device. For instance, in the case of a pharmaceutical product (e.g. a tablet), product ingredient(s) may include pharmaceutically acceptable excipient(s) or carrier(s). In the case the product is a spray, the composition may be mixed with one or more additional ingredients or product ingredients and incorporated within a spray device (e.g. having a reservoir, a spray nozzle, and a conduit connecting the contents of the reservoir with the spray nozzle).

The present invention also provides a product obtained by or directly obtained by the aforesaid method.

The product may suitably be (and most preferably is) a topical product (e.g. for use on a bodily surface, for example skin, body cavity, eye(s), hair). For example, the topical product may be a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical product that is a cream or ointment. In a particular embodiment, the product is a cream, an ointment, a foam, or a perfume (e.g. perfume, aftershave, cologne, or eau de toilette). In a particular embodiment, the product is a cream. In another embodiment, the product is an ointment.

In an alternative embodiment, the product may be an oral topical product, for example, a mouthwash, a rinse, an oral spray, a suspension, and/or a dental gel, suitably which is intended to be taken by mouth but are not intended to be ingested.

In an alternative embodiment, the product is an ingestible product (e.g. orally-administrable product). Such an ingestible product may be selected from a lozenge, a gel, a jelly, a pastille, a tablet, a capsule, a toffee, a nougat, a chewy candy, and/or a chewing gum. In a particular embodiment, the ingestible product is (or is otherwise incorporated within) a pastille, a gel, or a jelly. In another embodiment, the ingestible product comprises a gel component, gelling component, or gellable component. The gel component, gelling component, or gellable component is preferably animal- or plant-derived, most preferably plant-derived. In fact, most preferably, the product is free of any animal-derived components/ingredients.

The gel component, gelling component, or gellable component may comprise or consist of a polysaccharide or derivative thereof (e.g. x-carrageenan). The gel component, gelling component, or gellable component may comprise or consists of a gelatin, agar, and/or agarose. The ingestible product may be a liquid, suitably a suspension, a dispersion, an emulsion, or a solution, most preferably a dispersion or emulsion of the aforesaid composition. The ingestible product may be a drink/beverage comprising the aforesaid composition.

In an embodiment, the product is a pharmaceutical product. Such a pharmaceutical product is suitably for use as a medicament (or may otherwise be used in the manufacture of a medicament). In an embodiment, the product composition is for (or is for use in) bio/medicinal applications including antiulcer, anticancer, antimutagenic, antithrombotic and antifungal medications, microbial infections (e.g. composition as an antimicrobial composition), viral infections and fungal infections,

The product may be in the form of an emulsion, a lotion, a milk, a liquid, a solid, a cream, a gel, a mousse, an ointment, a paste, a serum, a stick, a spray, a tonic, an aerosol, a foam, and/or a pencil. The product may be an oil-in-water dispersion. The product may be a water-in-oil dispersion.

In an embodiment, the product is a personal care product. Said personal care product may be selected from the group consisting of skin care product, skin creams, shaving products (e.g. shaving creams, gels, foams), moisturizers, lotions, body washes, body oils, and hair care products (e.g. shampoos and/or hair conditioners). The personal care product may be a “leave-on-type” product (e.g. moisturizing lotions, serums, creams) or a “rinse-off-type” product (e.g. body washes, shampoos, hair conditioners, shower gels, skin cleansers, cleansing milks, shaving compositions). The personal care product may be in the form of an implement (e.g. a powdered or unpowdered cosmetic applicator), a reusable or disposable wipe, a tissue, a towel, a diaper, a razor or other shaving device, a personal cleansing implement (e.g. a mesh shower sponge), a conventional sponge, a wash cloth, a swab, and/or a pen.

In an embodiment, the product is a cosmetic product. The cosmetic product may be for controlling and/or reducing the formation of wrinkles and lines in the skin. The cosmetic product may be for providing glowing skin.

In an embodiment, the product is a wound care product. The wound care (or wound healing) product may be a wound care hydrogel composition, and therefor suitably comprises a hydrogel or hydrogel-forming component or compound—this may be a polysaccharide-based hydrogel or hydrogel-forming component or compound. Such a wound care product may be selected from the group consisting of a wound dressing, a sutures, a staple, a gauze, a bandage, a plaster, a burn dressing, an artificial skin, a liposome or micelle formulation, a microcapsule, an aqueous vehicle for soaking gauze dressings. In an embodiment, the wound care product may be for (or for use in) healing wounds, for example, healing cuts and scrapes, burns, ulcers, bed sores, fissures, haemorrhoids, and/or post-surgical wounds. The same properties that render compositions of the invention excellent for topical applications also apply to wound care products.

The product may be a textile—e.g. a textile (e.g. a clothing item) comprising the aforesaid composition.

### Uses of Composition and Products Formed Therefrom

Compositions and products of the invention may be used in a variety of ways, as defined herein.

By way of example, the present invention provides a use of a composition (as defined herein) or product (as defined herein) for topical application to bodily surfaces (e.g. skin, eye(s), hair, body cavity), for topical application to the mouth, for oral administration/ingestion. Sub-definitions of such uses will be readily apparent from recitations of these general categories throughout this specification—for instance, paragraphs B2-B41 elucidate such uses further.

The present invention also provides a use of a composition (as defined herein) or product (as defined herein) for pharmaceutical treatments, for cosmetic treatments/applications, for dental treatments, for eye treatments, for personal care (e.g. skin care, hair care), for wound care. Again, sub-definitions of all such uses will be readily apparent from recitations of these general categories throughout this specification—for instance, paragraphs B2-B41 elucidate such uses further.

### Medical Uses

The present invention provides a composition (e.g. a pharmaceutical composition), suitably as defined herein, or a product (e.g. pharmaceutical product), suitably as defined herein, for use as a medicament (or for use in treating a disease, disorder, or medical condition, suitably a disease, disorder, or medical condition as defined herein).

The present invention provides a use of a composition (e.g. a pharmaceutical composition), suitably as defined herein, or a product (e.g. pharmaceutical product), suitably as defined herein, in the manufacture of a medicament (or in the manufacture of a medicament for treating a disease, disorder, or medical condition, suitably a disease, disorder, or medical condition as defined herein).

The present invention provides a method of treating a disease, disorder, or medical condition (suitably a disease, disorder, or medical condition as defined herein) in a subject (suitably in a subject identified as in need of treatment), the method comprising administering to the subject a therapeutically effective amount of a composition (e.g. a pharmaceutical composition), suitably as defined herein, or a product (e.g. pharmaceutical product), suitably as defined herein.

Suitably, for (or is for use in) bio/medicinal applications including antiulcer, anticancer, antimutagenic, antithrombotic and antifungal medications, microbial infections (e.g. composition as an antimicrobial composition), viral infections and fungal infections. It is also suitable to treat rheumatoid arthritis and similar disorders.

Suitably, administering the composition or the product to the subject comprises topical administration or oral administration, most preferably topical administration.

### Specific Embodiments

The examples and data presented herein suggest that certain technical effects may arise from certain features or feature combinations, in light of which various specific embodiments A1-A101 are discussed below. A person skilled in the art will readily appreciate embodiments elucidated by the dependencies set forth below, including via direct and indirect dependencies. The embodiments disclosed in this section may be combined with any embodiments and features disclosed in any other section herein.

### A) Specific Embodiments

A1. A composition comprising a metal complex (preferably a copper-drug complex, suitably copper aspirinate or copper salicylate) and one or more glycerides (which, together with any extra, are ingredients of the composition), wherein at least one (preferably each) of the glyceride(s) comprises at least one unsaturated moiety (preferably at least one alkene moiety, most preferably at least one cis-alkene moiety), suitably wherein the composition comprises at most 10 wt % water (preferably at most 5 wt % water, most preferably at most 2 wt % water).
A2. A composition comprising a metal complex (preferably a copper-drug complex, suitably copper aspirinate or copper salicylate), one or more glycerides, and water (which, together with any extra, are ingredients of the composition), wherein at least one (preferably each) of the glyceride(s) comprises at least one unsaturated moiety (preferably at least one alkene moiety, most preferably at least one cis-alkene moiety).
A3. A method of preparing the composition of A1, the method comprising mixing the metal complex (preferably a copper-drug complex, suitably copper aspirinate or copper salicylate) with the one or more glycerides (i.e. mixing the ingredients of the composition, which may include additional ingredients, such as those stipulated hereinafter in relation to the composition) to form a lipid-based mixture, suitably at a temperature at which the one or more glycerides are in a melted phase (e.g. 30-90° C., suitably 35-70° C., most preferably 40-45° C.), suitably for sufficient time to ensure homogenisation of the lipid-based mixture, and optionally cooling the lipid-based mixture (or allowing the lipid-based mixture to cool) and optionally thereafter storing (e.g. at room temperature, e.g. 15-25° C. or under refrigeration, e.g. 1-8° C.).
A4. A method of preparing the composition of A2, the method comprising:
   i) performing the method of A3 or otherwise providing the composition of A1;
   ii) adding further metal complex (preferably a copper-drug complex, suitably copper aspirinate or copper salicylate) to the composition;
   iii) adding water (now a further ingredient of the composition) to the composition;
   iv) mixing to form a hydrated-lipid-based mixture, suitably at a temperature at which the one or more glycerides are in a melted phase (e.g. 30-90° C., suitably 35-70° C., most preferably 40-45° C.), suitably for sufficient time to ensure homogenisation of the hydrated-lipid-based mixture, and optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.);
wherein suitably step ii) is performed before step iii).
A5. A method of preparing the composition of A2, the method comprising:
   i) performing the method of A3 or A4 or otherwise providing the composition of A1 or A2;
   ii) mixing the composition with the same weight or more of water (to provide a desired dilution), optionally in the presence of a surfactant (though suitably a surfactant as defined herein, suitably excluding certain surfactants), suitably at elevated temperatures (e.g. 30-90° C., suitably 35-70° C., most preferably 40-45° C.), optionally with ultra-sonication, to produce an emulsion or cubosome dispersion;
   iii) optionally cooling the hydrated-lipid-based mixture (or allowing the hydrated-lipid-based mixture to cool) and optionally thereafter storing (e.g. at room temperature, e.g. 15-25° C. or under refrigeration, e.g. 1-8° C.).
A6. The method of any of A3-A5, wherein the method comprises adding (or mixing in) one or more additional ingredients.
A7. A product comprising either the composition of A1-A2 or a composition obtained by (or directly obtained by) the method of A3-A5.
A8. A method of preparing the product of A7, the method comprising incorporating the composition of A1-A2 or a composition obtained by (or directly obtained by) the method of A3-A5 within a product, suitably by mixing the composition with one or more additional ingredients (optionally as defined elsewhere herein) or one or more product ingredients (i.e. other ingredients used to make the product), or by incorporating the composition within an appropriate container or delivery device.
A9. A composition (e.g. a pharmaceutical composition) of A1-A2 or a composition obtained by (or directly obtained by) the method of A3-A5, or a product (e.g. pharmaceutical product) of A7 or product obtained (or directly obtained by) the method of A8, for use as a medicament (or for use in treating a disease, disorder, or medical condition, suitably a disease, disorder, or medical condition as defined herein).

A10. Use of a composition (e.g. a pharmaceutical composition) of A1-A2 or a composition obtained by (or directly obtained by) the method of A3-A5, or a product (e.g. pharmaceutical product) of A7 or product obtained (or directly obtained by) the method of A8, in the manufacture of a medicament (or in the manufacture of a medicament for treating a disease, disorder, or medical condition, suitably a disease, disorder, or medical condition as defined herein).

A11. A method of treating a disease, disorder, or medical condition (suitably a disease, disorder, or medical condition as defined herein) in a subject (suitably in a subject identified as in need of treatment), the method comprising administering to the subject a therapeutically effective amount of the composition (e.g. a pharmaceutical composition) of A1-A2 or a composition obtained by (or directly obtained by) the method of A3-A5, or a product (e.g. pharmaceutical product) of A7 or product obtained (or directly obtained by) the method of A8.
A12. The composition, method, product, or use of any of A1-A11, wherein the one or more glycerides are one or more monoglycerides bearing a C₈-C₂₀ fatty acid moiety having at least one cis-alkene.
A13. The composition, method, product, or use of A12, wherein the composition (i.e. ingredients of the composition) comprises a metal complex (preferably a copper-drug complex, suitably copper aspirinate or copper salicylate), one or more monoglycerides bearing a C₈-C₂₀ fatty acid moiety having at least one cis-alkene, and bile salts (and/or bile acid(s)).
A14. The composition, method, product, or use of A12, wherein the composition (i.e. ingredients of the composition) comprises a metal complex (preferably a copper-drug complex, suitably copper aspirinate or copper salicylate), glyceryl monooleate, glyceryl monolinoleate, and bile salts (and/or bile acid(s)).
A15. The composition, method, product, or use of any of A12-A13, wherein the composition (i.e. ingredients of the composition) comprises the metal complex (preferably a copper-drug complex, suitably copper aspirinate or copper salicylate), a first monoglyceride, and a second monoglyceride, wherein both the first monoglyceride and second monoglyceride are compounds comprising at least one alkene moiety (most preferably at least one cis-alkene moiety), wherein suitably the first monoglyceride and second monoglyceride are present in a weight ratio between 20:80 and 40:60.
A16. The composition, method, product, or use of A15, wherein the composition comprises a metal complex (preferably a copper-drug complex, suitably copper aspirinate or copper salicylate), glyceryl monooleate, and glyceryl monolinoleate, wherein suitably glyceryl monooleate and glyceryl monolinoleate are present in a weight ratio between 20:80 and 40:60.
A17. The composition, method, product, or use of A15, wherein the composition comprises a metal complex (preferably a copper-drug complex, suitably copper aspirinate or copper salicylate), glyceryl monooleate, and glyceryl monolinoleate, wherein suitably glyceryl monooleate and glyceryl monolinoleate are present in a weight ratio between 20:80 and 40:60.
A18. The composition, method, product, or use of A15, wherein the composition comprises a metal complex (preferably a copper-drug complex, suitably copper aspirinate or copper salicylate), glyceryl monooleate, glyceryl monolinoleate, and water, wherein water is at a concentration within the composition of 10-60 wt %, wherein suitably glyceryl monooleate and glyceryl monolinoleate are present in a weight ratio between 20:80 and 40:60.
A19. The composition, method, product, or use of A15, wherein the composition comprises a metal complex (preferably a copper-drug complex, suitably copper aspirinate or copper salicylate), glyceryl monooleate, glyceryl monolinoleate, water, and bile salts (and/or bile acid(s)), wherein water is at a concentration within the composition of 10-60 wt %, wherein suitably glyceryl monooleate and glyceryl monolinoleate are present in a weight ratio between 20:80 and 40:60.
A20. The composition, method, product, or use of A1-A19, wherein the composition comprises a metal complex (preferably a copper-drug complex, suitably copper aspirinate or copper salicylate), glyceryl monooleate, glyceryl monolinoleate, and bile salts (and/or bile acid(s)), wherein suitably glyceryl monooleate and glyceryl monolinoleate are present in a weight ratio between 20:80 and 40:60.
A21. The composition, method, product, or use of any of A1-A11, wherein the composition comprises a metal complex (preferably a copper-drug complex, suitably copper aspirinate or copper salicylate), and one or more monoglycerides selected from glyceryl monooleate, glyceryl monolinoleate, or any combination thereof, wherein the weight ratio of the metal complex to the glyceride(s) is 0.1:1000-700:1000.
A22. The composition, method, product, or use of any of A1-A11, wherein the composition comprises 0.001-40 wt % metal complex (preferably a copper-drug complex, suitably copper aspirinate or copper salicylate), and one or more monoglycerides bearing a C₈-C₂₀ fatty acid moiety having at least one cis-alkene, wherein the weight ratio of the metal complex to the one or more monoglyceride(s) is 0.1:1000-700:1000.
A23. The composition, method, product, or use of any of A1-A11, wherein the composition comprises a metal complex (preferably a copper-drug complex, suitably copper aspirinate or copper salicylate) and glyceryl monooleate, wherein: the composition has at most 5 wt % water.
A24. The composition, method, product, or use of any of A1-A11, wherein the composition comprises a metal complex (preferably a copper-drug complex, suitably copper aspirinate or copper salicylate), and glyceryl monooleate, wherein: the composition has at most 5 wt % water; and at least 20 wt % of the composition is constituted by the metal complex, glyceryl monooleate, and water (to the extent that any water is present), wherein the weight ratio of the metal complex to the glyceride(s) is 0.001:1000-10:1000.
A25. The composition, method, product, or use of any of A1-A11, wherein the composition comprises a metal complex (preferably a copper-drug complex, suitably copper aspirinate or copper salicylate) and glyceryl monooleate, wherein: the weight ratio of metal complex to glyceryl monooleate is 0.1:1000-5:1000; the composition has at most 5 wt % water; and at least 20 wt % of the composition is constituted by the metal complex, glyceryl monooleate, and water (to the extent that any water is present).
A26. The composition, method, product, or use of any of A1-A11, wherein the composition comprises a metal complex (preferably a copper-drug complex, suitably copper aspirinate or copper salicylate), and glyceryl monooleate, wherein: the composition has at most 2 wt % water; and at least 80 wt % of the composition is constituted by the metal complex, glyceryl monooleate, and water (to the extent that any water is present), wherein the weight ratio of the metal complex to the glyceride(s) is 0.1:1000-5:1000.
A27. The composition, method, product, or use of any of A1-A11, wherein the composition comprises a metal complex (preferably a copper-drug complex, suitably copper aspirinate or copper salicylate) and glyceryl monooleate, wherein: the weight ratio of metal complex to glyceryl monooleate is between 0.1:1000-5:1000; the composition has at most 2 wt % water; and at least 80 wt % of the composition is constituted by the metal complex, glyceryl monooleate, and water (to the extent that any water is present).
A28. The composition, method, product, or use of any of A1-A11, wherein the composition comprises a metal complex (preferably a copper-drug complex, suitably copper aspirinate or copper salicylate), and glyceryl monooleate, wherein: the composition has at most 2 wt % water; at least 80 wt % of the composition is constituted by the metal complex, glyceryl monooleate, and water (to the extent that any water is present); the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition, wherein the weight ratio of the metal complex to the glyceryl monooleate is 0.1:1000-5:1000.
A29. The composition, method, product, or use of any of A1-A11, wherein the composition comprises a metal complex (preferably a copper-drug complex, suitably copper aspirinate or copper salicylate) and glyceryl monooleate, wherein: the weight ratio of metal complex to glyceryl monooleate is between 0.1:1000-5:1000; the composition has at most 2 wt % water; at least 80 wt % of the composition is constituted by the metal complex, glyceryl monooleate, and water (to the extent that any water is present); the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition.
A30. The composition, method, product, or use of any of A1-A11, wherein the composition comprises a metal complex (preferably a copper-drug complex, suitably copper aspirinate or copper salicylate) and glyceryl monooleate, wherein: the composition comprises 10-60 wt % water.
A31. The composition, method, product, or use of any of A1-A11, wherein the composition comprises a metal complex (preferably a copper-drug complex, suitably copper aspirinate or copper salicylate) and glyceryl monooleate, wherein: the weight ratio of metal complex to glyceryl monooleate is 1:1000-200:1000; the composition comprises 10-60 wt % water; and at least 20 wt % of the composition is constituted by the metal complex, glyceryl monooleate, and water.
A32. The composition, method, product, or use of any of A1-A11, wherein the composition comprises a metal complex (preferably a copper-drug complex, suitably copper aspirinate or copper salicylate) and glyceryl monooleate, wherein: the weight ratio of metal complex to glyceryl monooleate is 20:1000-130:1000; the composition comprises 20-45 wt % water; and at least 80 wt % of the composition is constituted by the metal complex, glyceryl monooleate, and water.
A33. The composition, method, product, or use of any of A1-A11, wherein the composition comprises a metal complex (preferably a copper-drug complex, suitably copper aspirinate or copper salicylate) and glyceryl monooleate, wherein: the weight ratio of metal complex to glyceryl monooleate is 20:1000-130:1000; the composition comprises 20-45 wt % water; at least 80 wt % of the composition is constituted by the metal complex, glyceryl monooleate, and water; the composition is free of surfactants; the weight ratio of unstipulated glyceride(s) (e.g. those other than glyceryl monooleate) to glyceryl monooleate is between 0:100 to 15:85 (i.e. the weight ratio is less than or equal to 15:85), wherein glyceride(s) (i.e. glyceryl monooleate and any unstipulated glyceride(s)) constitute at least 95 wt % of the total amount of lipids present within the composition.
A34. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the composition has at most 5 wt % water.
A35. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 0.1:1000-5:1000; the composition has at most 5 wt % water; and at least 20 wt % of the composition is constituted by the metal complex, either or both glyceryl monooleate and/or glyceryl monolinoleate, and water (to the extent that any water is present).
A36. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate wherein: the weight ratio of metal complex to glyceryl monooleate and glyceryl monolinoleate combined is 0.1:1000-5:1000; the composition has at most 2 wt % water; and at least 80 wt % of the composition is constituted by the metal complex, either or both glyceryl monooleate and/or glyceryl monolinoleate, and water (to the extent that any water is present).
A37. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 0.1:1000-5:1000; the composition has at most 2 wt % water; at least 80 wt % of the composition is constituted by the metal complex, either or both glyceryl monooleate and/or glyceryl monolinoleate, and water (to the extent that any water is present); the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. either or both glyceryl monooleate and/or glyceryl monolinoleate, and any unstipulated glyceride(s)) constitute at least 95 wt % of the total amount of lipids present within the composition.
A38. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the composition comprises 10-60 wt % water.
A39. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 1:1000-200:1000; the composition comprises 10-60 wt % water; and at least 20 wt % of the composition is constituted by the metal complex, either or both glyceryl monooleate and/or glyceryl monolinoleate, and water.
A40. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 20:1000-130:1000; the composition comprises 20-45 wt % water; and at least 80 wt % of the composition is constituted by the metal complex, either or both glyceryl monooleate and/or glyceryl monolinoleate, and water.
A41. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate wherein: the composition comprises glyceryl monooleate and glyceryl monolinoleate in a weight ratio between 25:75 and 35:65; the weight ratio of metal complex to glyceryl monooleate and glyceryl monolinoleate combined (i.e. relative to total weight of glyceryl monooleate and glyceryl monolinoleate combined) is 20:1000-130:1000; the composition comprises 20-45 wt % water; at least 80 wt % of the composition is constituted by the metal complex, either or both glyceryl monooleate and/or glyceryl monolinoleate, and water; the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. either or both glyceryl monooleate and/or glyceryl monolinoleate, and any unstipulated glyceride(s)) constitute at least 95 wt % of the total amount of lipids present within the composition.
A42. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 10:1000-400:1000; the composition has at most 5 wt % water; and at least 20 wt % of the composition is constituted by the metal complex, either or both glyceryl monooleate and/or glyceryl monolinoleate, and water (to the extent that any water is present).
A43. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate wherein: the weight ratio of metal complex to glyceryl monooleate and glyceryl monolinoleate combined is 50:1000-200:1000; the composition has at most 2 wt % water; and at least 80 wt % of the composition is constituted by the metal complex, either or both glyceryl monooleate and/or glyceryl monolinoleate, and water (to the extent that any water is present).
A44. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 50:1000-200:1000; the composition has at most 2 wt % water; at least 80 wt % of the composition is constituted by the metal complex, either or both glyceryl monooleate and/or glyceryl monolinoleate, and water (to the extent that any water is present); the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. either or both glyceryl monooleate and/or glyceryl monolinoleate, and any unstipulated glyceride(s)) constitute at least 95 wt % of the total amount of lipids present within the composition.
A45. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 50:1000-700:1000; the composition comprises 10-60 wt % water; and at least 20 wt % of the composition is constituted by the metal complex, either or both glyceryl monooleate and/or glyceryl monolinoleate, and water.
A46. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 200:1000-580:1000; the composition comprises 20-45 wt % water; and at least 80 wt % of the composition is constituted by the metal complex, either or both glyceryl monooleate and/or glyceryl monolinoleate, and water.
A47. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate wherein: the composition comprises glyceryl monooleate and glyceryl monolinoleate in a weight ratio between 25:75 and 35:65; the weight ratio of metal complex to glyceryl monooleate and glyceryl monolinoleate combined (i.e. relative to total weight of glyceryl monooleate and glyceryl monolinoleate combined) is 200:1000-580:1000; the composition comprises 20-45 wt % water; at least 80 wt % of the composition is constituted by the metal complex, either or both glyceryl monooleate and/or glyceryl monolinoleate, and water; the composition is free of surfactants; the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. either or both glyceryl monooleate and/or glyceryl monolinoleate, and any unstipulated glyceride(s)) constitute at least 95 wt % of the total amount of lipids present within the composition.
A48. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the composition comprises at least 85 wt % water.
A49. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 1:1000-200:1000; the composition comprises at least 85 wt % water; and the composition is free of surfactants (or is free of free of poloxamer. polysorbate, and PEG-40 castor oil surfactants) or comprises at most 3 wt % surfactant(s) (or at most 3 wt % free of poloxamer. polysorbate, and PEG-40 castor oil surfactants).
A50. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate, wherein: the weight ratio of metal complex to glyceryl monooleate and glyceryl monolinoleate combined is 5:1000-100:1000; the composition comprises at least 85 wt % water; and the composition is free of surfactants (or is free of free of poloxamer. polysorbate, and PEG-40 castor oil surfactants) or comprises at most 3 wt % surfactant(s) (or at most 3 wt % free of poloxamer. polysorbate, and PEG-40 castor oil surfactants).
A51. The composition, method, product, or use of any of A1-A11, wherein the composition comprises a metal complex (preferably a copper-drug complex, suitably copper aspirinate or copper salicylate), either or both (preferably both) glyceryl monooleate and/or glyceryl monolinoleate wherein: the composition comprises glyceryl monooleate and glyceryl monolinoleate in a weight ratio between 25:75 and 35:65; the weight ratio of metal complex to glyceryl monooleate and glyceryl monolinoleate combined (i.e. relative to total weight of glyceryl monooleate and glyceryl monolinoleate combined) is 5:1000-100:1000; the composition comprises at least 85 wt % water; the composition is free of surfactants (or is free of free of poloxamer. polysorbate, and PEG-40 castor oil surfactants) or comprises at most 1 wt % surfactant(s) (or at most 1 wt % free of poloxamer. polysorbate, and PEG-40 castor oil surfactants); the weight ratio of unstipulated glyceride(s) (e.g. those other than glyceryl monooleate and glyceryl monolinoleate) to glyceryl monooleate and glyceryl monolinoleate combined is between 0:100 to 15:85 (i.e. the weight ratio is less than or equal to 15:85), wherein glyceride(s) (i.e. either or both glyceryl monooleate and/or glyceryl monolinoleate, and any unstipulated glyceride(s)) constitute at least 95 wt % of the total amount of lipids present within the composition.
A52. The composition, method, product, or use of any of A1-A11, wherein the composition comprises 0.001-0.1 wt % metal complex (preferably a copper-drug complex, suitably copper aspirinate or copper salicylate) and 50-99.999 wt % of the one or more glyceride(s).
A53. The composition, method, product, or use of any of A1-A11, wherein the composition consists of or consists essentially of 0.001-0.1 wt % metal complex (preferably a copper-drug complex, suitably copper aspirinate or copper salicylate) and 50-99.999 wt % of the one or more glyceride(s).
A54. The composition, method, product, or use of any of A1-A11, wherein the composition comprises 0.001-0.1 wt % metal complex (preferably a copper-drug complex, suitably copper aspirinate or copper salicylate) and 50-99.999 wt % glyceryl monooleate.
A55. The composition, method, product, or use of any of A1-A11, wherein the composition consists of or consists essentially of 0.001-0.1 wt % metal complex (preferably a copper-drug complex, suitably copper aspirinate or copper salicylate) and 50-99.999 wt % glyceryl monooleate.
A56. The composition, method, product, or use of any of A1-A11, wherein the composition comprises 0.001-0.1 wt % metal complex (preferably a copper-drug complex, suitably copper aspirinate or copper salicylate) and 50-99.999 wt % mixture of glyceryl monooleate and glyceryl monolinoleate in a respective weight ratio between 25:75 and 35:65.
A57. The composition, method, product, or use of any of A1-A11, wherein the composition consists of or consists essentially of 0.001-0.1 wt % metal complex (preferably a copper-drug complex, suitably copper aspirinate or copper salicylate) and 50-99.999 wt % mixture of glyceryl monooleate and glyceryl monolinoleate in a respective weight ratio between 25:75 and 35:65.
A58. The composition, method, product, or use of any of A1-A11, wherein the composition comprises 1-30 wt % (preferably 2-20 wt %) metal complex (preferably a copper-drug complex, suitably copper aspirinate or copper salicylate) and 10-99 wt % (preferably 50-95 wt %) of the one or more (preferably at least two, preferably two) glyceride(s).
A59. The composition, method, product, or use of any of A1-A11, wherein the composition consists of or consists essentially of 1-30 wt % (preferably 2-20 wt %) metal complex (preferably a copper-drug complex, suitably copper aspirinate or copper salicylate) and 10-99 wt % (preferably 50-95 wt %) of the one or more (preferably at least two, preferably two) glyceride(s).
A60. The composition, method, product, or use of any of A1-A11, wherein the composition comprises 1-30 wt % (preferably 2-20 wt %) metal complex (preferably a copper-drug complex, suitably copper aspirinate or copper salicylate) and 10-99 wt % (preferably 50-95 wt %) of the one or more (preferably at least two, preferably two) glyceride(s).
A61. The composition, method, product, or use of any of A1-A11, wherein the composition consists of or consists essentially of 1-30 wt % (preferably 2-20 wt %) metal complex (preferably a copper-drug complex, suitably copper aspirinate or copper salicylate) and 10-99 wt % (preferably 50-95 wt %) of the one or more (preferably at least two, preferably two) glyceride(s).
A62. The composition, method, product, or use of any of A1-A11, wherein the composition comprises 1-30 wt % (preferably 2-20 wt %) metal complex (preferably a copper-drug complex, suitably copper aspirinate or copper salicylate) and 10-99 wt % (preferably 50-95 wt %) mixture of glyceryl monooleate and glyceryl monolinoleate in a respective weight ratio between 25:75 and 35:65.
A63. The composition, method, product, or use of any of A1-A11, wherein the composition consists of or consists essentially of 1-30 wt % (preferably 2-20 wt %) metal complex (preferably a copper-drug complex, suitably copper aspirinate or copper salicylate) and 90-99 wt % (preferably 90-95 wt %) mixture of glyceryl monooleate and glyceryl monolinoleate in a respective weight ratio between 25:75 and 35:65.
A64. The composition, method, product, or use of any of A1-A11, wherein the composition comprises 0.1-15 wt % (preferably 1-10 wt %) metal complex (preferably a copper-drug complex, suitably copper aspirinate or copper salicylate), 30-90 wt % (preferably 40-80 wt %) of the one or more glyceride(s), and 15-60 wt % (preferably 25-50 wt %) water.
A65. The composition, method, product, or use of any of A1-A11, wherein the composition consists of or consists essentially of 0.1-15 wt % (preferably 1-10 wt %) metal complex (preferably a copper-drug complex, suitably copper aspirinate or copper salicylate), 30-90 wt % (preferably 40-80 wt %) of the one or more glyceride(s), and 15-60 wt % (preferably 25-50 wt %) water.
A66. The composition, method, product, or use of any of A1-A11, wherein the composition comprises 0.1-15 wt % metal complex (preferably a copper-drug complex, suitably copper aspirinate or copper salicylate), 30-90 wt % either or both (preferably both) glyceryl monooleate and/or glyceryl monolinoleate (in a respective weight ratio between 25:75 and 35:65 when both), and 15-60 wt % water.
A67. The composition, method, product, or use of any of A1-A11, wherein the composition comprises 1-10 wt % metal complex (preferably a copper-drug complex, suitably copper aspirinate or copper salicylate), 40-80 wt % either or both (preferably both) glyceryl monooleate and/or glyceryl monolinoleate (in a respective weight ratio between 25:75 and 35:65 when both), and 25-50 wt % water.
A68. The composition, method, product, or use of any of A1-A11, wherein the composition consists of or consists essentially of 1-10 wt % metal complex (preferably a copper-drug complex, suitably copper aspirinate or copper salicylate), 40-80 wt % either or both (preferably both) glyceryl monooleate and/or glyceryl monolinoleate (in a respective weight ratio between 25:75 and 35:65 when both), and 25-50 wt % water.
A69. The composition, method, product, or use of any of A1-A11, wherein the composition comprises 1-10 wt % (preferably 2-7 wt %) metal complex (preferably a copper-drug complex, suitably copper aspirinate or copper salicylate), 40-80 wt % (preferably 50-70 wt %) of the one or more (preferably at least two, preferably two) glyceride(s), and 25-50 wt % (preferably 30-45 wt %) water.
A70. The composition, method, product, or use of any of A1-A11, wherein the composition consists of or consists essentially of 1-10 wt % (preferably 2-7 wt %) metal complex (preferably a copper-drug complex, suitably copper aspirinate or copper salicylate), 40-80 wt % (preferably 50-70 wt %) of the one or more (preferably at least two, preferably two) glyceride(s), and 25-50 wt % (preferably 30-45 wt %) water.
A71. The composition, method, product, or use of any of A1-A11, wherein the composition comprises 1-10 wt % (preferably 2-7 wt %) metal complex (preferably a copper-drug complex, suitably copper aspirinate or copper salicylate), 40-80 wt % (preferably 50-70 wt %) mixture of glyceryl monooleate and glyceryl monolinoleate in a respective weight ratio between 25:75 and 35:65, and 25-50 wt % (preferably 30-45 wt %) water.
A72. The composition, method, product, or use of any of A1-A11, wherein the composition consists of or consists essentially of 1-10 wt % (preferably 2-7 wt %) metal complex (preferably a copper-drug complex, suitably copper aspirinate or copper salicylate), 40-80 wt % (preferably 50-70 wt %) mixture of glyceryl monooleate and glyceryl monolinoleate in a respective weight ratio between 25:75 and 35:65, and 25-50 wt % (preferably 30-45 wt %) water.
A73. The composition, method, product, or use of any of A1-A11, wherein the composition comprises 5-40 wt % (preferably 20-30 wt %) metal complex (preferably a copper-drug complex, suitably copper aspirinate or copper salicylate), 20-80 wt % (preferably 40-50 wt %) of the one or more (preferably at least two, preferably two) glyceride(s), and 10-50 wt % (preferably 25-35 wt %) water.
A74. The composition, method, product, or use of any of A1-A11, wherein the composition consists of or consists essentially of 5-40 wt % (preferably 20-30 wt %) metal complex (preferably a copper-drug complex, suitably copper aspirinate or copper salicylate), 20-80 wt % (preferably 40-50 wt %) of the one or more (preferably at least two, preferably two) glyceride(s), and 10-50 wt % (preferably 25-35 wt %) water.
A75. The composition, method, product, or use of any of A1-A11, wherein the composition comprises 5-40 wt % (preferably 20-30 wt %) metal complex (preferably a copper-drug complex, suitably copper aspirinate or copper salicylate), 20-80 wt % (preferably 40-50 wt %) mixture of glyceryl monooleate and glyceryl monolinoleate in a respective weight ratio between 25:75 and 35:65, and 10-50 wt % (preferably 25-35 wt %) water.
A76. The composition, method, product, or use of any of A1-A11, wherein the composition consists of or consists essentially of 5-40 wt % (preferably 20-30 wt %) metal complex (preferably a copper-drug complex, suitably copper aspirinate or copper salicylate), 20-80 wt % (preferably 40-50 wt %) mixture of glyceryl monooleate and glyceryl monolinoleate in a respective weight ratio between 25:75 and 35:65, and 10-50 wt % (preferably 25-35 wt %) water.
A77. The composition, method, product, or use of any of A1-A11, wherein the composition comprises 0.001-5 wt % (preferably 0.01-1 wt %) metal complex (preferably a copper-drug complex, suitably copper aspirinate or copper salicylate), 0.1-20 wt % (preferably 1-10 wt %) of the one or more (preferably at least two, preferably two) glyceride(s), and 85-99.9 wt % (preferably 90-97 wt %) water.
A78. The composition, method, product, or use of any of A1-A11, wherein the composition comprises 0.001-5 wt % (preferably 0.01-1 wt %) metal complex (preferably a copper-drug complex, suitably copper aspirinate or copper salicylate), 0.1-20 wt % (preferably 1-10 wt %) of the one or more (preferably at least two, preferably two) glyceride(s), 85-99.9 wt % (preferably 90-97 wt %) water, and 0.01-2 wt % (preferably 0.1-1 wt %) surfactant (preferably a surfactant as defined herein).
A79. The composition, method, product, or use of any of A1-A11, wherein the composition consists of or consists essentially of 0.001-5 wt % (preferably 0.01-1 wt %) metal complex (preferably a copper-drug complex, suitably copper aspirinate or copper salicylate), 0.1-20 wt % (preferably 1-10 wt %) of the one or more (preferably at least two, preferably two) glyceride(s), 85-99.9 wt % (preferably 90-97 wt %) water, and 0.01-2 wt % (preferably 0.1-1 wt %) surfactant (preferably a surfactant as defined herein).
A80. The composition, method, product, or use of any of A1-A11, wherein the composition comprises 0.001-5 wt % (preferably 0.01-1 wt %) metal complex (preferably a copper-drug complex, suitably copper aspirinate or copper salicylate), 0.1-20 wt % (preferably 1-10 wt %) of the one or more (preferably at least two, preferably two) glyceride(s), and 85-99.9 wt % (preferably 90-97 wt %) water.
A81. The composition, method, product, or use of any of A1-A11, wherein the composition comprises 0.001-5 wt % (preferably 0.01-1 wt %) metal complex (preferably a copper-drug complex, suitably copper aspirinate or copper salicylate), 0.1-20 wt % (preferably 1-10 wt %) of the one or more (preferably at least two, preferably two) glyceride(s), 85-99.9 wt % (preferably 90-97 wt %) water, and 0.01-2 wt % (preferably 0.1-1 wt %) surfactant (preferably a surfactant as defined herein).
A82. The composition, method, product, or use of any of A1-A11, wherein the composition consists of or consists essentially of 0.001-5 wt % (preferably 0.01-1 wt %) metal complex (preferably a copper-drug complex, suitably copper aspirinate or copper salicylate), 0.1-20 wt % (preferably 1-10 wt %) of the one or more (preferably at least two, preferably two) glyceride(s), 85-99.9 wt % (preferably 90-97 wt %) water, and 0.01-2 wt % (preferably 0.1-1 wt %) surfactant (preferably a surfactant as defined herein).
A83. The composition, method, product, or use of any of A1-A11, wherein the composition comprises 0.001-5 wt % (preferably 0.01-1 wt %) metal complex (preferably a copper-drug complex, suitably copper aspirinate or copper salicylate), 0.1-20 wt % (preferably 1-10 wt %) mixture of glyceryl monooleate and glyceryl monolinoleate in a respective weight ratio between 25:75 and 35:65, and 85-99.9 wt % (preferably 90-97 wt %) water.
A84. The composition, method, product, or use of any of A1-A11, wherein the composition comprises 0.001-5 wt % (preferably 0.01-1 wt %) metal complex (preferably a copper-drug complex, suitably copper aspirinate or copper salicylate), 0.1-20 wt % (preferably 1-10 wt %) mixture of glyceryl monooleate and glyceryl monolinoleate in a respective weight ratio between 25:75 and 35:65, 85-99.9 wt % (preferably 90-97 wt %) water, and 0.01-2 wt % (preferably 0.1-1 wt %) surfactant (preferably a surfactant as defined herein).
A85. The composition, method, product, or use of any of A1-A11, wherein the composition consists of or consists essentially of 0.001-5 wt % (preferably 0.01-1 wt %) metal complex (preferably a copper-drug complex, suitably copper aspirinate or copper salicylate), 0.1-20 wt % (preferably 1-10 wt %) mixture of glyceryl monooleate and glyceryl monolinoleate in a respective weight ratio between 25:75 and 35:65, 85-99.9 wt % (preferably 90-97 wt %) water, and 0.01-2 wt % (preferably 0.1-1 wt %) surfactant (preferably a surfactant as defined herein).
A86. The composition, method, product, or use of any of A1-A11, wherein the composition comprises, consists of, or consists essentially of: 0.0001-50 wt % metal complex (preferably a copper-drug complex, suitably copper aspirinate or copper salicylate), 0.1-99.999 wt % either or both glyceryl monooleate and/or glyceryl monolinoleate (preferably in a respective weight ratio between 25:75 and 35:65 when both), 0-99 wt % water, and 0-2 wt % surfactant (preferably a surfactant as defined herein).
A87. The composition, method, product, or use of any of A1-A87, wherein the “metal complex” is replaced by a metal compound.
A88. The composition, method, product, or use of A87, wherein the metal compound has one or more metal ion(s) (or atom(s)), suitably metal ion(s) (or atom(s)) as defined herein.
A89. The composition, method, product, or use of A87-A88, wherein the metal compound is free of a drug compound or drug ligand.
A90. The composition, method, product, or use of A87-A89, wherein the metal compound is an antimicrobial metal compound.
A91. The composition, method, product, or use of A87-A90, wherein the metal compound is an antifungal metal compound.
A92. The composition, method, product, or use of A87-A91, wherein the metal compound is an inorganic metal compound.
A93. The composition, method, product, or use of A87-A92, wherein the metal compound is selected from the group of a metal oxide, a metal halide (e.g. metal fluoride, metal chloride, metal bromide, metal iodide), a metal nitrate, a metal nitrite, a metal sulphate, a metal phosphate, and any combination thereof.
A94. The composition, method, product, or use of A93, wherein the metal compound is a metal oxide.
A95. The composition, method, product, or use of A87-A94, wherein the metal compound is a copper compound.
A96. The composition, method, product, or use of A95, wherein the copper compound is a copper (1) compound.
A97. The composition, method, product, or use of A95, wherein the copper compound is a copper (II) compound.
A98. The composition, method, product, or use of A95-A97, wherein the copper compound is an inorganic copper compound.
A99. The composition, method, product, or use of A95-A98, wherein the copper compound is an antimicrobial copper compound.
A100. The composition, method, product, or use of A95-A99, wherein the copper compound is an antifungal copper compound.
A101. The composition, method, product, or use of A95-A100, wherein the copper compound is selected from the group of a copper oxide, a copper halide (e.g. copper fluoride, copper chloride, copper bromide, copper iodide), a copper nitrate, a copper nitrite, a copper sulphate, a copper phosphate, and any combination thereof.

Specific embodiments A1-A101, and features thereof, may be further defined and/or sub-defined as set forth herein, in particular (and where compatible in the given context) as set forth in paragraphs B1-B427 below. In particular, any composition or product of any of specific embodiments A1-A101 may be a composition or product as defined in any of B2-B41. Any definition pertaining to a composition are equally applicable to methods, products, and uses of the invention, albeit, in preparative methods, ingredients of the compositions will generally be pertinent in the context of them being mixed together or otherwise combined or incorporated to provide relevant products of said methods (be them compositions or other products).

### Specific Embodiments and Quantities

The table below indicates suitable and increasingly-preferred (in descending from Range Number 1 to higher Range Numbers, e.g. 5) embodiments of concentrations (and concentration ranges, wt %) for each of the ingredients of a metal complex (MC) (which is preferably a copper-drug complex, suitably copper aspirinate or copper salicylate), glyceride(s) (G) (which preferably is or comprises either or both MO and/or DU), and solvent (which is preferably water). The table also indicates suitable and increasingly-preferred (in descending from Range Number 1 to higher Range Numbers, e.g. 5a/b) embodiments of weight ratios of metal complex (MC) (which is preferably a copper-drug complex, suitably copper aspirinate or copper salicylate) to glyceride(s) (G) (which preferably is or comprises either or both MO and/or DU).

| Ingredient / | | | | | | Some water, | | | | |
|---|---|---|---|---|---|---|---|---|---|---|
| Range | | Low water, no | | Low water + | | no | | Some water + | | |
| Number | General | encapsulation | | encapsulation | | encapsulation | | encapsulation | | Aqueous |
| Metal | 0.01-24.2 | 0.01 | wt % | 10.7-11.5 | wt % | 2.2-6.0 | wt % | 24.2 | wt % | 0.04-0.25 |
| Complex | wt % | | | | | | | | | wt % |
| (MC) | | | | | | | | | | |
| 1 | 0.0001-50 | 0.0001-1 | wt % | 1-30 | wt % | 0.01-20 | wt % | 1-50 | wt % | 0.001-5 |
| | wt % | | | | | | | | | wt % |
| 2 | 0.001-40 | 0.001-0.1 | wt % | 2-20 | wt % | 0.1-15 | wt % | 5-40 | wt % | 0.01-1 |
| | wt % | | | | | | | | | wt % |
| 3 | 0.005-30 | 0.005-0.05 | wt % | 5-15 | wt % | 1-10 | wt % | 10-30 | wt % | 0.02-0.5 |
| | wt % | | | | | | | | | wt % |
| 4 | | 0.01 | wt % | 10-12 | wt % | 2-7 | wt % | 20-30 | wt % | 0.02-0.06 |
| | | | | | | | | | | wt % |
| 5a | | | | | | 1-3 | wt % | 23-26 | wt % | 0.1-0.4 |
| | | | | | | | | | | wt % |
| 5b | | | | | | 4-8 | wt % | | | |
| Glyceride(s) | 4.96-99.99 | 99.99 | wt % | 88.5-89.3 | wt % | 56.4-58.7 | wt % | 45.5 | wt % | 4.96-4.97 |
| (G) | wt % | | | | | | | | | wt % |
| 1 | 0.1-99.999 | 10-99.9999 | wt % | 10-99 | wt % | 5-95 | wt % | 5-95 | wt % | 0.01-30 |
| | wt % | | | | | | | | | wt % |
| 2 | 1-99.99 | 50-99.999 | wt % | 50-95 | wt % | 30-90 | wt % | 20-80 | wt % | 0.1-20 |
| | wt % | | | | | | | | | wt % |
| 3 | 4-99.99 | 80-99.995 | wt % | 80-93 | wt % | 40-80 | wt % | 30-70 | wt % | 1-10 wt % |
| | wt % | | | | | | | | | |
| 4 | | 99.99 | wt % | 88-90 | wt % | 50-70 | wt % | 40-50 | wt % | 2-8 wt % |
| 5 | | | | | | 55-60 | wt % | 43-47 | wt % | 4-6 wt % |
| Solvent (e.g. | 0-94.51 | ~0 | wt % | ~0 | wt % | 37.6-39.1 | wt % | 30.3 | wt % | 94.32-94.51 |
| Water) | wt % | | | | | | | | | wt % |
| 1 | 0-99 wt % | ≤5 | wt % | ≤5 | wt % | 10-60 | wt % | 10-60 | wt % | 85-99.9 |
| | | | | | | | | | | wt % |
| 2 | 0.0001-98 | ≤2 | wt % | ≤2 | wt % | 15-60 | wt % | 10-50 | wt % | 45-99 wt % |
| | wt % | | | | | | | | | |
| 3 | 0.001-95 | ≤1 | wt % | ≤1 | wt % | 25-50 | wt % | 20-40 | wt % | 70-98 wt % |
| | wt % | | | | | | | | | |
| 4 | | ~0 | wt % | ~0 | wt % | 30-45 | wt % | 25-35 | wt % | 90-97 wt % |
| 5 | | | | | | 35-40 | wt % | 28-32 | wt % | 93-96 wt % |

| | | | | | | |
|---|---|---|---|---|---|---|
| MC:G (wt | 1-533 | 1 mg/g +/− | 120-130 | 38-106 | 533 | 8-50 |
| ratio) | mg/g +/− 40 | 0.5 | mg/g +/− 25 | mg/g +/− 15 | mg/g +/− 40 | mg/g +/− 5 |
| 1 | 0.01:1000- | 0.0001:1000- | 0.1:1000- | 0.1:1000- | 10:1000- | 0.1:1000- |
| | 900:1000 | 50:1000 | 500:1000 | 300:1000 | 900:1000 | 600:1000 |
| 2 | 0.1:1000- | 0.001:1000- | 10:1000- | 1:1000- | 50:1000- | 1:1000- |
| | 700:1000 | 10:1000 | 400:1000 | 200:1000 | 700:1000 | 200:1000 |
| 3 | 0.5:1000- | 0.1:1000- | 50:1000- | 10:1000- | 100:1000- | 5:1000- |
| | 600:1000 | 5:1000 | 200:1000 | 150:1000 | 600:1000 | 100:1000 |
| 4 | | 0.5:1000- | 90:1000- | 20:1000- | 200:1000- | 2:1000- |
| | | 1.5:1000 | 160:1000 | 130:1000 | 580:1000 | 20:1000 |
| 5a | | | | 10:1000- | 500:1000- | 30:1000- |
| | | | | 70:1000 | 550:1000 | 70:1000 |
| | | | | (20:1000- | | |
| | | | | 50:1000) | | |
| 5b | | | | 50:1000- | | |
| | | | | 150:1000 | | |
| | | | | (90:1000- | | |
| | | | | 120:1000) | | |

Suitable embodiments comprising or consisting of a combination of any, some, or all of a metal complex (MC) (which is preferably a copper-drug complex, suitably copper aspirinate or copper salicylate), glyceride(s) (G) (which preferably is or comprises either or both MO and/or DU), and solvent (which is preferably water), are herein disclosed by combining any, some, or all of the concentrations or weight ratios (in any of the Range Number slots) pertaining to the aforesaid ingredients.

As a person skilled in the art will readily appreciate, suitable and increasingly-preferred embodiments (when descending Range Numbers) comprising or consisting of a combination of any, some, or all of a metal complex (MC) (which is preferably a copper-drug complex, suitably copper aspirinate or copper salicylate), glyceride(s) (G) (which preferably is or comprises either or both MO and/or DU), and solvent (which is preferably water), are herein disclosed by combining relevant concentrations and/or weight ratios of the same Range Numbers in respect of the aforesaid ingredients.

### Specific Embodiments via Dependency

The following numbered paragraphs (B1-B427), with dependencies, describe further embodiments and features which may be combined with any embodiments and features disclosed in any other section herein, including the embodiments of A1-A101. A person skilled in the art will readily appreciate embodiments elucidated by the dependencies set forth below, including via direct and indirect dependencies.

B) Numbered Paragraphs with Dependencies
B1. A composition, either in general or suitably as defined anywhere herein, including as defined in any of paragraphs A1-A101 above (in particular where compatible in the given context).
B2. The composition of B1, wherein the composition is a topical composition (e.g. for use on a bodily surface, for example skin, eye(s), body cavity, and/or hair).
B3. The composition of B2, wherein the topical composition is a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.
B4. The composition of B3, wherein the topical composition is a cream, an ointment, a foam, or a perfume (e.g. perfume, aftershave, cologne, or eau de toilette).
B5. The composition of B4, wherein the topical composition is a cream.
B6. The composition of B4, wherein the topical composition is an ointment.
B7. The composition of B2, wherein the composition is an oral topical composition, for example, a mouthwash, a rinse, an oral spray, a suspension, and/or a dental gel, suitably which is intended to be taken by mouth but are not intended to be ingested.
B8. The composition of B1, wherein composition is an ingestible composition (e.g. orally-administrable composition).
B9. The composition of B8, wherein the ingestible composition is selected from a lozenge, a gel, a jelly, a pastille, a tablet, a capsule (e.g. a capsule containing the composition), a toffee, a nougat, a chewy candy, and/or a chewing gum.
B110. The composition of B9, wherein the ingestible composition is (or is otherwise incorporated within) a pastille, a gel, or a jelly.
B111. The composition of B8-B110, wherein the ingestible composition comprises a gel component, gelling component, or gellable component.
B112. The composition of B111, wherein the gel component, gelling component, or gellable component is animal- or plant-derived.
B113. The composition of B112, wherein the gel component, gelling component, or gellable component is plant-derived (suitably the composition or product into which said composition is incorporated is free of any animal-derived components/ingredients).
B14. The composition of B11-B13, wherein the gel component, gelling component, or gellable component comprises or consists of a polysaccharide or derivative thereof (e.g. x-carrageenan).
B15. The composition of B11-B14, wherein the gel component, gelling component, or gellable component comprises or consists of a gelatin, agar, and/or agarose.
B116. The composition of B8, wherein the ingestible composition is (or is otherwise incorporated within, for instance, by mixing the composition with a solvent such as water) a liquid, suitably a suspension, a dispersion, or a solution.
B117. The composition of B116, wherein the ingestible composition is a drink/beverage.
B18. The composition of B1-B17, wherein the composition is (or is otherwise incorporated within) a pharmaceutical composition.
B19. The composition of B18, wherein the pharmaceutical composition is for use as a medicament (or may otherwise be used in the manufacture of a medicament).
B20. The composition of B18, wherein the pharmaceutical composition is (or is otherwise incorporated within a product that is) an ayurvedic medicine.
B21. The composition of B18, wherein the pharmaceutical composition is for (or is for use in) treating: neurodegenerative disorders including Alzheimer's disease, Parkinson's disease, Huntington's disease, Prion disease, Spinal muscular atrophy, conditions caused by amyloids or amyloid plaques, inflammation, micriobial infections (e.g. composition as an antimicrobial composition), viral infections, fungal infections, depression, cancer(s) (particularly breast cancer, pancreatic cancer, colon cancer, multiple myeloma, myelogenous leukaemia, and colorectal cancer), anaphylaxis, arthritis, osteoarthritis, acne, and/or eczema.
B22. The composition of B1-B17, wherein the composition is (or is otherwise incorporated within) a nutraceutical composition.
B23. The composition of B1-B17, wherein the composition is (or is otherwise incorporated within) a food supplement.
B24. The composition of B1-B117, wherein the composition is (or is otherwise incorporated within) a food additive.
B25. The composition of B1-B117, wherein the composition is (or is otherwise incorporated within) a foodstuff.
B26. The composition of B1-B17, wherein the composition is a personal care composition.
B27. The composition of B26, wherein the personal care composition is selected from the group consisting of skin care compositions, skin creams, shaving compositions (e.g. shaving creams, gels, foams), moisturizers, lotions, body washes, body oils, and hair care compositions (e.g. shampoos and/or hair conditioners).
B28. The composition of B26-B27, wherein the composition is a “leave-on-type” composition (e.g. moisturizing lotions, serums, creams) or a “rinse-off-type” composition (e.g. body washes, shampoos, hair conditioners, shower gels, skin cleansers, cleansing milks, shaving compositions).
B29. The composition of B1-B28 (especially B26-B28), wherein the composition is in the form of (or is otherwise incorporated within) an emulsion, a lotion, a milk, a liquid, a solid, a cream, a gel, a mousse, an ointment, a paste, a serum, a stick, a spray, a tonic, an aerosol, a foam, and/or a pencil.
B30. The composition of B1-B29 (especially B26-B29), wherein the composition is in the form of (or is otherwise incorporated within) an implement (e.g. a powdered or unpowdered cosmetic applicator), a reusable or disposable wipe, a tissue, a towel, a diaper, a razor or other shaving device, a personal cleansing implement (e.g. a mesh shower sponge), a conventional sponge, a wash cloth, a swab, and/or a pen.
B31. The composition of B1-B30, wherein the composition is a cosmetic composition (or is a cosmetic product or is incorporated within a cosmetic product—e.g. a cosmetic product comprising the composition).
B32. The composition of B31, wherein the cosmetic composition is for controlling and/or reducing the formation of wrinkles and lines in the skin.
B33. The composition of B31, wherein the cosmetic composition is for providing glowing skin.
B34. The composition of B1-B17, wherein the composition is a wound care composition (or is a wound care product or is incorporated within a wound care product—e.g. a wound care product comprising the composition).
B35. The composition of B34, wherein the wound care (or wound healing) composition is a wound care hydrogel composition, and therefor suitable comprises a hydrogel or hydrogel-forming component or compound.
B36. The composition of B34-B35, wherein the wound care composition is incorporated within a wound care product—e.g. a wound care product comprising the composition.
B37. The composition of B36, wherein wound care product is selected from the group consisting of a wound dressing, a sutures, a staple, a gauze, a bandage, a plaster, a burn dressing, an artificial skin, a liposome or micell formulation, a microcapsule, an aqueous vehicle for soaking gauze dressings.
B38. The composition of B34-B37, wherein the composition is for (or for use in) healing wounds, for example, healing cuts and scrapes, burns, ulcers, bed sores, fissures, haemorrhoids, and/or post-surgical wounds.
B39. The composition of B1-B17, wherein the composition is (or is otherwise incorporated within) a textile.
B40. The composition of B1-B39, wherein the composition is an oil-in-water dispersion.
B41. The composition of B1-B39, wherein the composition is an water-in-oil dispersion.
   Metal Complex B42. The composition of B1-B41, comprising a metal complex (or metal-ligand complex), most preferably a copper-drug complex.
B43. The composition of B42, wherein the metal complex comprises one or more metal atoms and/or ions (most preferably ions) and one or more ligands.
B44. The composition of B43, wherein one or more of the one or more ligands is a drug (i.e. drug compound or drug ligand).
B45. The composition of B44, wherein the metal complex is a metal-drug complex (i.e. a or a plurality of metal(s) datively bonded to a drug molecule/compound, suitably via one or more, preferably two or more, lone pair(s) of electrons borne by the drug).
B46. The composition of B45, wherein the drug represents a multidentate ligand to the metal atom(s) and/or ion(s).
B47. The composition of B42-B46, wherein the metal complex is an aqueous-insoluble metal complex, most suitably an aqueous-insoluble metal-drug complex.
B48. The composition of B47, wherein the metal complex is practically insoluble (10,000 parts solvent required per part of solute), very slightly soluble (1,000-10,000 parts solvent required per part of solute), or slightly soluble (100-1,000 parts solvent required per part of solute), in accordance with solubility definitions of the US Pharmacopeia (USP).
B49. The composition of B48, wherein the metal complex is either practically insoluble (10,000 parts solvent required per part of solute) or very slightly soluble (1,000-10,000 parts solvent required per part of solute), in accordance with solubility definitions of the US Pharmacopeia (USP).
B50. The composition of B49, wherein the metal complex is practically insoluble (10,000 parts solvent required per part of solute) in accordance with solubility definitions of the US Pharmacopeia (USP).
B51. The composition of B42-B50, wherein the metal complex is a transition metal complex.
B52. The composition of B51, wherein the transition metal complex is an aqueous-insoluble transition metal-drug complex.
B53. The composition of B42-B52, wherein the metal complex is monometallic, suitably in that the complex comprises one metal atom or metal ion (most preferably metal ion) within said complex.
B54. The composition of B42-B52, wherein the metal complex is polymetallic (e.g. bimetallic) (i.e. a polymetallic complex) in that the complex comprises one or a plurality (e.g. two) metal atom(s) and/or ion(s) (most preferably ion(s)) within said complex.
B55. The composition of B54, wherein the polymetallic complex is homometallic (i.e. where all of the plurality of metal atom(s) and/or ion(s) are derived from the same metal element).
B56. The composition of B54, wherein the polymetallic complex is heterometallic (i.e. where at least two of the metal atoms and/or ions are derived from different metal elements).
B57. The composition of B54-B56, wherein the polymetallic complex is homovalent (i.e. where all of the plurality of metal atom(s) and/or ion(s) are derived from the same or different metal elements but which all have the same valency or have the same numerical oxidation state) B58. The composition of B54-B56, wherein the polymetallic complex is heterovalent (i.e. where at least two of the metal atoms and/or ions, which may be derived from the same or different metal elements, have a different valency or oxidation state).
B59. The composition of B53-B58, wherein the metal complex is monometallic, polymetallic (preferably bimetallic), or a mixture thereof.
B60. The composition of B59, wherein the metal complex is homometallic.
B61. The composition of B60, wherein the metal complex is homovalent.
B62. The composition of B42-B61, wherein the metal complex is a copper complex (or copper-ligand complex) B63. The composition of B62, wherein the copper complex comprises one or more copper atoms and/or copper ions (most preferably copper ions, most preferably copper 2+ ions) and one or more ligands. Most preferably, the copper complex is a copper(II) complex (i.e. with copper in the 2+ oxidation state, or having a valency of 2).
B64. The composition of B63, wherein one or more of the one or more ligands is a drug (i.e. drug compound/molecule or drug ligand).
B65. The composition of B64, wherein the copper complex is a copper-drug complex (i.e. a or a plurality of copper(s) datively bonded to a drug molecule/compound, suitably via one or more, preferably two or more, lone pair(s) of electrons borne by the drug).
B66. The composition of B65, wherein the drug represents a multidentate ligand to the copper atom(s) and/or copper ion(s) (most preferably copper(II) ions).
B67. The composition of B64 or B65, wherein the copper complex is an aqueous-insoluble copper-drug complex, most suitably an aqueous-insoluble copper(II)-drug complex (where insoluble is suitably as defined herein, though most preferably means practically insoluble according to USP).
B68. The composition of B62-B67, wherein the copper complex is monometallic, suitably in that the complex comprises one copper atom or copper ion (preferably copper ion, most preferably a copper(II) ion) within said complex.
B69. The composition of B62-B67, wherein the copper complex is polymetallic (e.g. bimetallic) (i.e. a polymetallic complex) in that the complex comprises one or a plurality (e.g. two) copper atom(s) and/or copper ion(s) (most preferably ion(s), most preferably copper(II) ion(s)) within said complex.
B70. The composition of B69, wherein the polymetallic complex is homometallic (i.e. where all of the plurality of metal atom(s) and/or ion(s) are derived from the element copper) B71. The composition of B69, wherein the polymetallic complex is heterometallic (i.e. where at least one of the metal atoms and/or ions are derived from the element copper, and at least one other of the metal atoms and/or ions are derived from different metal elements).
B72. The composition of B69-B71, wherein the polymetallic complex is homovalent (i.e. where all of the plurality of metal atom(s) and/or ion(s), whether or not all or some are derived from the element copper, which all have the same valency or have the same numerical oxidation state).
B73. The composition of B69-B71, wherein the polymetallic complex is heterovalent (i.e. where at least two of the metal atoms and/or ions, whether or not all or some are derived from the element copper, have a different valency or oxidation state).
B74. The composition of B62-B73, wherein the copper complex is monometallic, polymetallic (preferably bimetallic), or a mixture thereof.
B75. The composition of B74, wherein the copper complex is homometallic (i.e. all metal atoms and/or ions are derived from copper).
B76. The composition of B75, wherein the copper complex is homovalent (preferably all copper atoms and/or ions have the same valency or oxidation state).
B77. The composition of B42-B76, wherein the metal complex (preferably copper complex, most preferably copper(II) complex) is a metal-drug complex (preferably copper-drug complex, most preferably copper(II)-drug complex, most preferably an aqueous-insoluble copper(II)-drug complex) wherein one or more of the one or more ligands of the complex is a drug (i.e. drug compound/molecule or drug ligand) or ionised form of said drug.
B78. The composition of B77, wherein the metal complex is a metal-drug complex comprising one or more copper ions (preferably copper(II) ions) and one or more drug molecule(s) (or ionised form(s) thereof).
B79. The composition of B77-B78, wherein the drug is a drug compound/molecule or an ionised form thereof (e.g. carboxylate corresponding to a carboxylic acid moiety), which is datively bonded (suitably in a monodentate or multidentate fashion, suitable via one or more lone pair(s) of electrons borne by the drug) to one or more of the metal atoms and/or metal ions of the metal complex.
B80. The composition of B79, wherein the drug is asprin (or aspirinate).
B81. The composition of B79, wherein the drug is salicylic acid (salicylate) (or a derivative thereof).
B82. The composition of B77-B81, wherein the metal-drug complex (e.g. copper aspirinate) comprises other ligands (e.g. solvates or other appropriate ligands) in addition to one or more drug molecule(s).
B83. The composition of B77-B82, wherein the metal complex is copper(II) salicylate (or any suitable complex or salt thereof).
B84. The composition of B77-B82, wherein the metal complex is copper(II) aspirinate (or any suitable complex or salt thereof).
B85. The composition of B42-B84, wherein the metal complex is substantially pure (e.g. greater than or equal to 95 wt % pure, suitably greater than or equal to 98 wt % pure, preferably greater than or equal to 99 wt % pure).

Metal Ion(s) (or metal atom(s)) B86. The composition of B42-B85, wherein the metal complex comprises one or more metal atoms and/or ions (most preferably ions), suitably along with one or more ligands (suitably as defined herein).
B87. The composition of B86, wherein the metal complex comprises one or more metal ions.
B88. The composition of B86, wherein the metal complex comprises one or more transition-metal ions.
B89. The composition of B86, wherein the metal complex comprises one or more copper ions.
B90. The composition of B86, wherein the metal complex comprises one or more copper(II) ions (i.e. Cu²⁺ ions).
B91. The composition of B86-B90, wherein the metal complex is monometallic and/or polymetallic (e.g. bimetallic).
B92. The composition of B91, wherein where the metal complex is polymetallic (especially when bimetallic) or comprises polymetallic complex, the polymetallic complex is homometallic in that all of the plurality of ions are derived from the same metal element (preferably copper), and homovalent. in that all of the plurality of metal ions (preferably copper ions) have the same valency (e.g. Cu²⁺).
B93. The composition of B91-B92, wherein the metal complex is a mixture of monometallic and polymetallic (preferably bimetallic) forms of said metal complex.
B94. The composition of B86-B93, wherein the metal ion(s) (or, where relevant, metal atom(s)) is/are co-ordinated to one or more drug molecule(s) and optionally to one or more additional ligands.

### Drug Molecule

B95. The composition of B42-B94, wherein the metal complex is a metal-drug complex (suitably copper-drug complex, suitably a copper(II)-drug complex) comprising a drug molecule (which includes any ionised forms thereof, e.g. carboxylate if the drug molecular contains a carboxylic acid moiety) as a ligand (though optionally a counterion instead or in addition) within the metal-drug complex.
B96. The composition of B95, wherein the metal-drug complex suitably comprises one or more (a plurality of) drug molecules (preferably as ligands).
B97. The composition of B96, wherein the drug molecule is any drug molecule capable of co-ordinating to (or with) the metal atom(s) and/or metal ion(s) within the metal-drug complex. NB. The drug molecule (which includes any ionised form thereof) may be abbreviated simply to “drug”.
B98. The composition of B97, wherein the drug molecule comprises one or more moieties capable of donating one or more lone pair(s) of electrons.
B99. The composition of B98, wherein the drug molecule is datively bonded to one or more metal atoms and/or metal ions of the metal-drug complex.
B100. The composition of B99, wherein the drug molecule co-ordinates within the complex in a multidentate fashion, be it with a single metal atom or metal ion or with two or more metal atoms or metal ions.
B101. The composition of B95-B100, wherein the drug molecule is a carboxylic acid or (given that the definition of a drug molecule includes any ionised forms thereof) a carboxylate.
B102. The composition of B101, wherein the carboxylic acid (or carboxylate) co-ordinates in a bidentate fashion within the complex, be it with a single metal atom or metal ion or with two or more metal atoms or metal ions. B103. The composition of B101-B102, wherein the drug molecule is defined by Formula I:

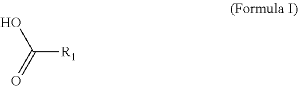

wherein R₁ is an optionally substituted organic (i.e. carbon-containing) group suitably which together with the carboxylic acid moiety completes the drug molecule.
B104. The composition of B103, wherein R₁ is an optionally substituted hydrocarbyl group (suitably optionally substituted by one or more moieties independently selected from Rₓ), wherein adjacent carbon atoms within the hydrocarbyl group are optionally separated by a linker -L-, for instance by the insertion thereinto of a heteroatom (e.g. 0, S) or heteroatom-containing moiety (e.g. O, S, SO, SO₂, N(Rₐ), CO, C(O)O, OC(O), OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group).
B105. The composition of B103, wherein R₁ is or comprises one or more optionally substituted ring systems (suitably optionally substituted by one or more moieties independently selected from Rₓ), fused or otherwise, wherein adjacent carbon atoms within the hydrocarbyl group are optionally separated by a linker -L-, for instance by the insertion thereinto of a heteroatom (e.g. 0, S) or heteroatom-containing moiety (e.g. O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group).
B106. The composition of B103, wherein R₁ is or comprises an optionally substituted moiety (suitably optionally substituted by one or more moieties independently selected from Rₓ) selected from the group consisting of aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl, heterocyclyl-(1-6C)alkyl, and any combination thereof.
B107. The composition of B103, wherein R₁ is or comprises an optionally substituted moiety (suitably optionally substituted by one or more moieties independently selected from Rₓ) selected from of aryl, aryl-(1-6C)alkyl, heteroaryl, heteroaryl-(1-6C)alkyl, and any combination thereof.
B108. The composition of B103, wherein R₁ is or comprises an optionally substituted (suitably optionally substituted by one or more moieties independently selected from Rₓ) aryl.
B109. The composition of B103, wherein R₁ is or comprises an optionally substituted (suitably optionally substituted by one or more moieties independently selected from Rₓ) phenyl.
B110. The composition of B104-B109, wherein Rₓ is selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

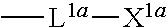

wherein:
L^{1a} is absent or is selected from O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl; wherein any Rₓ group is optionally further substituted with one or more Rₓ groups as defined herein.
B111. The composition of B110, wherein Rₓ is selected from hydrogen, halogeno, trifluoromethyl, cyano, isocyano, nitro, hydroxy, mercapto, amino, formyl, carboxy, carbamoyl, ureido, (1-8C)alkyl, (1-8C)hydroxyalkyl, (1-6C)alkoxy, (1-6C)alkylamino, (1-6C)dialkylamino, (2-6C)alkanoyl, (2-6C)alkanoyloxy, or from a group of the formula:

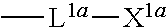

wherein:
   L^{1a} is absent or is selected from O, S, SO, SO₂, N(R_{B}), CO, C(O)O, CH(OR_{B}), CON(R_{B}), N(R_{B})CO, N(R_{B})CON(R_{B}), SO₂N(R_{B}), N(R_{B})SO₂, OC(R_{B})₂, SC(R_{B})₂ and N(R_{B})C(R_{B})₂, wherein where Rₐ and R_{b}, which may be the same or different, are each independently selected from hydrogen or (1-8C)alkyl; and X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl; wherein any Rₓ group is optionally further substituted with one or more Rₓ groups as defined herein.
B112. The composition of B111, wherein Rₓ is selected from halogeno, trifluoromethyl, cyano, hydroxy, amino, carboxy, (1-8C)alkyl, (1-8C)hydroxyalkyl, (1-6C)alkoxy, (2-6C)alkanoyl, and (2-6C)alkanoyloxy.
B113. The composition of B112, wherein Rₓ is selected from hydroxy, (1-6C)alkoxy, and (2-6C)alkanoyloxy.
B114. The composition of B113, wherein Rₓ is selected from hydroxy and (2-6C)alkanoyloxy.
B115. The composition of B114, wherein Rₓ is selected from hydroxy (—OH) and acetoxy (—OAc or —OC(O)CH₃).
B116. The composition of B104-B105 or any preceding paragraph dependent thereon, wherein the linker, -L-, is suitably selected from O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group defined in any of B110-B115.
B117. The composition of B103, wherein the drug molecule is defined by Formula II:

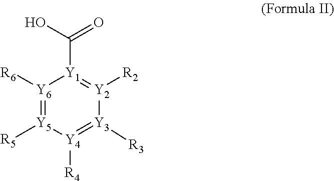

wherein Y₁ is C, and each of Y₂, Y₃, Y₄, Y₅, and Y₆ are independently selected from C, N, and O (where each of R₂, R₃, R₄, R₅, and R₆ are correspondingly compatible, and may suitably be absent where their corresponding Y atom is O or N);
wherein each of R₂, R₃, R₄, R₅, and R₆ are independently either absent (e.g. if the corresponding Y₁-Y₆ to which they are connected is either O or N), hydrogen, or an Rₓ as defined in any of B110-B115; or any ionised form thereof.
B118. The composition of B103, wherein the drug molecule is defined by Formula III:

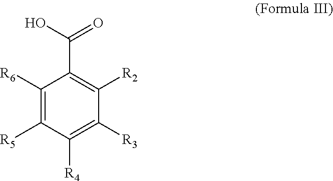

wherein each of R₂, R₃, R₄, R₅, and R₆ are independently either hydrogen or an Rₓ as defined in any of B110-B115 (though more suitably where Rₓ is selected from hydroxy, (1-6C)alkoxy, and (2-6C)alkanoyloxy, and most suitably wherein Rₓ is selected from hydroxy and acetoxy); or any ionised form thereof.
B119. The composition of B103, wherein the drug molecule is defined by Formula IV:

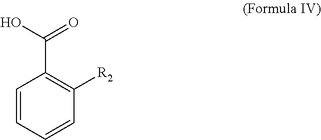

wherein R₂ is either hydrogen or an Rₓ as defined in any of B110-B115 (though more suitably where Rₓ is selected from hydroxy, (1-6C)alkoxy, and (2-6C)alkanoyloxy, and most suitably wherein Rₓ is selected from hydroxy and acetoxy); or any ionised form thereof.
B120. The composition of B103, wherein the drug molecule is defined by Formula V:

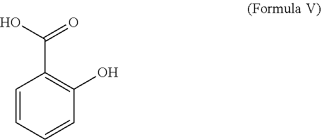

or any ionised form thereof.
B121. The composition of B103, wherein the drug molecule is defined by Formula VI:

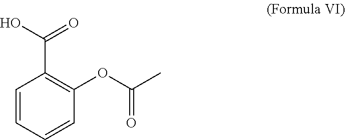

or any ionised form thereof.
B122. The composition of B95, wherein the drug molecule is salicylic acid (or any ionised form thereof).
B123. The composition of B95, wherein the drug molecule is aspirin (acetylsalicylic acid) (or any ionised form thereof).

### Metal Compound

B124. The composition of B1-B123, comprising a metal compound (suitably the term “metal compound” may replace “metal complex” in any of B1-B123, and suitably the “metal compound” is instead of said “metal complex” rather than in addition to).
B125. The composition of B124, wherein the “metal complex” is replaced by a metal compound.
B126. The composition of B124-B125, wherein the metal compound has one or more metal ion(s) (or atom(s)), suitably metal ion(s) (or atom(s)) as defined herein (e.g. B86-B94).
B127. The composition of B124-B126, wherein the metal compound is free of a drug compound or drug ligand.
B128. The composition of B124-B127, wherein the metal compound is an antimicrobial metal compound.
B129. The composition of B124-B128, wherein the metal compound is an antifungal metal compound.
B130. The composition of B124-B129, wherein the metal compound is an inorganic metal compound.
B131. The composition of B124-B130, wherein the metal compound is selected from the group of a metal oxide, a metal halide (e.g. metal fluoride, metal chloride, metal bromide, metal iodide), a metal nitrate, a metal nitrite, a metal sulphate, a metal phosphate, and any combination thereof.
B132. The composition of B131, wherein the metal compound is a metal oxide.
B133. The composition of B124-B132, wherein the metal compound is a copper compound.
B134. The composition of B133, wherein the copper compound is a copper (1) compound.
B135. The composition of B133, wherein the copper compound is a copper (II) compound.
B136. The composition of B133-B135, wherein the copper compound is an inorganic copper compound.
B137. The composition of B133-B136, wherein the copper compound is an antimicrobial copper compound.
B138. The composition of B133-B137, wherein the copper compound is an antifungal copper compound.
B139. The composition of B138, wherein the copper compound is selected from the group of a copper oxide, a copper halide (e.g. copper fluoride, copper chloride, copper bromide, copper iodide), a copper nitrate, a copper nitrite, a copper sulphate, a copper phosphate, and any combination thereof.
B140. The composition of B139, wherein the copper compound is a copper oxide.

### Glycerides

B141. The composition of B1-B140, wherein the composition comprises one or more glycerides.
B142. The composition of B141, wherein the composition comprises a glyceride.
B143. The composition of B141, wherein the composition comprises a mono- and/or di-glyceride.
B144. The composition of B141, wherein the composition comprises a monoglyceride.
B145. The composition of B141, wherein the composition comprises two or more glycerides.
B146. The composition of B141, wherein the composition comprises at most two glycerides.
B147. The composition of B141, wherein the composition comprises a first glyceride and a second glyceride.
B148. The composition of B141, wherein the composition comprises a first glyceride and a second glyceride in a weight ratio between 1:99 and 99:1.
B149. The composition of B141, wherein the composition comprises a first glyceride and a second glyceride in a weight ratio between 1:99 and 90:10.
B150. The composition of B141, wherein the composition comprises a first glyceride and a second glyceride in a weight ratio between 10:90 and 90:10.
B151. The composition of B141, wherein the composition comprises a first glyceride and a second glyceride in a weight ratio between 10:90 and 50:50.
B152. The composition of B141, wherein the composition comprises a first glyceride and a second glyceride in a weight ratio between 20:80 and 40:60.
B153. The composition of B141, wherein the composition comprises a first glyceride and a second glyceride in a weight ratio between 25:75 and 35:65.
B154. The composition of B141, wherein the composition comprises a first glyceride and a second glyceride in a weight ratio between 27:73 and 30:70.
B155. The composition of B141, wherein the composition comprises one and at most one glycerides.
B156. The composition of B141, wherein the composition comprises two and at most two glycerides.
B157. The composition of B141-B156, wherein the glyceride(s) is/are mono- and/or di-glyceride(s).
B158. The composition of B141-B156, wherein the glyceride(s) is/are mono-glyceride(s).
B159. The composition of B141-B158, wherein at least one of the glyceride(s) has a melting point of 25-55° C.
B160. The composition of B141-B158, wherein at least two (preferably the two which are mentioned/defined) of the glyceride(s) have a melting point of 25-55° C.
B161. The composition of B141-B158, wherein each defined glyceride(s) each has a melting point of 25-55° C.
B162. The composition of B141-B158, wherein at least one of the glyceride(s) has a melting point of 30-45° C.
B163. The composition of B141-B158, wherein at least two (preferably the two which are mentioned/defined) of the glyceride(s) have a melting point of 30-45° C.
B164. The composition of B141-B158, wherein each defined glyceride(s) each has a melting point of 30-45° C.
B165. The composition of B141-B158, wherein at least one of the glyceride(s) has a melting point of 34-41° C.
B166. The composition of B141-B158, wherein at least two (preferably the two which are mentioned/defined) of the glyceride(s) have a melting point of 34-41° C.
B167. The composition of B141-B158, wherein each defined glyceride(s) each has a melting point of 34-41° C.

Moieties on Glycerides B168. The composition of B141-B167, wherein the, each, or at least one of the, glyceride(s) comprises one or more unsaturated moieties.
B169. The composition of B168, wherein the, each, or at least one of the, glyceride(s) comprises between one and two unsaturated moieties.
B170. The composition of B168-B169, wherein the, each, or at least one of the, glyceride(s) comprises one (i.e. a single, one and only one) unsaturated moiety.
B171. The composition of B168-B170, wherein the, each, or at least one of the, glyceride(s) comprises two (i.e. two and only two) unsaturated moieties.
B172. The composition of B168-B171, wherein the composition comprises two glycerides, a first glyceride with one (i.e. one and only one) unsaturated moiety and a second glyceride with two (i.e. two and only two) unsaturated moieties. Suitably the respective first and second glyceride may be present in one of the aforementioned weight ratios.
B173. The composition of B168-B172, wherein the, each, or at least one of the, unsaturated moiety(ies) is/are an alkene moiety(ies).
B174. The composition of B173, wherein the, each, or at least one of the, alkene moiety(ies) is/are cis-alkene moiety(ies).
B175. The composition of B141-B174, wherein the, each, or at least one of the, glyceride(s) comprises one, two, or three fatty acid(s) (suitably respectively comprises one or two fatty acids in the case of mono- and/or diglyceride, and suitably respectively comprises only one fatty acid in the case of mono-glycerides) condensed with glycerol.
B176. The composition of B175, wherein the, each, or at least one of the, fatty acid(s) is/are aliphatic fatty acid(s).
B177. The composition of B175, wherein the, each, or at least one of the, fatty acid(s) is/are linear (i.e. unbranched) fatty acid(s).
B178. The composition of B175, wherein the, each, or at least one of the, fatty acid(s) is/are C₆-C₃₀ fatty acid(s) (i.e. having a carbon chain that is 6-30 carbons in length, including the carboxylate carbon).
B179. The composition of B175-B178, wherein the, each, or at least one of the, fatty acid(s) is/are C₈-C₂₀ fatty acid(s).
B180. The composition of B175-B179, wherein the, each, or at least one of the, fatty acid(s) is/are C₁₈ fatty acid(s).
B181. The composition of B175-B180, wherein the, each, or at least one of the, fatty acid(s) comprises one or more unsaturated moieties.
B182. The composition of B181, wherein the, each, or at least one of the, fatty acid(s) comprises between one and two unsaturated moieties.
B183. The composition of B181-B182, wherein the, each, or at least one of the, fatty acid(s) comprises one (i.e. a single, one and only one) unsaturated moiety.
B184. The composition of B181-B183, wherein the, each, or at least one of the, fatty acid(s) comprises two (i.e. two and only two) unsaturated moieties.
B185. The composition of B181-B184, wherein the composition comprises a first glyceride comprising a fatty acid (suitably only a single fatty acid, as per a mono-glyceride) with one or more unsaturated moiety(ies), and a second glyceride comprising a fatty acid (suitably only a single fatty acid, as per a mono-glyceride) with one or more unsaturated moiety(ies).
B186. The composition of B181-B185, wherein the composition comprises a first glyceride comprising a fatty acid (suitably only a single fatty acid, as per a mono-glyceride) with one (i.e. one and only one) unsaturated moiety, and a second glyceride comprising a fatty acid (suitably only a single fatty acid, as per a mono-glyceride) with two (i.e. two and only two) unsaturated moieties.
B187. The composition of B181-B186, wherein the, each, or at least one of the, unsaturated moiety(ies) is/are an alkene moiety(ies).
B188. The composition of B187, wherein the, each, or at least one of the, alkene moiety(ies) is/are cis-alkene moiety(ies).
B189. The composition of B181-B188, wherein the, each, or at least one of the, fatty acid(s) is oleic acid.
B190. The composition of B181-B189, wherein the, each, or at least one of the, fatty acid(s) is linoleic acid.
B191. The composition of B1-B190, wherein the composition comprises glyceryl monooleate (monoolein).
B192. The composition of B1-B191, wherein the composition comprises glyceryl monolinoleate (monolinolein).
B193. The composition of B1-B192, wherein the composition comprises glyceryl monooleate (monoolein) and glyceryl monolinoleate (monolinolein).
B194. The composition of B193, wherein the composition comprises glyceryl monooleate and glyceryl monolinoleate in a weight ratio between 1:99 and 90:10.
B195. The composition of B193, wherein the composition comprises glyceryl monooleate and glyceryl monolinoleate in a weight ratio between 10:90 and 50:50.
B196. The composition of B193, wherein the composition comprises glyceryl monooleate and glyceryl monolinoleate in a weight ratio between 20:80 and 40:60.
B197. The composition of B193, wherein the composition comprises glyceryl monooleate and glyceryl monolinoleate in a weight ratio between 25:75 and 35:65.
B198. The composition of B193, wherein the composition comprises glyceryl monooleate and glyceryl monolinoleate in a weight ratio between 27:73 and 30:70.

### Solvent/Water

B199. The composition of B1-B198, wherein the composition comprises a solvent, preferably water.
B200. The composition of B199, wherein the solvent is a polar solvent.
B201. The composition of B200, wherein the polar solvent is a protic solvent.
B202. The composition of B201, wherein the protic solvent is water.
   Additional Ingredients (or ingredients which are limited in terms of their presence)
B203. The composition of B1-B202, wherein the composition comprises one or more additional ingredients (or the composition is otherwise characterised by a maximum amount/concentration/ratio of said one or more additional ingredients, in which case the composition may be free of said additional ingredient(s) or comprise a stipulated maximum quantity thereof).
B204. The composition of B203, wherein the one or more additional ingredients comprise one or more ingredients selected from the group consisting of additional active(s), additional lipid(s) (e.g. fatty acid(s)), natural oil(s), bile salt(s) (and/or bile acid(s)), hydrophilic molecule(s), hydrophobic molecule(s), amphiphilic molecule(s), surfactant(s), or any combination thereof.
B205. The composition of B1-B202, wherein, save for active(s) or the metal complex, the composition consists of only GRAS grade components.

### Additional Lipids (or Glycerides)

B206. The composition of B203-B205, wherein the one or more additional ingredients comprise one or more additional lipids.
B207. The composition of B206, wherein the one or more additional lipids comprises one or more glyceride(s) other than those stipulated (e.g. those stipulated in B141-B198), suitably where said one or more glyceride(s) other than those stipulated may be referred to as “unstipulated” glyceride(s).
B208. The composition of B207, wherein the weight ratio of unstipulated glyceride(s) (e.g. those other than the glyceride(s) stipulated in B141-B198) to stipulated glyceride(s) (e.g. those stipulated in B141-B198) is between 0:100 to 50:50 (i.e. the weight ratio is less than or equal to 50:50).
B209. The composition of B207, wherein the weight ratio of unstipulated glyceride(s) (e.g. those other than the glyceride(s) stipulated in B141-B198) to stipulated glyceride(s) (e.g. those stipulated in B141-B198) is between 0:100 to 30:70 (i.e. the weight ratio is less than or equal to 30:70).
B210. The composition of B207, wherein the weight ratio of unstipulated glyceride(s) (e.g. those other than the glyceride(s) stipulated in B141-B198) to stipulated glyceride(s) (e.g. those stipulated in B141-B198) is between 0:100 to 20:80 (i.e. the weight ratio is less than or equal to 20:80).
B211. The composition of B207, wherein the weight ratio of unstipulated glyceride(s) (e.g. those other than the glyceride(s) stipulated in B141-B198) to stipulated glyceride(s) (e.g. those stipulated in B141-B198) is between 0:100 to 15:85 (i.e. the weight ratio is less than or equal to 15:85).
B212. The composition of B207, wherein the weight ratio of unstipulated glyceride(s) (e.g. those other than the glyceride(s) stipulated in B141-B198) to stipulated glyceride(s) (e.g. those stipulated in B141-B198) is between 0:100 to 10:90 (i.e. the weight ratio is less than or equal to 10:90).
B213. The composition of B207, wherein the weight ratio of unstipulated glyceride(s) (e.g. those other than the glyceride(s) stipulated in B141-B198) to stipulated glyceride(s) (e.g. those stipulated in B141-B198) is between 0:100 to 5:95 (i.e. the weight ratio is less than or equal to 5:95).
B214. The composition of B206, wherein the one or more additional lipids comprises one or more lipid(s) other than stipulated glyceride(s) (e.g. those stipulated in B141-B198), suitably where said one or more lipid(s) other than those stipulated may be referred to as “unstipulated” lipid(s).
B215. The composition of B214, wherein the weight ratio of unstipulated lipid(s) (e.g. lipid(s) other than the glyceride(s) stipulated in B141-B198) to stipulated glyceride(s) (e.g. those stipulated in B141-B198) is between 0:100 to 50:50 (i.e. the weight ratio is less than or equal to 50:50).
B216. The composition of B214, wherein the weight ratio of unstipulated lipid(s) (e.g. lipid(s) other than the glyceride(s) stipulated in B141-B198) to stipulated glyceride(s) (e.g. those stipulated in B141-B198) is between 0:100 to 30:70 (i.e. the weight ratio is less than or equal to 30:70).
B217. The composition of B214, wherein the weight ratio of unstipulated lipid(s) (e.g. lipid(s) those other than the glyceride(s) stipulated in B141-B198) to stipulated glyceride(s) (e.g. those stipulated in B141-B198) is between 0:100 to 20:80 (i.e. the weight ratio is less than or equal to 20:80).
B218. The composition of B214, wherein the weight ratio of unstipulated lipid(s) (e.g. lipid(s) those other than the glyceride(s) stipulated in B141-B198) to stipulated glyceride(s) (e.g. those stipulated in B141-B198) is between 0:100 to 15:85 (i.e. the weight ratio is less than or equal to 15:85).
B219. The composition of B214, wherein the weight ratio of unstipulated lipid(s) (e.g. lipid(s) other than the glyceride(s) stipulated in B141-B198) to stipulated glyceride(s) (e.g. those stipulated in B141-B198) is between 0:100 to 10:90 (i.e. the weight ratio is less than or equal to 10:90).
B220. The composition of B214, wherein the weight ratio of unstipulated lipid(s) (e.g. lipid(s) other than the glyceride(s) stipulated in B141-B198) to stipulated glyceride(s) (e.g. those stipulated in B141-B198) is between 0:100 to 5:95 (i.e. the weight ratio is less than or equal to 5:95).

### Additional Active

B221. The composition of B203-B205, wherein the one or more additional ingredients comprise an additional active ingredient (e.g. in addition to the metal complex).
B222. The composition of B221, wherein the additional active ingredient is other than a (different) metal complex (optionally with a different metal but the same drug, the same metal but a different drug, or a different drug and different metal).

### Additional Surfactant

B223. The composition of B203-B222, wherein the one or more additional ingredients comprise a surfactant. Suitably, in the context of the term “surfactant”, this excludes glyceride(s) or at least excludes any glyceride(s) defined/stipulated.
B224. The composition of B223, wherein the surfactant is a non-ionic surfactant.
B225. The composition of B223, wherein the surfactant is a non-ionic surfactant selected from the group consisting of: a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, polyglycerol esters, sucrose esters, saponins (e.g. saponin), and any combination thereof.
B226. The composition of B223, wherein the surfactant is a non-ionic surfactant selected from the group consisting of: a fatty alcohol, a fatty alcohol ether, a fatty acid ester, a fatty acid amide, alpha-tocopherol, polyglycerol esters, sucrose esters, saponins (e.g. saponin), and any combination thereof.
B227. The composition of B223, wherein the surfactant selected from the group consisting of: polyglycerol esters, sucrose esters, saponins (e.g. saponin), and any combination thereof.
B228. The composition of B223, wherein the surfactant is a saponin (e.g. saponin).
B229. The composition of B223, wherein the surfactant is a poloxamer.
B230. The composition of B229, wherein the surfactant is poloxamer 407.
B231. The composition of B223-B228, on the proviso that the surfactant is not a PEG-based surfactant (e.g. the surfactant does not comprise polyethylene glycol polymers or copolymeric blocks of polyethylene glycol).
B232. The composition of B223-B228, on the proviso that the surfactant is not a poloxamer.
B233. The composition of B223-B228, on the proviso that the surfactant is not poloxamer 407.
B234. The composition of B223-B228, on the proviso that the surfactant is not polysorbate 20.
B235. The composition of B223-B228, on the proviso that the surfactant is not PEG-40 castor oil (e.g. not Killiphor RH 40).
B236. The composition of B223-B235, wherein the surfactant is present (suitably where the composition comprises greater than or equal to 85 wt % water) at a concentration of 0.001-10 wt % of the overall composition.
B237. The composition of B223-B235, wherein the surfactant is present (suitably where the composition comprises greater than or equal to 85 wt % water) at a concentration of 0.01-2 wt % of the overall composition.
B238. The composition of B223-B235, wherein the surfactant is present (suitably where the composition comprises greater than or equal to 85 wt % water) at a concentration of 0.1-1.5 wt % of the overall composition.
B239. The composition of B223-B235, wherein the surfactant is present (suitably where the composition comprises greater than or equal to 85 wt % water) at a concentration of 0.2-0.6 wt % of the overall composition.
B240. The composition of B1-B235, wherein the composition comprises at most 1 wt % surfactant(s), suitably at most 1 wt % of any surfactants defined in any of B223-B235.
B241. The composition of B1-B235, wherein the composition comprises at most 0.1 wt % surfactant(s), suitably at most 0.1 wt % of any surfactants defined in any of B223-B235.
B242. The composition of B1-B235, wherein the composition comprises at most 0.01 wt % surfactant(s), suitably at most 0.01 wt % of any surfactants defined in any of B223-B235.
B243. The composition of B1-B235, wherein the composition comprises at most 0.001 wt % surfactant(s), suitably at most 0.001 wt % of any surfactants defined in any of B223-B235.
B244. The composition of B1-B222, wherein the composition is free of surfactants, suitably free of any surfactants defined in any of B223-B235.

### Additional Oils

B245. The composition of B203-B244, wherein the one or more additional ingredients comprise one or more oils.
B246. The composition of B245, wherein the oils comprise or consist of a natural oil.
B247. The composition of B245-B246, wherein the oils comprise or consist of an organic oil (suitably animal or vegetable oil).
B248. The composition of B247, wherein the organic oil is a vegetable oil (usually rich in mono, di, and/or triglycerides) selected from the group consisting of (or any combination thereof):
Common vegetable oils: Coconut oil, Corn oil, Cottonseed oil, Olive oil, Palm oil, Peanut oil (Ground nut oil), Rapeseed oil (including Canola oil), Safflower oil, Sesame oil, Soybean oil, Sunflower oil;
Nut oils: Almond oil, Beech nut oil, Brazil nut oil, Cashew oil, Hazelnut oil, Macadamia oil, Mongongo nut oil, Pecan oil, Pine nut oil, Pistachio oil, Walnut oil;
Citrus oils: Grapefruit seed oil, Lemon oil, Orange oil, Cucurbitaceae oils: Bitter gourd oil, Bottle gourd oil, Buffalo gourd oil, Butternut squash seed oil, Egusi seed oil, Pumpkin seed oil, Watermelon seed oil;
Food Supplement oils: Agai oil, Black seed oil, Blackcurrant seed oil, Borage seed oil, Evening primrose oil, Flaxseed oil;
Other Edible oils: Amaranth oil, Apricot oil, Apple seed oil, Argan oil, Avocado oil, Babassu oil, Ben oil, Borneo tallow nut oil, Cape chestnut oil, Carob pod oil (Algaroba oil)Cocoa butter, Cocklebur oil, Cohune oil, Coriander seed oil, Date seed oil, Dika oil, False flax oil, Grape seed oil, Hemp oil, Kapok seed oil, Kenaf seed oil, Lallemantia oil, Mafura oil, Marula oil, Meadowfoam seed oil, Mustard oil (pressed), Niger seed oil, Poppyseed oil, Nutmeg butter, Okra seed oil, Papaya seed oil, Perilla seed oil, Persimmon seed oil, Pequi oil, Pili nut oil, Pomegranate seed oil, Poppyseed oil, Pracaxi oil, Virgin pracaxi oil, Prune kernel oil, Quinoa oil, Ramtil oil, Rice bran oil, Royle oil, Shea nuts, Sacha inchi oil, Sapote oil, Seje oil, Shea butter, Taramira oil, Tea seed oil (Camellia oil), Thistle oil, Tigernut oil (or nut-sedge oil), Tobacco seed oil, Tomato seed oil, Wheat germ oil;
Multi-purpose oils: Castor oil, Coconut oil (copra oil), Colza oil, Corn oil, Cottonseed oil, False flax oil, Hemp oil, Mustard oil, Palm oil, Peanut oil, Radish oil, Rapeseed oil, Ramtil oil, Rice bran oil, Safflower oil, Salicornia oil, Soybean oil, Sunflower oil, Tigernut oil, Tung oil;
Inedible oils: Copaiba, Jatropha oil, Jojoba oil, Milk bush, Nahor oil, Paradise oil, Petroleum nut oil, Pongamia oil (also known as Honge oil);
Drying oils: Dammar oil, Linseed oil, Poppyseed oil, Stillingia oil, Tung oil, Vernonia oil; and
Miscellaneous vegetable oils: Amur cork tree fruit oil, Artichoke oil, Astrocaryum murumuru butter, Balanos oil, Bladderpod oil, Brucea javanica oil, Burdock oil (Bur oil), Buriti oil, Candlenut oil (Kukui nut oil), Carrot seed oil (pressed), Castor oil, Chaulmoogra oil, Crambe oil, Croton oil (tiglium oil), Cuphea oil, Cupuagu butter, Honesty oil, Illipe butter, Jojoba oil, Mango oil, Mowrah butter, Neem oil, Ojon oil, Passion fruit oil, Rose hip seed oil, Rubber seed oil, Sea buckthorn oil, Sea rocket seed oil, Snowball seed oil (Viburnum oil), Tall oil, Tamanu or foraha oil, Tonka bean oil (Cumaru oil), Tucuma butter, Ucuhuba seed oil, moringa oil.
B249. The composition of B248, wherein the organic oil is a moringa oil, coconut oil, sunflower oil, avocado oil, or any combination thereof.
B250. The composition of B245-B249, when dependent upon B141 or any paragraph dependent thereon, wherein the weight ratio of stipulated glyceride(s) to oil(s) is 99:1 to 30:70.
B251. The composition of B250, wherein the weight ratio of stipulated glyceride(s) to oil(s) is 95:5 to 50:40.
B252. The composition of B250, wherein the weight ratio of stipulated glyceride(s) to oil(s) is 90:10 to 60:40.

### Additional Bile Acids or Bile Salts

B253. The composition of B203-B252, wherein the one or more additional ingredients comprise individual or mixtures of bile acids and/or bile salts.

### Absent/Low-Level Ingredients

B254. The composition of B1-B253, wherein the composition is (substantially, preferably entirely) free of polymers.
B255. The composition of B1-B253, wherein the composition is (substantially, preferably entirely) free of polyvinyl alcohol (PVA).
B256. The composition of B1-B253, wherein the composition is (substantially, preferably entirely) free of polyalkylene glycol(s) (e.g. polyethylene glycol and/or polypropylene glycol).
B257. The composition of B1-B253, wherein the composition is (substantially, preferably entirely) free of polyalkylene glycol ester(s) (e.g. polyethylene glycol and/or polypropylene glycol ester(s)).
B258. The composition of B1-B253, wherein the composition is (substantially, preferably entirely) free of any compounds that comprise polyalkylene glycol ester moieties (e.g. polyethylene glycol and/or polypropylene glycol ester moieties).
B259. The composition of B1-B253, wherein the composition is (substantially, preferably entirely) free of polyethylene glycol.
B260. The composition of B1-B253, wherein the composition is (substantially, preferably entirely) free of polyethylene glycol ester(s).
B261. The composition of B1-B253, wherein the composition is (substantially, preferably entirely) free of any compounds that comprise polyethylene glycol ester moieties.
B262. The composition of B1-B261, wherein the composition is (substantially, preferably entirely) free of surfactants.
B263. The composition of B1-B261, wherein the composition is (substantially, preferably entirely) free of poloxamer surfactants.
B264. The composition of B1-B261, wherein the composition is (substantially, preferably entirely) free of poloxamer 407 (e.g. Pluronic F127).
B265. The composition of B1-B261, wherein the composition is (substantially, preferably entirely) free of polysorbate surfactants.
B266. The composition of B1-B261, wherein the composition is (substantially, preferably entirely) free of polysorbate 20.
B267. The composition of B1-B261, wherein the composition is (substantially, preferably entirely) free of PEG-40 castor oil (e.g. Killiphor RH 40, Cremophor RH40) surfactants.
B268. The composition of B1-B261, wherein the composition is (substantially, preferably entirely) free of poloxamer and polysorbate surfactants.
B269. The composition of B1-B261, wherein the composition is (substantially, preferably entirely) free of poloxamer. polysorbate, and PEG-40 castor oil surfactants.
B270. The composition of B1-B261, wherein the composition comprises at most 3 wt % surfactant(s), suitably at most 3 wt % of any surfactants defined in any of B262-B269.
B271. The composition of B1-B261, wherein the composition comprises at most 1 wt % surfactant(s), suitably at most 1 wt % of any surfactants defined in any of B262-B269.
B272. The composition of B1-B261, wherein the composition comprises at most 0.1 wt % surfactant(s), suitably at most 0.1 wt % of any surfactants defined in any of B262-B269.
B273. The composition of B1-B261, wherein the composition comprises at most 0.01 wt % surfactant(s), suitably at most 0.01 wt % of any surfactants defined in any of B262-B269.
B274. The composition of B1-B261, wherein the composition comprises at most 0.001 wt % surfactant(s), suitably at most 0.001 wt % of any surfactants defined in any of B262-B269.
B275. The composition of B1-B274, wherein the composition is (substantially, preferably entirely) free of lipids other than those stipulated.
B276. The composition of B1-B275, wherein the composition is (substantially, preferably entirely) free of solvents (i.e. compounds that are liquids at SATP) other than water. Preferably, the composition is substantially (preferably entirely) free of organic solvents (suitably whether protic, polar, or unpolar).
   Structure, State. Properties of Metal Complex/Glycerides
B277. The composition of B141-B198 or any preceding paragraph dependent thereon, insofar as it depends on B1-B85 (i.e. such that the composition comprises both a metal complex and glyceride(s)), wherein the metal complex (or relevant sub-definition(s) thereof) is solubilised and/or encapsulated within the glyceride(s) (or relevant sub-definition(s) thereof).
B278. The composition of B277, wherein the metal complex is dissolved within the glyceride(s).
B279. The composition of B277-B278, wherein the metal complex is encapsulated within the glyceride(s).
B280. The composition of B277-B279, wherein the metal complex is both dissolved within and encapsulated within the glyceride(s).
B281. The composition of B277-B280, wherein the composition comprises lamella structures comprising both the metal complex and the glyceride(s). Lamella structures are especially predominant in dehydrated (or low water content) systems.
B282. The composition of B281, wherein the composition is non-transparent (i.e. is opaque) at SATP. This is generally due to a prevailing non-isotropic structure (e.g. lamella structure).
B283. The composition of B281-B282, wherein the composition is non-transparent (i.e. is opaque) at temperatures below the melting point of either the composition or of the predominant lipid or glyceride(s) and is transparent at temperatures above the melting point of either the composition or of the predominant lipid or glyceride(s).
B284. The composition of B281-B283, wherein the composition becomes transparent upon melting (e.g. when heated at least 5° C. above the melting point of either the composition or of the predominant lipid or glyceride(s)). In dry form (i.e. where the composition contains substantially no water), the lipid/glyceride(s) melt around 35-37° C. and become transparent upon melting. In wet form (i.e. where the composition contains substantial amounts of water), the composition suitably adopts a cubic phase even below the melting point, which is substantially transparent.

Such a cubic phase general melts above 70-80° C. and changes phase but remains transparent. In some cases, prior to melting (but at higher temperatures), the cubic phase converts into a hexagonal phase, which is opaque.
B285. The composition of B281-B284, wherein the lamella structures are characterised by a liquid crystalline fluid lamellar phase comprising both the metal complex and the glyceride(s).
B286. The composition of B281-B285, wherein one or more entire molecules of the metal complex are contained within a single lamella that also contains the glyceride(s).
B287. The composition of B281-B286, wherein one or more molecules of the metal complex bridge between two separate lamellae which each contains the glyceride(s) (i.e. such molecules of the metal complex are only partially contained within a single lamella).
B288. The composition of B277-B279, wherein the composition comprises both the metal complex and the glyceride(s) in a cubic phase, preferably a bicontinuous cubic Pn3m phase. Such cubic phases are especially predominant in hydrated (water-containing, or high water content, especially >20 wt %) systems.
B289. The composition of B288, wherein the composition comprises some lamella structures comprising both the metal complex and the glyceride(s).
B290. The composition of B288-B289, wherein the composition is non-transparent at SATP. This is generally due to a prevailing non-isotropic structure of the lamella structures.
B291. The composition of B288-B290, wherein the composition is or becomes transparent at 40° C. or higher, suitably at 30° C. or higher. This is suitably due to lamella structures being converted back into a bicontinuous cubic Pn3m phase, which is substantially isotropic. This transparent behaviour generally holds until very high metal complex contents are reached, at which point the compositions become more opaque.

### Concentration Metal Complex

B292. The composition of B1-B85 or any preceding paragraph dependent thereon, wherein the composition comprises 0.0001-50 wt % metal complex.
B293. The composition of B1-B85 or any preceding paragraph dependent thereon, wherein the composition comprises 0.001-40 wt % metal complex.
B294. The composition of B1-B85 or any preceding paragraph dependent thereon, wherein the composition comprises 0.005-30 wt % metal complex.
B295. The composition of B1-B85 or any preceding paragraph dependent thereon, wherein the composition (especially where the composition has no or low water content, and little to no encapsulation of the metal complex) comprises 0.0001-1 wt % metal complex.
B296. The composition of B1-B85 or any preceding paragraph dependent thereon, wherein the composition (especially where the composition has no or low water content, and little to no encapsulation of the metal complex) comprises 0.001-0.1 wt % metal complex.
B297. The composition of B1-B85 or any preceding paragraph dependent thereon, wherein the composition (especially where the composition has no or low water content, and little to no encapsulation of the metal complex) comprises 0.005-0.05 wt % metal complex.
B298. The composition of B1-B85 or any preceding paragraph dependent thereon, wherein the composition (especially where the composition has no or low water content, and little to no encapsulation of the metal complex) comprises 0.01 wt % metal complex.
B299. The composition of B1-B85 or any preceding paragraph dependent thereon, wherein the composition (especially where the composition has no or low water content, and at least some encapsulation of the metal complex) comprises 1-30 wt % metal complex.
B300. The composition of B1-B85 or any preceding paragraph dependent thereon, wherein the composition (especially where the composition has no or low water content, and at least some encapsulation of the metal complex) comprises 2-20 wt % metal complex.
B301. The composition of B1-B85 or any preceding paragraph dependent thereon, wherein the composition (especially where the composition has no or low water content, and at least some encapsulation of the metal complex) comprises 5-15 wt % metal complex.
B302. The composition of B1-B85 or any preceding paragraph dependent thereon, wherein the composition (especially where the composition has no or low water content, and at least some encapsulation of the metal complex) comprises 10-12 wt % metal complex.
B303. The composition of B1-B85 or any preceding paragraph dependent thereon, wherein the composition (especially where the composition has at least some water, but little to no encapsulation of the metal complex) comprises 0.01-20 wt % metal complex.
B304. The composition of B1-B85 or any preceding paragraph dependent thereon, wherein the composition (especially where the composition has at least some water, but little to no encapsulation of the metal complex) comprises 0.1-15 wt % metal complex.
B305. The composition of B1-B85 or any preceding paragraph dependent thereon, wherein the composition (especially where the composition has at least some water, but little to no encapsulation of the metal complex) comprises 1-10 wt % metal complex.
B306. The composition of B1-B85 or any preceding paragraph dependent thereon, wherein the composition (especially where the composition has at least some water, but little to no encapsulation of the metal complex) comprises 2-7 wt % metal complex.
B307. The composition of B1-B85 or any preceding paragraph dependent thereon, wherein the composition (especially where the composition has at least some water, but little to no encapsulation of the metal complex) comprises 1-3 wt % metal complex.
B308. The composition of B1-B85 or any preceding paragraph dependent thereon, wherein the composition (especially where the composition has at least some water, but little to no encapsulation of the metal complex) comprises 4-8 wt % metal complex.
B309. The composition of B1-B85 or any preceding paragraph dependent thereon, wherein the composition (especially where the composition has at least some water, and at least some encapsulation of the metal complex) comprises 1-50 wt % metal complex.
B310. The composition of B1-B85 or any preceding paragraph dependent thereon, wherein the composition (especially where the composition has at least some water, and at least some encapsulation of the metal complex) comprises 5-40 wt % metal complex.
B311. The composition of B1-B85 or any preceding paragraph dependent thereon, wherein the composition (especially where the composition has at least some water, and at least some encapsulation of the metal complex) comprises 10-30 wt % metal complex.
B312. The composition of B1-B85 or any preceding paragraph dependent thereon, wherein the composition (especially where the composition has at least some water, and at least some encapsulation of the metal complex) comprises 20-30 wt % metal complex.
B313. The composition of B1-B85 or any preceding paragraph dependent thereon, wherein the composition (especially where the composition has at least some water, and at least some encapsulation of the metal complex) comprises 23-26 wt % metal complex.
B314. The composition of B1-B85 or any preceding paragraph dependent thereon, wherein the composition (especially where the composition is aqueous) comprises 0.001-5 wt % metal complex.
B315. The composition of B1-B85 or any preceding paragraph dependent thereon, wherein the composition (especially where the composition is aqueous) comprises 0.01-1 wt % metal complex.
B316. The composition of B1-B85 or any preceding paragraph dependent thereon, wherein the composition (especially where the composition is aqueous) comprises 0.02-0.5 wt % metal complex.
B317. The composition of B1-B85 or any preceding paragraph dependent thereon, wherein the composition (especially where the composition is aqueous) comprises 0.02-0.06 wt % metal complex.
B318. The composition of B1-B85 or any preceding paragraph dependent thereon, wherein the composition (especially where the composition is aqueous) comprises 0.1-0.4 wt % metal complex.
B319. The composition of any of B292-B318, wherein the “metal complex” (whether defined generally, specifically, or at any level of intermediate generalisation therebetween) is substituted for a metal compound as defined in any of B124-B140, and suitably any concentrations given in relation to the metal complex apply to the metal compound.

### Ratio Metal Complex/Glycerides

B320. The composition of B141-B198 or any preceding paragraph dependent thereon, insofar as it depends on B1-B85 (i.e. such that the composition comprises both a metal complex and glyceride(s)—i.e. stipulated glycerides), wherein the metal complex (or relevant sub-definition(s) thereof) and the glyceride(s) (or relevant sub-definition(s) thereof) are present within the composition at a defined relative weight ratio.
B321. The composition of B320, wherein the weight ratio of the metal complex to the glyceride(s) is 0.01:1000-900:1000.
B322. The composition of B320, wherein the weight ratio of the metal complex to the glyceride(s) is 0.1:1000-700:1000.
B323. The composition of B320, wherein the weight ratio of the metal complex to the glyceride(s) is 0.5:1000-600:1000.
B324. The composition of B320, wherein (especially where the composition has no or low water content, and little to no encapsulation of the metal complex) the weight ratio of the metal complex to the glyceride(s) is 0.0001:1000-50:1000.
B325. The composition of B320, wherein (especially where the composition has no or low water content, and little to no encapsulation of the metal complex) the weight ratio of the metal complex to the glyceride(s) is 0.001:1000-10:1000.
B326. The composition of B320, wherein (especially where the composition has no or low water content, and little to no encapsulation of the metal complex) the weight ratio of the metal complex to the glyceride(s) is 0.1:1000-5:1000.
B327. The composition of B320, wherein (especially where the composition has no or low water content, and little to no encapsulation of the metal complex) the weight ratio of the metal complex to the glyceride(s) is 0.5:1000-1.5:1000.
B328. The composition of B320, wherein (especially where the composition has no or low water content, and at least some encapsulation of the metal complex) the weight ratio of the metal complex to the glyceride(s) is 0.1:1000-500:1000.
B329. The composition of B320, wherein (especially where the composition has no or low water content, and at least some encapsulation of the metal complex) the weight ratio of the metal complex to the glyceride(s) is 10:1000-400:1000.
B330. The composition of B320, wherein (especially where the composition has no or low water content, and at least some encapsulation of the metal complex) the weight ratio of the metal complex to the glyceride(s) is 50:1000-200:1000.
B331. The composition of B320, wherein (especially where the composition has no or low water content, and at least some encapsulation of the metal complex) the weight ratio of the metal complex to the glyceride(s) is 90:1000-160:1000.
B332. The composition of B320, wherein (especially where the composition has at least some water, but little to no encapsulation of the metal complex) the weight ratio of the metal complex to the glyceride(s) is 0.1:1000-300:1000.
B333. The composition of B320, wherein (especially where the composition has at least some water, but little to no encapsulation of the metal complex) the weight ratio of the metal complex to the glyceride(s) is 1:1000-200:1000.
B334. The composition of B320, wherein (especially where the composition has at least some water, but little to no encapsulation of the metal complex) the weight ratio of the metal complex to the glyceride(s) is 10:1000-150:1000.
B335. The composition of B320, wherein (especially where the composition has at least some water, but little to no encapsulation of the metal complex) the weight ratio of the metal complex to the glyceride(s) is 20:1000-130:1000.
B336. The composition of B320, wherein (especially where the composition has at least some water, but little to no encapsulation of the metal complex) the weight ratio of the metal complex to the glyceride(s) is 10:1000-70:1000.
B337. The composition of B320, wherein (especially where the composition has at least some water, but little to no encapsulation of the metal complex) the weight ratio of the metal complex to the glyceride(s) is 50:1000-150:1000.
B338. The composition of B320, wherein (especially where the composition has at least some water, and at least some encapsulation of the metal complex) the weight ratio of the metal complex to the glyceride(s) is 10:1000-900:1000.
B339. The composition of B320, wherein (especially where the composition has at least some water, and at least some encapsulation of the metal complex) the weight ratio of the metal complex to the glyceride(s) is 50:1000-700:1000.
B340. The composition of B320, wherein (especially where the composition has at least some water, and at least some encapsulation of the metal complex) the weight ratio of the metal complex to the glyceride(s) is 100:1000-600:1000.
B341. The composition of B320, wherein (especially where the composition has at least some water, and at least some encapsulation of the metal complex) the weight ratio of the metal complex to the glyceride(s) is 200:1000-580:1000.
B342. The composition of B320, wherein (especially where the composition has at least some water, and at least some encapsulation of the metal complex) the weight ratio of the metal complex to the glyceride(s) is 500:1000-550:1000.
B343. The composition of B320, wherein (especially where the composition is aqueous) the weight ratio of the metal complex to the glyceride(s) is 0.1:1000-600:1000.
B344. The composition of B320, wherein (especially where the composition is aqueous) the weight ratio of the metal complex to the glyceride(s) is 1:1000-200:1000.
B345. The composition of B320, wherein (especially where the composition is aqueous) the weight ratio of the metal complex to the glyceride(s) is 5:1000-100:1000.
B346. The composition of B320, wherein (especially where the composition is aqueous) the weight ratio of the metal complex to the glyceride(s) is 2:1000-20:1000.
B347. The composition of B320, wherein (especially where the composition is aqueous) the weight ratio of the metal complex to the glyceride(s) is 30:1000-70:1000.
B348. The composition of any of B320-B347, wherein the “metal complex” (whether defined generally, specifically, or at any level of intermediate generalisation therebetween) is substituted for a metal compound as defined in any of B124-B140, and suitably any ratios given in relation to the metal complex apply to the metal compound.

### Concentration of Other Glyceride(s)/Lipids

B349. The composition of B141-B198 or any preceding paragraph dependent thereon, insofar as it depends on B1-B85 (i.e. such that the composition comprises both a metal complex and glyceride(s)), wherein the stipulated (i.e. the defined) glyceride(s) (or relevant sub-definition(s) thereof) are the predominant glyceride(s), and suitably the predominant lipid(s) (e.g. fatty-acid-containing compounds), within the composition. Suitably, all glyceride(s) (including predominant glyceride(s)) and lipids within the composition are collectively termed lipid compounds.
B350. The composition of B141-B198 or any preceding paragraph dependent thereon, insofar as it depends on B1-B85 (i.e. such that the composition comprises both a metal complex and glyceride(s)), wherein the stipulated (i.e. the defined) glyceride(s) (or relevant sub-definition(s) thereof) constitute at least 50 wt % of the total amount of glyceride(s) present within the composition (i.e. the “50 wt %” is relative to total glyceride(s) rather than the composition as a whole), and preferably constitute at least 50 wt % of the total amount of lipid(s) present within the composition (i.e. the “50 wt %” is relative to total lipid(s) rather than the composition as a whole). All glyceride(s) (including predominant glyceride(s)) and other lipids within the composition are collectively termed lipid compounds or just “lipids”.
B351. The composition of B350, wherein the stipulated (or defined) glyceride(s) constitute at least 70 wt % of the total amount of glyceride(s) present within the composition.
B352. The composition of B350, wherein the stipulated (or defined) glyceride(s) constitute at least 85 wt % of the total amount of glyceride(s) present within the composition.
B353. The composition of B350, wherein the stipulated (or defined) glyceride(s) constitute at least 89 wt % of the total amount of glyceride(s) present within the composition.
B354. The composition of B350, wherein the stipulated (or defined) glyceride(s) constitute at least 95 wt % of the total amount of glyceride(s) present within the composition.
B355. The composition of B350, wherein the stipulated (or defined) glyceride(s) constitute at least 70 wt % of the total amount of lipids present within the composition.
B356. The composition of B350, wherein the stipulated (or defined) glyceride(s) constitute at least 85 wt % of the total amount of lipids present within the composition.
B357. The composition of B350, wherein the stipulated (or defined) glyceride(s) constitute at least 89 wt % of the total amount of lipids present within the composition.
B358. The composition of B350, wherein the stipulated (or defined) glyceride(s) constitute at least 95 wt % of the total amount of lipids present within the composition.
B359. The composition of B1-B358, wherein glyceride(s) (stipulated and/or unstipulated) constitute at least 70 wt % of the total amount of lipids present within the composition.
B360. The composition of B359, wherein glyceride(s) (stipulated and/or unstipulated) constitute at least 85 wt % of the total amount of lipids present within the composition.
B361. The composition of B360, wherein glyceride(s) (stipulated and/or unstipulated) constitute at least 95 wt % of the total amount of lipids present within the composition.
B362. The composition of B361, wherein glyceride(s) (stipulated and/or unstipulated) constitute at least 99 wt % of the total amount of lipids present within the composition.

### Concentration of Water

B363. The composition of B199 or any preceding paragraph dependent thereon (particularly those that contain both metal complex and glyceride(s)), wherein the composition comprises 0-99 wt % water.
B364. The composition of B363, wherein the composition comprises 0.0001-98 wt % water.
B365. The composition of B363, wherein the composition comprises 0.001-95 wt % water.
B366. The composition of B363, wherein the composition comprises less than or equal to 80 wt % water (i.e. the composition has at most 80 wt % water, and may contain 0 wt % water).
B367. The composition of B363, wherein the composition comprises less than or equal to 60 wt % water (i.e. the composition has at most 60 wt % water, and may contain 0 wt % water).
B368. The composition of B363, wherein the composition comprises less than or equal to 40 wt % water (i.e. the composition has at most 40 wt % water, and may contain 0 wt % water).
B369. The composition of B363, wherein the composition (especially where the composition has no or low water content, and either little to no encapsulation or at least some encapsulation of the metal complex) comprises less than or equal to 5 wt % water (i.e. the composition has at most 5 wt % water, and may contain 0 wt % water).
B370. The composition of B363, wherein the composition (especially where the composition has no or low water content, and either little to no encapsulation or at least some encapsulation of the metal complex) comprises less than or equal to 2 wt % water.
B371. The composition of B363, wherein the composition (especially where the composition has no or low water content, and either little to no encapsulation or at least some encapsulation of the metal complex) comprises less than or equal to 1 wt % water.
B372. The composition of B363, wherein the composition (especially where the composition has no or low water content, and either little to no encapsulation or at least some encapsulation of the metal complex) comprises 0 wt % water.
B373. The composition of B363, wherein the composition (especially where the composition has at least some water, but little to no encapsulation of the metal complex) comprises 10-60 wt % water.
B374. The composition of B363, wherein the composition (especially where the composition has at least some water, but little to no encapsulation of the metal complex) comprises 15-60 wt % water.
B375. The composition of B363, wherein the composition (especially where the composition has at least some water, but little to no encapsulation of the metal complex) comprises 25-50 wt % water.
B376. The composition of B363, wherein the composition (especially where the composition has at least some water, but little to no encapsulation of the metal complex) comprises 30-45 wt % water.
B377. The composition of B363, wherein the composition (especially where the composition has at least some water, but little to no encapsulation of the metal complex) comprises 35-40 wt % water.
B378. The composition of B363, wherein the composition (especially where the composition has at least some water, and at least some encapsulation of the metal complex) comprises 10-60 wt % water.
B379. The composition of B363, wherein the composition (especially where the composition has at least some water, and at least some encapsulation of the metal complex) comprises 10-50 wt % water.
B380. The composition of B363, wherein the composition (especially where the composition has at least some water, and at least some encapsulation of the metal complex) comprises 20-40 wt % water.
B381. The composition of B363, wherein the composition (especially where the composition has at least some water, and at least some encapsulation of the metal complex) comprises 25-35 wt % water.
B382. The composition of B363, wherein the composition (especially where the composition has at least some water, and at least some encapsulation of the metal complex) comprises 28-32 wt % water.
B383. The composition of B373-B382, wherein the composition is formed by adding a required amount of water to a composition of B369-B372, and suitably thereafter mixing, suitably with sufficient heating to melt the composition of B369-B372 or at least the glyceride(s) thereof. Wherever metal complex is to be added (e.g. to form a hydrated composition containing significant quantities of metal complex), suitably metal complex addition is performed by mixing additional metal complex with the composition of B369-B372 (preferably heated so as to be in a melted state) before the addition of water. Products are suitably stored at or below 20° C.
B384. The composition of B363, wherein the composition (especially where the composition is aqueous) comprises 85-99.9 wt % water. Such compositions may suitable include a surfactant.
B385. The composition of B363, wherein the composition (especially where the composition is aqueous) comprises 85-99 wt % water. Such compositions may suitable include a surfactant.
B386. The composition of B363, wherein the composition (especially where the composition is aqueous) comprises 70-98 wt % water. Such compositions may suitable include a surfactant.
B387. The composition of B363, wherein the composition (especially where the composition is aqueous) comprises 90-97 wt % water. Such compositions may suitable include a surfactant.
B388. The composition of B363, wherein the composition (especially where the composition is aqueous) comprises 93-96 wt % water. Such compositions may suitable include a surfactant.
B389. The composition of B384-B388, wherein the composition is formed by adding a required amount of water to a composition of B369-B372, and suitably thereafter mixing, suitably with sufficient heating to melt the composition of B369-B372 or at least the glyceride(s) thereof.

### Additional Composition-Specific Facets

B390. The composition of B1-B389, wherein at least 1 wt % of the composition is constituted by the stipulated ingredients.
B391. The composition of B1-B389, wherein at least 10 wt % of the composition is constituted by the stipulated ingredients.
B392. The composition of B1-B389, wherein at least 15 wt % of the composition is constituted by the stipulated ingredients.
B393. The composition of B1-B389, wherein at least 20 wt % of the composition is constituted by the stipulated ingredients.
B394. The composition of B1-B389, wherein at least 50 wt % of the composition is constituted by the stipulated ingredients.
B395. The composition of B1-B389, wherein at least 70 wt % of the composition is constituted by the stipulated ingredients.
B396. The composition of B1-B389, wherein at least 80 wt % of the composition is constituted by the stipulated ingredients.
B397. The composition of B1-B389, wherein at least 85 wt % of the composition is constituted by the stipulated ingredients.
B398. The composition of B1-B389, wherein at least 90 wt % of the composition is constituted by the stipulated ingredients.
B399. The composition of B1-B389, wherein at least 95 wt % of the composition is constituted by the stipulated ingredients.
B400. The composition of B1-B389, wherein at least 99 wt % of the composition is constituted by the stipulated ingredients.
B401. The composition of B1-B389, wherein the composition consists of the stipulated ingredients.
B402. The composition of B1-B389, wherein at least 1 wt % of the composition is constituted by the stipulated metal complex and stipulated glyceride(s) (e.g. as per the glyceride(s) stipulated in B141-B198).
B403. The composition of B1-B389, wherein at least 10 wt % of the composition is constituted by the stipulated metal complex and stipulated glyceride(s) (e.g. as per the glyceride(s) stipulated in B141-B198).
B404. The composition of B1-B389, wherein at least 15 wt % of the composition is constituted by the stipulated metal complex and stipulated glyceride(s) (e.g. as per the glyceride(s) stipulated in B141-B198).
B405. The composition of B1-B389, wherein at least 20 wt % of the composition is constituted by the stipulated metal complex and stipulated glyceride(s) (e.g. as per the glyceride(s) stipulated in B141-B198).
B406. The composition of B1-B389, wherein at least 50 wt % of the composition is constituted by the stipulated metal complex and stipulated glyceride(s) (e.g. as per the glyceride(s) stipulated in B141-B198).
B407. The composition of B1-B389, wherein at least 70 wt % of the composition is constituted by the stipulated metal complex and stipulated glyceride(s) (e.g. as per the glyceride(s) stipulated in B141-B198).
B408. The composition of B1-B389, wherein at least 80 wt % of the composition is constituted by the stipulated metal complex and stipulated glyceride(s) (e.g. as per the glyceride(s) stipulated in B141-B198).
B409. The composition of B1-B389, wherein at least 85 wt % of the composition is constituted by the stipulated metal complex and stipulated glyceride(s) (e.g. as per the glyceride(s) stipulated in B141-B198).
B410. The composition of B1-B389, wherein at least 90 wt % of the composition is constituted by the stipulated metal complex and stipulated glyceride(s) (e.g. as per the glyceride(s) stipulated in B141-B198).
B411. The composition of B1-B389, wherein at least 95 wt % of the composition is constituted by the stipulated metal complex and stipulated glyceride(s) (e.g. as per the glyceride(s) stipulated in B141-B198).
B412. The composition of B1-B389, wherein at least 99 wt % of the composition is constituted by the stipulated metal complex and stipulated glyceride(s) (e.g. as per the glyceride(s) stipulated in B141-B198).
B413. The composition of B1-B412, wherein centrifugation (e.g. 3000 rpm for 5 min at RT) of the composition yields less than 10 wt % sedimentation (i.e. less than 10 wt % of the composition as a whole).
B414. The composition of B1-B412, wherein centrifugation (e.g. 3000 rpm for 5 min at RT) of the composition yields less than 5 wt % sedimentation (i.e. less than 5 wt % of the composition as a whole).
B415. The composition of B1-B412, wherein centrifugation (e.g. 3000 rpm for 5 min at RT) of the composition yields less than 2 wt % sedimentation (i.e. less than 2 wt % of the composition as a whole).
B416. The composition of B1-B412, wherein centrifugation (e.g. 3000 rpm for 5 min at RT) of the composition yields less than 1 wt % sedimentation (i.e. less than 1 wt % of the composition as a whole).
B417. The composition of B1-B412, wherein centrifugation (e.g. 3000 rpm for 5 min at RT) of the composition yields less than 0.1 wt % sedimentation (i.e. less than 0.1 wt % of the composition as a whole).
B418. The composition of B1-B412, wherein centrifugation (e.g. 3000 rpm for 5 min at RT) of the composition yields less than 0.01 wt % sedimentation (i.e. less than 0.01 wt % of the composition as a whole).
B419. The composition of B1-B412, wherein centrifugation (e.g. 3000 rpm for 5 min at RT) of the composition yields (substantially) no sedimentation.
B420. The composition of B1-B412, wherein centrifugation (e.g. 3000 rpm for 5 min at RT) of the composition yields either no sedimentation or sedimentation containing less than 50 wt % of the metal complex present within the composition (i.e. this refers to 50 wt % of the total amount of metal complex, not 50 wt % of the composition as a whole).
B421. The composition of B420, wherein centrifugation (e.g. 3000 rpm for 5 min at RT) of the composition yields either no sedimentation or sedimentation containing less than 20 wt % of the metal complex present within the composition.
B422. The composition of B420, wherein centrifugation (e.g. 3000 rpm for 5 min at RT) of the composition yields either no sedimentation or sedimentation containing less than 10 wt % of the metal complex present within the composition.
B423. The composition of B420, wherein centrifugation (e.g. 3000 rpm for 5 min at RT) of the composition yields either no sedimentation or sedimentation containing less than 5 wt % of the metal complex present within the composition.
B424. The composition of B420, wherein centrifugation (e.g. 3000 rpm for 5 min at RT) of the composition yields either no sedimentation or sedimentation containing less than 1 wt % of the metal complex present within the composition.
B425. The composition of B420, wherein centrifugation (e.g. 3000 rpm for 5 min at RT) of the composition yields either no sedimentation or sedimentation containing less than 0.1 wt % of the metal complex present within the composition.
B426. The composition of B420, wherein centrifugation (e.g. 3000 rpm for 5 min at RT) of the composition yields either no sedimentation of any metal complex present within the composition.
B427. The composition of B1-B426, wherein the composition is a solid, fluid, viscoelastic or semi-solid.

The definitions set forth in any of paragraphs B1-B427 may be applied (especially where compatible within the given context) to any of the specific embodiments set forth in A1-A101 or indeed any other definitions, embodiments, or aspects set forth herein in relation to the composition.

### Examples

### Materials and Equipment

The following materials and equipment were used: Copper aspirinate (CuASP), copper salicylate (CuSL), monoolein (MO) (Croda Europe Ltd.), monolinolein (ML) (Sigma-Aldrich, UK), Dimodan-U (DU) (mixture of MO and ML) (Danisco (UK) Limited).

### Synthesis of Copper (II) Aspirinate (CuASP)

1.68 g (20 mmol) of sodium bicarbonate was dissolved in 30 mL of deionized water, to that 3.60 g (20 mmol) of Aspirin was added and allowed to be dissolved in it by stirring. 2.50 g (10 mmol) of copper sulphate dissolved in 20 mL of distilled water was added slowly into the above solution with constant stirring. Evolution of CO₂ was noted (Fujimori, T et al. “Orally active antioxidative copper(II) aspirinate: synthesis, structure characterization, superoxide scavenging activity, and in vitro and in vivo antioxidative evaluations.” Journal of Biological Inorganic Chemistry, Vol. 10, 8 (2005): 831-41. doi:10.1007/s00775-005-0031-3). The precipitate formed was collected using Buchner funnel and dried under vacuum.

### Synthesis of Copper (II) Salicylate (CuSL)

2.76 g (20 mmol) of salicylic acid was dissolved in 45 mL 33% ethyl alcohol. This solution was heated to 1200° C. and 2.22 g (10 mmol) basic copper carbonate was added (Evolution of CO₂ was noted). The resultant slurry was refluxed under oil bath for 14 hours by maintained the temperature at 1200° C. After 14 hours the precipitate was washed several times with freshly prepared 33% ethyl alcohol. The precipitate formed was collected using Buchner funnel and dried under vacuum.

Chemicals required for above syntheses were obtained from Sigma-Aldrich, UK.

### Analytical Protocols

The following analytical protocols were used: The SAXSpace instrument (Anton Paar, Graz, Austria) was utilized for Small angle X-ray scattering (SAXS) studies. Sample bottle photographs were captured using Samsung A5 mobile phone camera. Sonics & Materials Vibra-Cell VCX750, Jencons, UK was utilized for ultrasonic preparation of emulsion samples.

### General Summary of Formulations Tested

TABLE 1
Formulations Tested

| | | | Maximum | | |
|---|---|---|---|---|---|
| | | | Copper | | |
| | | | complex | | |
| | | | content | | |
| | Name of | Formulation | (mg/g of | Proportion | |
| No. | Formulation | composition | lipid) | (wt %) | Comment |
| 1 | F01-CuASP- | CuASP + MO | 1.0 ± 0.5 | a = 0.01%, b = | Low solubility (transparent turquoise |
| | MO | | | 99.99% | colour at room temperature) |
| 2 | F01-CuASP- | CuASP + DU | 1.0 ± 0.5 | a = 0.01%, b = | Low solubility (transparent turquoise |
| | DU | | | 99.99% | colour at room temperature) |
| 3 | F01-CuASP- | CuASP + MO | 126.0 ± | a = 11.2%, b = | Encapsulation -non-transparent mixture |
| | MO-E | | 20 | 88.8% | (stays homogeneous in lipid system but |
| | | | | | sometimes separates if centrifuged |
| | | | | | and/or kept in molten condition for |
| | | | | | longer) |
| 4 | F01-CuASP- | CuASP + DU | 130.0 ± | a = 11.5%, b = | Encapsulation -non-transparent mixture |
| | DU-E | | 20 | 88.5% | (stays homogeneous in lipid system but |
| | | | | | sometimes separates if centrifuged |
| | | | | | and/or kept in molten condition for |
| | | | | | longer) |
| 5 | F02-CuASP- | CuASP + DU + water | 106.0 ± | a = 6.0%, b = | Good solubility (transparent turquoise |
| | DU | | 15 | 56.4%, c = | coloured viscous gel) |
| | | | | 37.6% | |
| 6 | F02-CuASP- | CuASP + MO + water | 533 ± 40 | a = 24.2%, b = | Encapsulation -non-transparent mixture |
| | DU-E | | | 45.5%, c = | (stays homogeneous in lipid system but |
| | | | | 30.3% | sometimes separates if centrifuged). |
| 7 | F03-CuASP- | CuASP + DU + aqueous | 50.0 ± 5 | a = 0.25%, b = | Particulate oil-in-water emulsion is |
| | DU | F127 solution | | 4.96%, c = | stable |
| | | | | 94.32%, d = | |
| | | | | 0.47% | |
| 8 | F01-CuSL- | CuSL + MO | 1.0 ± 0.5 | a = 0.01%, b = | Low solubility (transparent dark green |
| | MO | | | 99.99% | colour at room temperature) |
| 9 | F01-CuSL- | CuSL + DU | 1.0 ± 0.5 | a = 0.01%, b = | Low solubility (transparent dark green |
| | DU | | | 99.99% | colour at room temperature) |
| 10 | F01-CuSL- | CuSL + MO | 120.0 ± | a = 10.7%, b = | Encapsulation -non-transparent mixture |
| | MO-E | | 25 | 89.3% | (stays homogeneous in lipid system but |
| | | | | | sometimes separates if centrifuged |
| | | | | | and/or kept in molten condition for |
| | | | | | longer). |
| 11 | F01-CuSL- | CuSL + DU | 128.0 ± | a = 11.3%, b = | Encapsulation -non-transparent mixture |
| | DU-E | | 25 | 88.7% | (stays homogeneous in lipid system but |
| | | | | | sometimes separates if centrifuged |
| | | | | | and/or kept in molten condition for |
| | | | | | longer). |
| 12 | F02-CuSL- | CuSL + DU + water | 38.0 ± 6 | a = 2.2%, b = | Full solubility (transparent green colour |
| | DU | | | 58.7%, c = | at room temperature) |
| | | | | 39.1% | |
| 13 | F02-CuSL- | CuSL + DU + water | 533 ± 40 | a = 24.2%, b = | Encapsulation -non-transparent mixture |
| | DU-E | | | 45.5%, c = | (stays homogeneous in lipid system but |
| | | | | 30.3% | sometimes separates if centrifuged). |
| 14 | F03-CuSL- | CuSL + DU + aqueous | 8 ± 2 | a = 0.04%, b = | Particulate oil-in-water emulsion is |
| | DU | F127 solution | | 4.97%, c = | stable. |
| | | | | 94.51%, d = | |
| | | | | 0.47% | |

a = copper complex,
b = lipid,
c = water,
d = surfactant stabilizer
CuASP: copper aspirinate,
CuSL: copper salicylate

The compositions of Table 1 were prepared as set forth hereinafter so as to afford the indicated wt % proportions of each respective ingredient, and to produce compositions containing the indicated weight ratio of mg of copper complex per g of lipid(s).

Formulations of the invention involve lipid-based formulations that are capable of solubilizing and/or encapsulating high concentrations of copper complex (or derivatives thereof). The formulations contain lipids, preferably one or more monoglycerides containing at least one unsaturated moiety (preferably C₈-C₂₀, preferably C₁₈, preferably oleate and/or linoleate). The formulations can have a water content in the range of 0 to 95% (w/w), sometimes higher water content for aqueous dispersions. The formulation include copper complex (or derivatives thereof) in solubilized and/or encapsulated form, generally in a concentration range of 0.5-800 mg/g of lipid. In general, such formulations do not require surfactant stabilization, though in some embodiments they may. The formulations may include an optional oil (e.g. food-based) or other component to fine-tune physical form/s of the formulation(s). The formulations generally contain only ingredients that are generally recognized as safe (GRAS). The formulations generally have no need of any organic solvent, be it during preparation or for stabilisation (e.g. during storage). The degree of copper complex loading within formulations can be modulated by altering the formulation composition, particularly, by adding various concentrations of additives including one or more natural oils, other lipids, fatty acids, bile salts (and/or bile acid(s)), hydrophilic molecules, hydrophobic molecules, and amphiphilic molecules. The physical state of the formulations can be modified into solid, fluid, and viscoelastic or semi-solid. The formulations can be tuned into various applicable forms including gel, cream, paste, dry film, powder, tablets, capsules, jellies, pastilles, gums, lotions, oil-in-water emulsion, water-in-oil emulsion and drops. The formulation can be suitable for all modes of administration into the body, preferably topical and oral mode of delivery.

The formulations (or derivatives thereof) can be useful for preparing material science and/or engineering products with antimicrobial and/or aesthetic properties.

Three different sets of formulations based on monoglycerides and mixtures in non-aqueous (F01), liquid crystalline bulk (F02) and nanostructured dispersion forms (F03) were prepared to obtain highly enhanced solubility, encapsulation and bioavailability of copper complexes for various material science, engineering, and medicinal applications. Entries in the “Name of Formulation” column of Table 1 are prefixed with the relevant Formulation Code. (F01, F02, F03) to indicate from which base formulation they are derived/varied.

### Example 1—Copper Complex Formulations (F01): Non-Aqueous Lipid Systems

Monoolein (MO) or Dimodan-U (DU) were employed to prepare non-aqueous formulations (F01) to solubilize or encapsulate copper complexes (Table 1). DU is a commercial monoglyceride (MG) mixture containing about 96% distilled MGs of which 62% is ML and 25% is MO; remaining 4% constitute free fatty acids and diglycerides.

### F01 Preparation

### Method 1

F01 formulations were prepared as follows:
Copper complex (be it CuASP or CuSL), in the powder form, was mixed with either MO (for F01-MO formulations) or DU (for F01-DU formulations) in a 20 ml scintillation vial. A lipid phase can be formed of only MO or DU or it may can contain an optional other component utilized improve copper complex loading and/or fine-tune a physical form of the final formulation.
Mixture was heated using block heater or similar equipment to melt the lipid (around 40-45° C.).
Spatula or similar tools were used to blend the constituents until homogeneous.
Mixtures were heated again, if required, followed by homogenization by spatula.
Final formulation was stored at room temperature (around 20° C.) or in fridge (around 4° C.).

### Method 2

In an alternative method, the copper complex (be it CuASP or CuSL) may be mixed in powder form and ultimately homogenised with either MO (for F01-MO formulations) or DU (for F01-DU formulations) with a mortar pestle (10-30 min). This alternative method allowed for greater concentrations of copper complex to be achieved (especially relative to the concentration of the lipid).

FIG. 2 shows an image of a formulation containing CuASP solubilized in molten MO.

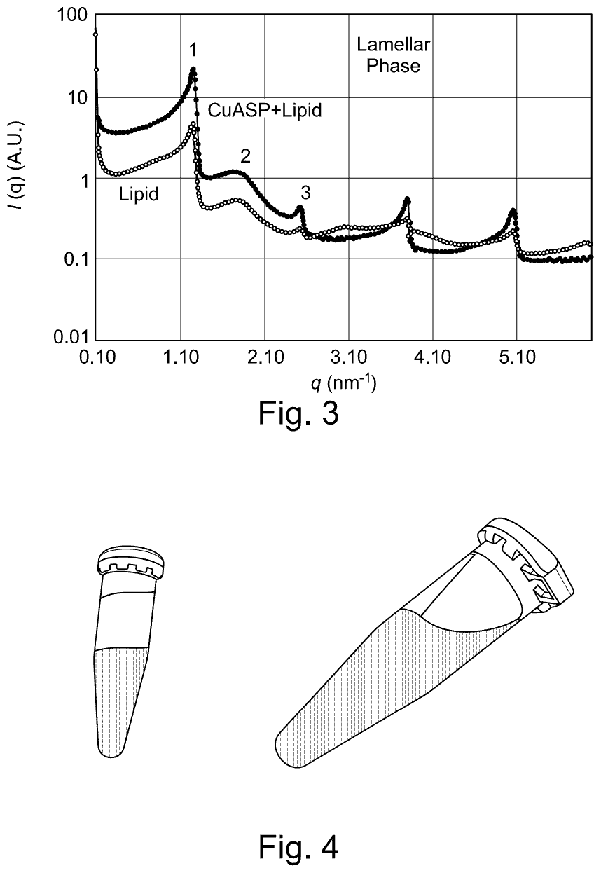

FIG. 3 shows is a trace showing small angle X-ray scattering patterns of pure lipid and CuASP-loaded lipid mixture (CuASP solubilized in molten MO+water). Points 1,2,3 . . . display characteristic peaks for lamellar phase for monoolein at room temperature.

### Copper Complex Solubility in MO and DU

Various formulations (at various copper aspirinate or copper salicylate concentrations) were prepared, as shown in Table 1, wherein maximum solubilities and/or encapsulation concentrations are shown.

Solubilized copper complex remains in this form for several weeks at room temperature.

### Properties of Copper Complex Formulations (F01)

F01-CuASP-MO had appealing turquoise colour all throughout the sample and a little higher typical viscosity than that of a molten cooking oil, at room temperature (FIG. 2). Small angle X-ray scattering (SAXS) patterns confirm the presence of lamellar phase for MO in dry state at room temperature exhibiting a typical lattice parameter of 49.8 Å (FIG. 3). The latter remain unchanged upon addition of CuASP.

### Copper Complex Encapsulation (F01-E Formulations)

FIG. 4 shows two images of a formulation containing CuASP encapsulated in DU (F01-CuASP-DU-E) at room temperature (Left) and molten state (Right).

Although CuASP has very low solubility in lipids (1 mg/g) (as per F01-CuASP-MO and F01-CuASP-DU), it was found that surpassing this concentration was possible because lipids could encapsulate more CuASP powder. It was in fact possible to add up to 126 mg of CuASP in 1 g of MO (as per F01-CuASP-MO-E) and up to about 130 mg in 1 g of DU (as per F01-CuASP-DU-E) (FIG. 4). The mixture remained visibly homogeneous at room temperature, however some part of the powder sedimented after centrifugation (200 rpm for 5 min). With mere mixing by spatula the formulation was again homogeneous thereby encapsulating excess CuASP in the lipid phase.

### Proposed Mechanism of Solubilization and Encapsulation

Without wishing to be bound by theory, the inventor's insights, established during the course of their inventive endeavours, led to the following mechanism of solubilisation and encapsulation being proposed.

Monoglycerides (MG), namely monoolein (MO) and monolinolein (ML) are main constituents used in our formulations. These MGs, respectively, exhibit one and two ‘cis’ unsaturations, each producing ‘kink’ at the double bond position, leading to less tight molecular packing as compared to the TGs containing saturated chains. These packing constraints affect melting points, phase behaviour and fluidity attained by lipid systems—see Kulkarni, C. V.; Tang, T. Y.; Seddon, A. M.; Seddon, J. M.; Ces, O.; Templer, R. H., Engineering Bicontinuous Cubic Structures at the Nanoscale—the Role of Chain Splay. Soft Matter 2010, 6, 3191-3194. The optimum fluidity is advantageous for the diffusion and thus the solubility of copper complexes in such systems. We have therefore utilized MG systems to develop our first formulation (F01).

Not only physical spatial organization, but also amphiphilic nature of the host system aids towards the favourable interactions between copper complex and lipid. Inverse micelles (above mp) and multiple lamellar sheets (below mp) formed from pure lipids, in contrast to the single bilayer in liposome systems, practically allow high amount of copper complex to be loaded both within the bilayers and between them. The apparent viscosity of the lipid system also encourages the copper complex particles to remain homogeneously dispersed (and thus encapsulated) in the formulation well above the solubility limit.

### Additional Examples for Copper Salicylate (CuSL)

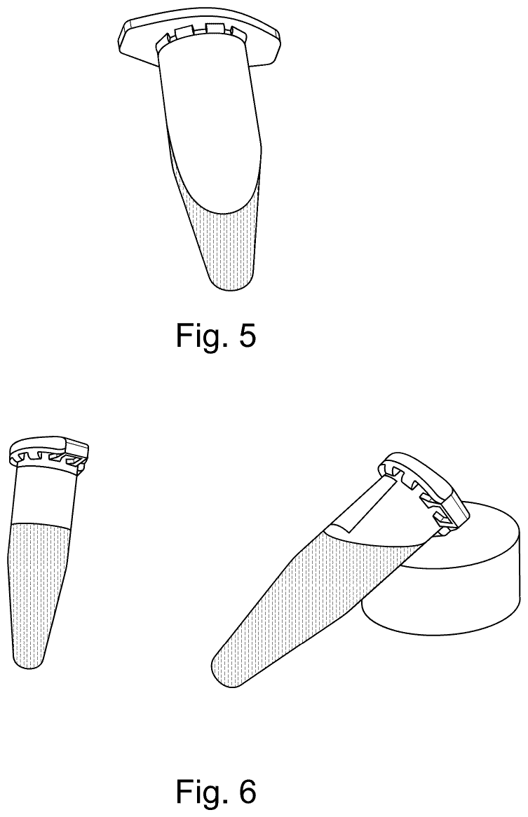

Several formulations (at varying copper salicylate concentrations) were prepared as set forth above. The grinding method (Method 2, FIG. 5) displayed better (1 mg/g of lipid) solubility as compared to the heating method (Method 1). FIG. 5 shows an image of a formulation containing CuSL solubilized in molten DU (F01-CuSL-DU).

Although CuSL has very low solubility in lipids (1 mg/g), this concentration was surpassed through lipids encapsulating more CuSL powder. It was possible to add up to 120 mg of CuSL in 1 g of MO (F01-CuSL-MO-E) and up to about 128 mg in 1 g of DU (F01-CuSL-DU-E) (FIG. 6). The mixture remained visibly homogeneous at room temperature, however some part of the powder sedimented after centrifugation (200 rpm for 5 min). With mere mixing by spatula the formulation was again homogeneous thereby encapsulating excess CuSL in the lipid phase. FIG. 6 shows two images of a formulation containing CuSL encapsulated in DU (F01-CuSL-DU-E) at room temperature (Left) and molten state (Right).

### Applications of F01-Based Formulations

The F01 formulations (with dry lipid systems) can be readily employed as a gel for topical application—this includes F01 formulations with either solubilised copper complex (CuAsp or CuSL) (as per FIG. 2) or additionally-encapsulated copper complex (as per FIG. 4).

### Further Enhancement of Copper Complex Solubility—Use of Bile Salts

Bile salts are known to interact with lipids at molecular level and facilitate phase transition in them to ease their digestion—see Sadeghpour, A.; Rappolt, M.; Misra, S.; Kulkarni, C. V., Bile Salts Caught in the Act: From Emulsification to Nanostructural Reorganization of Lipid Self-Assemblies. Langmuir 2018, 34 (45), 13626-13637. To further enhance the solubility of copper complexes in lipid and lipid-water systems, bile salt mixtures were added in various proportions. These studies are promising.

### Example 2—Copper Complex Formulations (F02): Hydrated Lipid Systems

Formulations described above (e.g. F01-CuASP-MO) can work in their current form, primarily for applications with topical or external modes of delivery. However, for internal use, an aqueous system is inevitable, which also means the CuASP loading must be studied under these conditions. We have employed F01-CuASP-MO to prepare corresponding aqueous form F02-CuASP-MO as described below.

### Preparation

Formulation F01 containing copper complex (CuASP or CuSL), prepared above, was topped up with an additional amount of said copper complex.
35-40% water (by weight) was added to the above. Spatula or similar tools were used to blend the constituents until homogeneous.
Mixtures were heated (to 40-60° C.) using block heater. Spatula or similar tools were used to blend the constituents until homogeneous.
Mixtures were heated again, if required, followed by homogenization by spatula.
Final formulation (F02, e.g. F02-CuASP-MO) was stored at room temperature (around 20° C.).

Note that, the sequence of preparation of F02 is important: first add required amount of copper complex to dry molten lipid, mix it with spatula and then add appropriate amount of water. If copper complex is added to preformed highly viscous cubic phase, it entails extra efforts (several free-thaw cycles) to obtain homogeneous formulation.

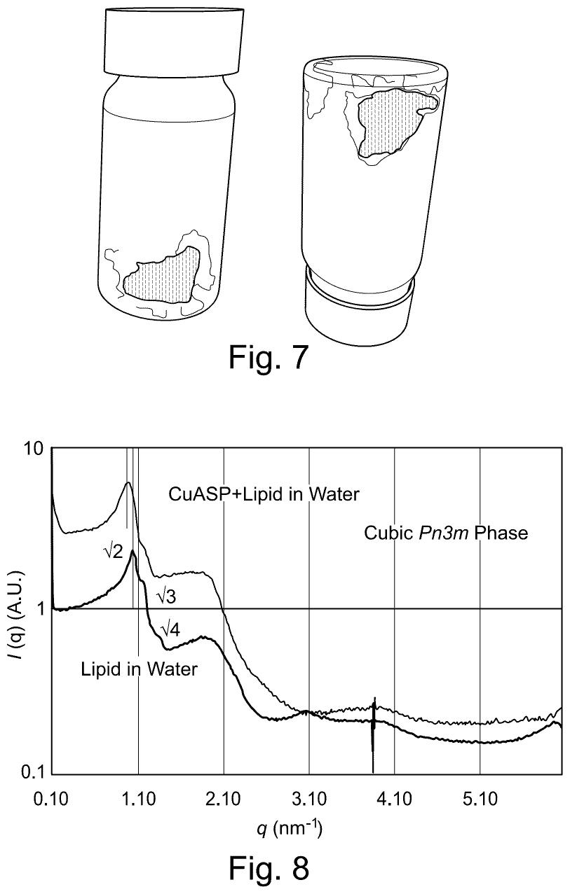

FIG. 7 shows an image of a formulation containing CuASP solubilized in molten MO+water.

FIG. 8 shows a trace showing small angle X-ray scattering patterns of pure lipid in water and CuASP loaded lipid-water mixture. Points 2, 3, 4 . . . display characteristic peaks for Pn3m cubic phase for monoolein in excess water at room temperature.

### Copper Complex Solubility for F02-MO and F02-DU

Several formulations (at varying copper aspirinate concentrations) were prepared, and the maximum solubility limits were determined using transparency check method. Briefly, 1 mm thick transparent glass slide with a small well was filled with corresponding formulation and its ability to see-through was checked, as shown by photographs referenced in Table 2.

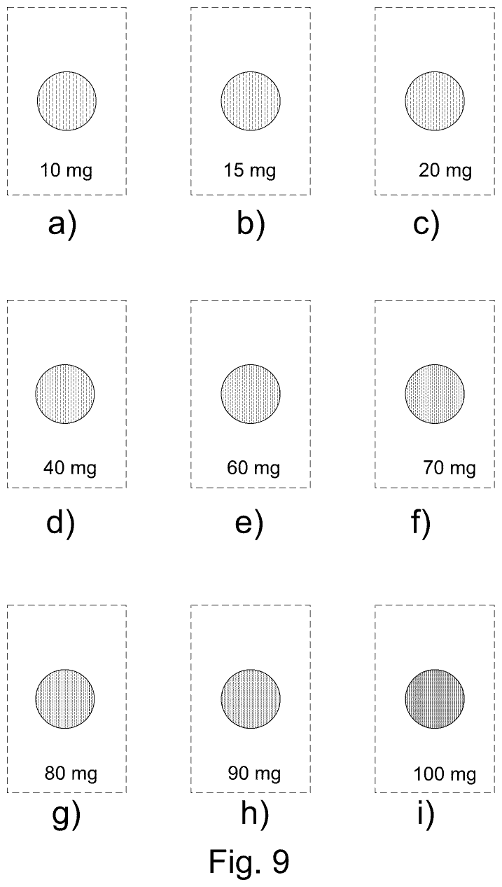

FIG. 9 shows nine images of 1 mm-thick transparent glass slides containing, within each's central well, a formulation containing 0.75 g dimodan (DU), 0.5 g water, and the following various amounts of copper(II) aspirinate: a) 10 mg; b) 15 mg; c) 20 mg; d) 40 mg; e) 60 mg; f) 70 mg; g) 80 mg; h) 90 mg; i) 100 mg.

TABLE 2
Transparency Tests for Determining Maximum Solubility of CuASP

| | | | | Transparency | |
|---|---|---|---|---|---|
| | Copper (II) | | | (From this mixture 0.1 g is | |
| | aspirinate | Dimodan | Water | transferred to a glass slide and | Photographs of |
| No. | (mg) | (g) | (g) | melted to check transparency) | glass slides |
| 1 | 10 | 0.75 | 0.50 | This is transparent, able to read the | FIG. 9a |
| | | | | letters, which means this is soluble in | |
| | | | | it. | |
| 2 | 15 | 0.75 | 0.50 | This is transparent, able to read the | FIG. 9b |
| | | | | letters, which means this is soluble in | |
| | | | | it. | |
| 3 | 20 | 0.75 | 0.50 | This is transparent, able to read the | FIG. 9c |
| | | | | letters, which means this is soluble in | |
| | | | | it. | |
| 4 | 40 | 0.75 | 0.50 | This is transparent, able to read the | FIG. 9d |
| | | | | letters, which means this is soluble in | |
| | | | | it. | |
| 5 | 60 | 0.75 | 0.50 | This is transparent, able to read the | FIG. 9e |
| | | | | letters, which means this is soluble in | |
| | | | | it. | |
| 6 | 70 | 0.75 | 0.50 | This is transparent, able to read the | FIG. 9f |
| | | | | letters, which means this is soluble in | |
| | | | | it. | |
| 7 | 80 | 0.75 | 0.50 | This is transparent, able to read the | FIG. 9g |
| | | | | letters, which means its soluble. | |
| 8 | 90 | 0.75 | 0.50 | This is not transparent, difficult to read | FIG. 9h |
| | | | | through it, which means in this mixture | |
| | | | | copper (II)aspirinate is encapsulating | |
| 9 | 100 | 0.75 | 0.50 | This is not transparent, difficult to read | FIG. 9i |
| | | | | through it, which means in this mixture | |
| | | | | copper (II)aspirinate is encapsulating | |
| 10 | 150 | 0.75 | 0.50 | copper (II)aspirinate is encapsulating | |
| 11 | 400 | 0.75 | 0.50 | copper (II)aspirinate is encapsulating | |

Several formulations (at varying copper aspirinate concentrations) were prepared and the maximum solubility number was determined to be about 80 mg CuASP in 1.25 g cubic phase (i.e. 106 mg/g of lipid)—above this concentration CuASP gets encapsulated in cubic phase (Table 2).

### Properties of Copper Complex Formulations (F02)

F02-CuASP-MO had appealing turquoise colour all throughout the sample and very high viscosity typical of lipid cubic phases (FIG. 7). Small angle X-ray scattering (SAXS) patterns confirm the presence of bicontinuous cubic Pn3m phase for MO in excess water condition at room temperature exhibiting a typical lattice parameter of 84.6 Å (FIG. 8). The latter changed detectably (to 87.9 Å) upon addition of CuASP validating the integration of CuASP in amphilic lipid phase.

### Copper Complex Encapsulation (F02-E Formulations)

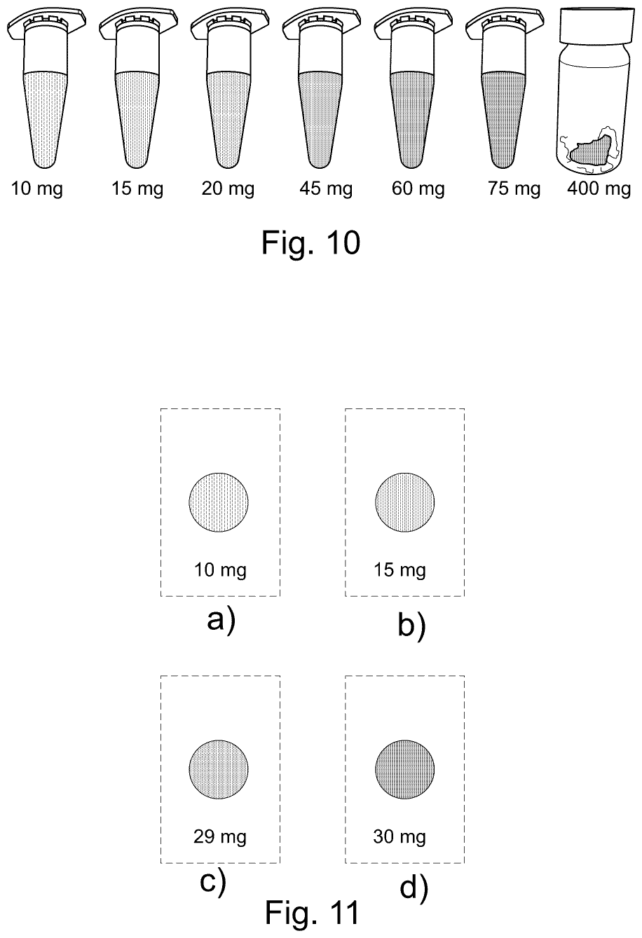

FIG. 10 shows various hydrated copper aspirinate/dimodan (DU) formulations (F02-CuASP-DU and F02-CuASP-DU-E) wherein copper aspirinate is solubilised and, for higher concentrations, is encapsulated. Characteristic turquoise colour is clearly visible from CuASP in lipid cubic phases. The numbers in mg indicate CuASP content in 1.25 g cubic phases (formed of 0.75 g lipid+0.5 g water).

About 533 mg CuASP per g of lipid (i.e. 400 mg of CuASP in 1.25 g cubic phase as shown in FIG. 10) was easily encapsulated in DU when it formed viscous cubic phase in presence of water.

### Proposed Mechanism of Solubilization and Encapsulation

Intermolecular interactions between copper complexes and lipid are perhaps similar to the F01 but water may also aid in hydrogen bonding, and therefore more copper complex is solubilized in aqueous cubic phases. Three-dimensional tortuous architecture and characteristic high viscosity of lipid cubic phase as compared to lamellar or fluid isotropic phases further helps in encapsulating very high amounts of copper complex in F02.

### Additional Examples for Copper Salicylate (CuSL)

Formulations (F02) were prepared as above using copper salicylate (CuSL) instead of copper aspirinate. The solubilization limit of CuSL: 29 mg in 1.25 g cubic phase (formed of 0.75 g lipid+0.5 g water), which equals 38 mg/g of lipid.

Several formulations (at varying copper salicylate concentrations) were prepared, and the maximum solubility limits were determined using transparency check method. Briefly, 1 mm thick transparent glass slide with a small well was filled with corresponding formulation and its ability to see-through was checked, as shown by photographs referenced in Table 3.

FIG. 11 shows four images of 1 mm-thick transparent glass slides containing, within each's central well, a formulation containing 0.75 g dimodan (DU), 0.5 g water, and the following various amounts of copper(II) salicylate: a) 10 mg; b) 15 mg; c) 29 mg; d) 30 mg.

TABLE 3
Transparency Tests for Determining Maximum Solubility of CuSL

| | | | | Transparency | |
|---|---|---|---|---|---|
| | Copper (II) | | | (From this mixture 0.1 g is transferred to | |
| | salicylate | Dimodan | Water | a glass slide and melted to check | Photographs of |
| No | (mg) | (g) | (g) | transparency) | glass slides. |
| 1. | 10 | 0.75 | 0.50 | This is transparent, able to read the | FIG. 11a |
| | | | | letters, which means this is soluble in it. | |
| 2. | 15 | 0.75 | 0.50 | This is transparent, able to read the | FIG. 11b |
| | | | | letters, which means this is soluble in it. | |
| 3. | 29 | 0.75 | 0.50 | This is transparent, able to read the | FIG. 11c |
| | | | | letters, which means this is soluble in it. | |
| 4. | 30 | 0.75 | 0.50 | This is not transparent, difficult to read | FIG. 11d |
| | | | | through it. Which means in this mixture | |
| | | | | copper (II)salicylate is encapsulating. | |
| 5. | 400 | 0.75 | 0.50 | This is not transparent, difficult to read | |
| | | | | through it. Which means in this mixture | |
| | | | | copper (II)salicylate is encapsulating. | |

About 533 mg CuSL per g of lipid (i.e. 400 mg of CuSL in 1.25 g cubic phase as shown in (FIG. 10) was easily encapsulated in DU when it formed viscous cubic phase in presence of water.

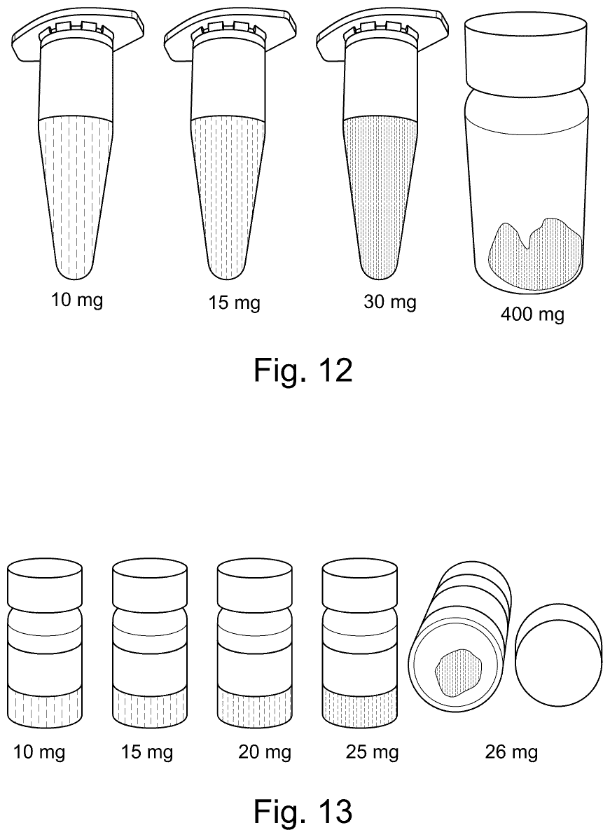

FIG. 12 shows images of various hydrated copper salicylate/dimodan (DU) formulations (F02-CuSL-DU and F02-CuSL-DU-E) wherein copper salicylate is solubilised and, for higher concentrations, is encapsulated. Characteristic green colour is clearly visible from CuSL in lipid cubic phases. The numbers in mg indicate CuASP content in 1.25 g cubic phases (formed of 0.75 g lipid+0.5 g water).

### Applications of F02-Based Formulations

The F02 formulations (with hydrated lipid systems) can be readily employed as a gel for topical application—this includes F02 formulations with either solubilised copper complex (CuAsp or CuSL) (as per Table 2, Table 3 and FIG. 7, FIG. 9, FIG. 10, FIG. 11, and FIG. 12) or additionally-encapsulated copper complex (as per Table 2, Table 3 and FIG. 9, FIG. 10, FIG. 11, and FIG. 12).

### Example 3—Copper Complex Formulations (F03): Nanostructured Dispersions/Cubosomes

Formulation F03 is in aqueous form, so it is suitable for external as well as internal modes of copper complex delivery. This formulation, however, benefits from its dispersed form as it retains the solubility properties of F02, but in addition has low viscosity making it more applicable, for instance, for an oral delivery.

These dispersions are essentially an oil-in-water (O/W) or water-in-oil (W/O) emulsions, where an oil phase contains copper complex loaded lipid or lipid+additive and water phase contains water or a stabilizer dissolved in water. The dispersions are called ‘nanostructured’ because their morphology underlays liquid crystalline lipid phase (nanostructure).

### Preparation Method for F03-DU Nanostructured Dispersions

25 mg of copper complex (CuASP or CuSL) powder was added to 0.5 g molten DU in a 20 ml vial.
It was topped with 9.5 g of pluronic F127 solution (0.5% or 1 wt % in water).
Mixture was ultra-sonicated (Sonics & Materials Vibra-Cell VCX750, Jencons, UK) for 2 to 5 minutes with 10 sec pulse and 10 sec delay using 35% of the maximum power.
Sample was warm (about 40° C.), so allowed to cool to room temperature; resulting nanostructured emulsion was homogeneous and stable without any lipid lumps.
It was stored at room temperature (around 20° C.).

FIG. 13 shows images of various oil-in-water emulsions containing CuASP/dimodan (DU). The numbers in mg indicate CuASP content in 10 g cubosomes solution (formed of 0.5 g lipid+9.5 g of 0.5% F127 solution in water). 26 mg CuASP was sedimented, so the solubility limit was reached at 25 mg CuASP in 10 g cubosomes (which is equal to 50 mg/g of lipid).

### Copper Complex Solubility for F03 Formulations

About 25 mg of CuASP was soluble in cubosomes solution of 10 g above which partial sedimentation was observed (FIG. 13). Thus, the solubility limit of CuASP in cubosomes solution was found to be 50 mg/g of lipid.

### Properties of Copper Complex Formulations (F03)

Oil-in-water emulsion i.e. cubosomes dispersion (F03-CuASP-DU) of CuASP appeared milky white homogeneous fluid and remained stable for studied 2 months (FIG. 13).

### Additional Examples for Copper Salicylate (CuSL)

Formulations (F03) were prepared as above using copper salicylate (CuSL) instead of copper aspirinate.

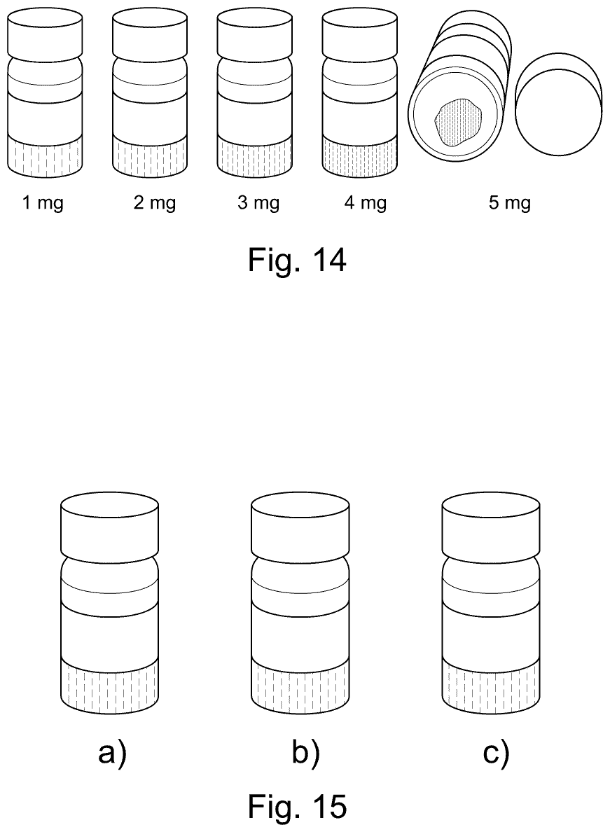

FIG. 14 shows images of various oil-in-water emulsions containing CuSL/dimodan (DU). The numbers in mg indicate CuSL content in 10 g cubosomes solution (formed of 0.5 g lipid+9.5 g of 0.5% F127 solution in water). 5 mg CuSL was sedimented, so the solubility limit was reached at 4 mg CuSL in 10 g cubosomes (which is equal to 8 mg/g of lipid).

### Further Examples for Both Copper Aspirinate (CuASP) and Copper Salicylate (CuSL)

Aqueous lipid formulations of CuASP and CuSL were further optimised.

FIG. 15 shows images of three oil-in-water emulsions containing CuASP in DU and various aqueous solutions of pluronic F127: a) 5 mg CuASP, 2 g dimodan (DU), 8 g of 2 wt % F127 solution; b) 5 mg CuASP, 4 g dimodan (DU), 6 g of 5 wt % F127 solution; c) 5 mg CuASP, 5 g dimodan (DU), 5 g of 5 wt % F127 solution.

Table 4 elucidates further the formulations of FIG. 15.

TABLE 4
Details of CuASP Emulsions with DU in aqueous F127 solutions (FIG. 15)

| | | | Pluronic F-127 | | |
|---|---|---|---|---|---|
| | | | (g) | | |
| | | | Wt of | (%) | |
| SI. | Copper (II) | Dimodian | F127 aq | F127 | |
| No | Aspirinate (mg) | (g) | solution | wt % | Observations |
| a | 5 | 2 | 8 | 2 | A very viscous emulsion formed, no |
| | | | | | sedimentation. |
| b | 5 | 4 | 6 | 5 | A gel was formed with less white dots (white |
| | | | | | dot is due to the crystallization of Dimodan). |
| c | 5 | 5 | 5 | 5 | A gel formed with white dots. |

From FIG. 15 and Table 4 it is apparent that increasing the Dimodan (DU) concentration permits more dissolution of the copper (II) aspirinate.

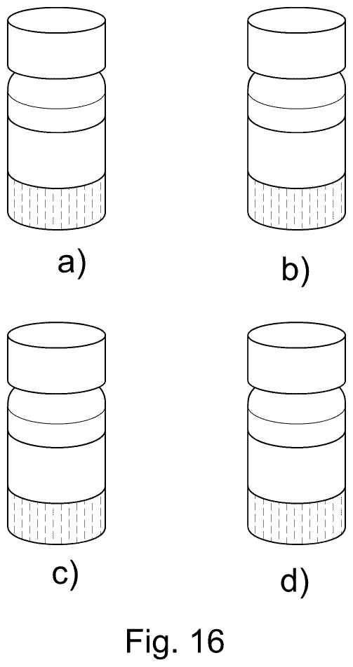

It was also possible to form CuASP emulsions with DU, aqueous F127 solution, and coconut oil, as detailed in Table 5 and shown in FIG. 16.

FIG. 16 shows images of four oil-in-water emulsion containing CuASP in DU+coconut oil and various aqueous solutions of pluronic F127: a) 5 mg CuASP, 0.9 g dimodan (DU), 0.1 g coconut oil, 9 g of 0.5 wt % F127 solution; b) 5 mg CuASP, 1.8 g dimodan (DU), 0.2 g coconut oil, 8 g of 0.5 wt % F127 solution; c) 5 mg CuASP, 3.6 g dimodan (DU), 0.4 g coconut oil, 6 g of 1 wt % F127 solution; a) 5 mg CuASP, 4.5 g dimodan (DU), 0.5 g coconut oil, 5 g of 2 wt % F127 solution.

TABLE 5
Details of CuASP Emulsions with DU and Coconut
Oil in aqueous F127 solutions (FIG. 16)

| | | | | Pluronic F-127 | | |
|---|---|---|---|---|---|---|
| | | | | (g) | | |
| | | | | Wt of | (%) | |
| SI. | Copper (II) | Dimodian | Coconut | F127 aq | F127 | |
| No | Aspirinate (mg) | (g) | oil (g) | solution | wt % | Observations |
| a. | 5 | 0.9 | 0.1 | 9 | 0.5 | An emulsion formed, but a |
| | | | | | | small amount of copper (II) |
| | | | | | | aspirinate was sedimented in |
| | | | | | | the bottom. |
| b. | 5 | 1.8 | 0.2 | 8 | 0.5 | An emulsion formed, slightly |
| | | | | | | viscous than the above. No |
| | | | | | | sedimentation. |
| c. | 5 | 3.6 | 0.4 | 6 | 1 | A viscous cream was formed, |
| | | | | | | no sedimentation. |
| d. | 5 | 4.5 | 0.5 | 5 | 2 | A cream formed, more |
| | | | | | | viscous than the above. |

NB: The proportion of components for 90:10 DU coconut oil formulation, i.e. formulation a in Table 5 were as follows: a=0.05%, b1=9%, b2=1%, c=89.5%, d=0.45%, where, a=copper complex, b1=lipid, b2=coconut oil, c=water, d=surfactant stabilizer.

The above examples with copper aspirinate can also be replicated for another copper complex, namely copper salicylate (CuSL).

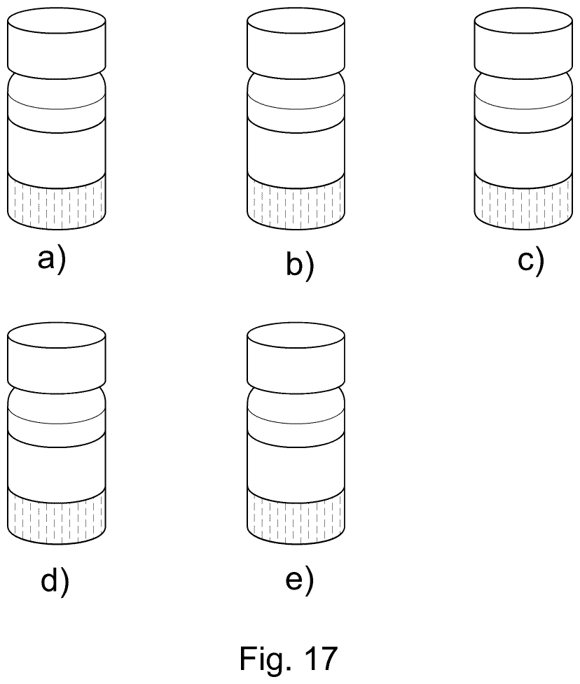

FIG. 17 shows images of five oil-in-water emulsions containing CuSL in DU and various aqueous solutions of pluronic F127: a) 5 mg CuSL, 0.5 g dimodan (DU), 9.5 g of 0.5 wt % F127 solution; b) 5 mg CuSL, 1 g dimodan (DU), 9 g of 1 wt % F127 solution; c) 5 mg CuSL, 2 g dimodan (DU), 8 g of 2 wt % F127 solution; d) 5 mg CuSL, 4 g dimodan (DU), 6 g of 5 wt % F127 solution; e) 5 mg CuSL, 5 g dimodan (DU), 5 g of 5 wt % F127 solution.

Table 6 further elucidates the formulations shown in FIG. 17.

TABLE 6
Details of CuSL Emulsions with DU in aqueous F127 solutions (FIG. 17)

| | | | Pluronic F-127 | | |
|---|---|---|---|---|---|
| | | | (g) | | |
| | | | Wt of | (%) | |
| SI. | Copper (II) | Dimodian | F127 aq | F127 | |
| No | Salicylate (mg) | (g) | solution | wt % | Observations |
| a. | 5 | 0.5 | 9.5 | 0.5 | An emulsion formed, no water and oil |
| | | | | | separation but a small amount of copper |
| | | | | | (II) salicylate was sedimented in the |
| | | | | | bottom. |
| b. | 5 | 1 | 9 | 1 | An emulsion formed, slightly viscous than |
| | | | | | the above. The sedimentation was very |
| | | | | | less compared to the above. |
| c. | 5 | 2 | 8 | 2 | A very viscous emulsion formed, no |
| | | | | | sedimentation. |
| d. | 5 | 4 | 6 | 5 | A gel was formed with less white dots |
| | | | | | (white dot is due to the crystallization of |
| | | | | | Dimodan). |
| e. | 5 | 5 | 5 | 5 | A gel formed with white dots. |

It is apparent from Table 6 and FIG. 17 that increasing the Dimodian concentration permits more copper (II) salicylate to be dissolved.

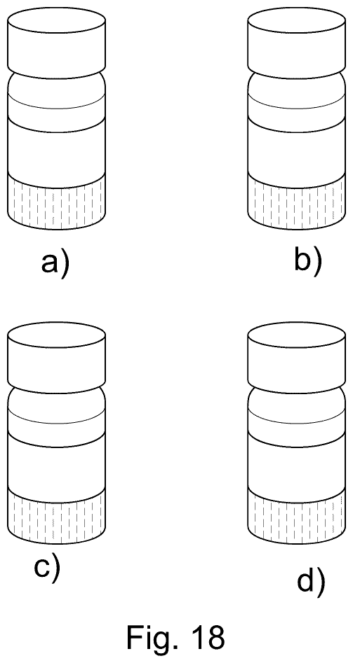

It was also possible to form CuSL emulsions with DU, aqueous F127 solution, and coconut oil, as detailed in Table 7 and shown in FIG. 18.

FIG. 18 shows images of four oil-in-water emulsion containing CuSL in DU+coconut oil and various aqueous solutions of pluronic F127: a) 5 mg CuSL, 0.9 g dimodan (DU), 0.1 g coconut oil, 9 g of 0.5 wt % F127 solution; b) 5 mg CuSL, 1.8 g dimodan (DU), 0.2 g coconut oil, 8 g of 0.5 wt % F27 solution; c) 5 mg CuSL, 3.6 g dimodan (DU), 0.4 g coconut oil, 6 g of 1 wt % F127 solution; a) 5 mg CuSL, 4.5 g dimodan (DU), 0.5 g coconut oil, g of 2 wt % F127 solution.

TABLE 7
Details of CuSL Emulsions with DU and Coconut
Oil in aqueous F127 solutions (FIG. 18)

| | | | | Pluronic F-127 | | |
|---|---|---|---|---|---|---|
| | | | | (g) | | |
| | | | | Wt of | (%) | |
| SI. | Copper (II) | Dimodian | Coconut | F127 aq | F127 | |
| No | salicylate (mg) | (g) | oil (g) | solution | wt % | Observations |
| a. | 5 | 0.9 | 0.1 | 9 | 0.5 | An emulsion formed, but a |
| | | | | | | small amount of copper (II) |
| | | | | | | salicylate was sedimented in |
| | | | | | | the bottom. |
| b. | 5 | 1.8 | 0.2 | 8 | 0.5 | An emulsion formed, slightly |
| | | | | | | viscous than the above. No |
| | | | | | | sedimentation. |
| c. | 5 | 3.6 | 0.4 | 6 | 1 | A viscous cream was formed, |
| | | | | | | no sedimentation. |
| d. | 5 | 4.5 | 0.5 | 5 | 2 | A cream formed, more viscous |
| | | | | | | than the above. |

### Applications of F03-Based Formulations

The F03 formulations (aqueous dispersions/emulsions/cubosomes) can be readily employed as a fluid oral dosage form (e.g. FIG. 13, FIG. 14, FIG. 15, FIG. 16, FIG. 17, and FIG. 18).

### Numbered Paragraphs

The present invention, and embodiments thereof, may also be defined by way of the following numbered paragraphs.
1. A composition comprising:
   a copper-drug complex (or salt thereof) comprising one or more copper ion(s), and one or more ligand(s) datively bonded to at least one of the copper ion(s), wherein at least one of the ligand(s) is a drug molecule (or ionised form thereof); and
   one or more glycerides, wherein at least one of the glyceride(s) comprises at least one unsaturated moiety, preferably at least one alkene moiety, most preferably at least one cis-alkene moiety.
2. The composition as defined in paragraph 1, wherein the one or more glycerides are one or more monoglycerides bearing a C₈-C₂₀ fatty acid moiety having at least one cis-alkene.
3. The composition as defined in paragraph 2, wherein the one or more glycerides are a first monoglyceride and a second monoglyceride, each bearing a C₈-C₂₀ fatty acid moiety having at least one cis-alkene.
4. The composition as defined in paragraph 3, wherein the one or more glycerides are glyceryl monooleate and glyceryl monolinoleate.
5. The composition as defined in paragraph 4, wherein the glyceryl monooleate and glyceryl monolinoleate are present in a weight ratio between 10:90 and 90:10.
6. The composition as defined in any preceding paragraph, wherein the copper-drug complex is a copper(II)-drug complex such that the one or more copper ion(s) are one or more copper(II) ion(s).
7. The composition as defined in any preceding paragraph, wherein the drug molecule is defined by Formula I:

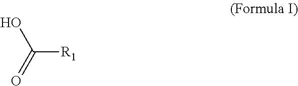

wherein R₁ is an optionally substituted organic (i.e. carbon-containing) group suitably which together with the carboxylic acid moiety completes the drug molecule; or an ionised form thereof.
8. The composition as defined in paragraph 7, wherein the drug molecule is defined by Formula III:

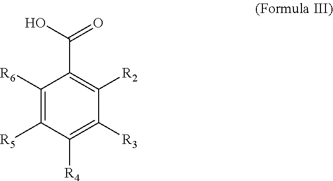

wherein each of R₂, R₃, R₄, R₅, and R₆ are independently either hydrogen or an Rₓ group;
wherein each Rₓ group is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

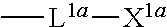

wherein:
   L^{1a} is absent or is selected from O, S, SO, SO₂, N(Rₐ), CO, C(O)O, C(O)O, OC(O)O, CH(ORₐ), CON(Rₐ), N(Rₐ)CO, N(Rₐ)CON(R_{b}), SO₂N(Rₐ), N(Rₐ)SO₂, OC(Rₐ)₂, OC(Rₐ)₂O, SC(Rₐ)₂ and N(Rₐ)C(R_{b})₂, where Rₐ and R_{b}, which may be the same or different, are each independently selected from hydrogen or any Rₓ group; and
   X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
   wherein any Rₓ group is optionally further substituted with one or more Rₓ groups as defined above or any ionised form thereof.
9. The composition as defined in paragraph 8, wherein each Rₓ group is independently selected from hydroxy, (1-6C)alkoxy, and (2-6C)alkanoyloxy; wherein most suitably each Rₓ group is independently selected from hydroxy and acetoxy).
10. The composition as defined in any preceding paragraph, wherein the copper-drug complex is copper(II) aspirinate (or a salt thereof) or copper(II) salicylate (or a salt thereof).
11. The composition as defined in any preceding paragraph, wherein the copper-drug complex is copper(II) aspirinate.
12. The composition as defined in any preceding paragraph, wherein the composition comprises 0.001-40 wt % copper-drug complex.
13. The composition as defined in paragraph 12, wherein the weight ratio of the copper-drug complex to the glyceride(s) is 0.1:1000-700:1000.
14. The composition as defined in any preceding paragraph, wherein the composition comprises 0.005-30 wt % copper-drug complex, 1-99.99 wt % glyceride(s), and optionally 0-99 wt % water.
15. The composition as defined in any of paragraphs 1 to 13, wherein the composition comprises 2-20 wt % copper-drug complex, 50-95 wt % glyceride(s), and at most 2 wt % water, wherein the weight ratio of the copper-drug complex to the glyceride(s) is 10:1000-400:1000.
16. The composition as defined in any of paragraphs 1 to 13, wherein the composition comprises 0.1-15 wt % copper-drug complex, 30-90 wt % glyceride(s), and 15-60 wt % water, wherein the weight ratio of the copper-drug complex to the glyceride(s) is 1:1000-200:1000.
17. The composition as defined in any of paragraphs 1 to 13, wherein the composition comprises 5-40 wt % copper-drug complex, 20-80 wt % glyceride(s), and 10-50 wt % water, wherein the weight ratio of the copper-drug complex to the glyceride(s) is 50:1000-700:1000.
18. The composition as defined in any of paragraphs 1 to 13, wherein the composition comprises 0.01-1 wt % copper-drug complex, 0.1-20 wt % glyceride(s), and 45-99 wt % water, wherein the weight ratio of the copper-drug complex to the glyceride(s) is 1:1000-200:1000.
19. The composition as defined in paragraph 18, wherein the composition comprises 0.01-2 wt % surfactant(s).
20. The composition as defined in paragraph 19, wherein the surfactant(s) is/are selected a non-ionic surfactant(s) selected form the group consisting of a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, polyglycerol esters, sucrose esters, saponins (e.g. saponin), and any combination thereof.
21. The composition as defined in paragraph 20, wherein the surfactant is poloxamer 407 (e.g. Pluronic F127).
22. The composition as defined in any preceding paragraph, wherein the stipulated glyceride(s) constitute at least 85 wt % of the total amount of lipids present within the composition.
23. The composition as defined in any preceding paragraph, wherein at least 50 wt % of the composition is constituted by the stipulated ingredients (copper-drug complex, glyceride(s), any water that is present, and any additionally stipulated ingredients).
24. The composition as defined in any preceding paragraph, wherein centrifugation (e.g. 3000 rpm for 5 min at RT) of the composition yields less than 2 wt % sedimentation (i.e. less than 2 wt % of the composition as a whole).
25. The composition as defined in any preceding paragraph, wherein the composition comprises bile salts (and/or bile acid(s)).
26. The composition as defined in any preceding paragraph, wherein the composition comprises one or more oils.
27. The composition as defined in any preceding paragraph, wherein the weight ratio of unstipulated lipid(s) (i.e. lipid(s) other than the one or more glyceride(s) stipulated in any preceding paragraph) to stipulated glyceride(s) (i.e. the one or more glycerides stipulated in any preceding paragraph) is between 0:100 to 15:85 (i.e. the weight ratio is less than or equal to 15:85).
28. The composition as defined in any preceding paragraph, wherein the composition is a topical composition.
29. The composition as defined in paragraph 28, wherein topical composition is a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.
30. The composition as defined in any of paragraphs 1 to 27, wherein the composition is an ingestible composition.
31. The composition as defined in paragraph 30, wherein the ingestible composition is (or is incorporated within a product which is) selected from a lozenge, a gel, a jelly, a pastille, a tablet, a capsule (e.g. a capsule containing the composition), a toffee, a nougat, a chewy candy, and/or a chewing gum.
32. The composition as defined in paragraph 30, wherein the ingestible composition is an aqueous emulsion, aqueous dispersion, aqueous suspension, or aqueous solution.
33. A composition as defined in any preceding paragraph for use as a medicament.
34. A composition as defined in any of paragraphs 1 to 32 for use as an antiulcer, anticancer, antimutagenic, antithrombotic, antifungal, antimicrobial, and antiviral treatment, and/or for use in the treatment of rheumatoid arthritis and/or similar disorders (e.g. psoriasis, psoriatic arthritis, ankylosing spondylitis, juvenile idiopathic arthritis, Crohn's disease, Ulcerative colitis, and Uveitis).
35. A composition as defined in any of paragraphs 1 to 32 for use in the treatment of ulcers, cancers, thromboses, fungal infections microbial infections, viral infections, rheumatoid arthritis and/or similar disorders (e.g. psoriasis, psoriatic arthritis, ankylosing spondylitis, juvenile idiopathic arthritis, Crohn's disease, Ulcerative colitis, and Uveitis).

1. A composition comprising:
a copper-drug complex (or salt thereof) comprising one or more copper ion(s), and one or more ligand(s) datively bonded to at least one of the copper ion(s), wherein at least one of the ligand(s) is a drug molecule (or ionised form thereof); and
one or more monoglycerides, wherein at least one of the monoglyceride(s) comprises at least one unsaturated moiety, preferably at least one alkene moiety, most preferably at least one cis-alkene moiety.

2. The composition as claimed in claim 1, wherein the one or more monoglycerides are one or more monoglycerides bearing a C8-C20 fatty acid moiety having at least one cis-alkene.

3. The composition as claimed in claim 2, wherein the one or more monoglycerides are a first monoglyceride and a second monoglyceride, each bearing a C8-C20 fatty acid moiety having at least one cis-alkene.

4. The composition as claimed in claim 3, wherein the one or more monoglycerides are glyceryl monooleate and glyceryl monolinoleate.

5. The composition as claimed in claim 4, wherein the glyceryl monooleate and glyceryl monolinoleate are present in a weight ratio between 10:90 and 90:10.

6. The composition as claimed in claim 1, wherein the copper-drug complex is a copper(II)-drug complex such that the one or more copper ion(s) are one or more copper(II) ion(s).

7. The composition as claimed in claim 1, wherein the drug molecule is defined by Formula I: 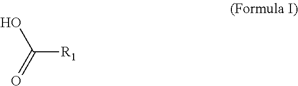
wherein R₁ is an optionally substituted organic (i.e. carbon-containing) group suitably which together with the carboxylic acid moiety completes the drug molecule;
or an ionized form thereof.

8. The composition as claimed in claim 7, wherein the drug molecule is defined by Formula III: 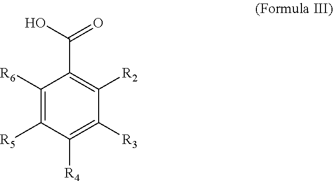
wherein each of R₂, R₃, R₄, R₅, and R₆ are independently either hydrogen or an Rₓ group;
wherein each Rₓ group is independently selected from halogeno, trifluoromethyl, cyano, isocyano, nitro, hydroxy, mercapto, amino, formyl, carboxy, carbamoyl, ureido, (1-8C)alkyl, (2-8C)alkenyl, (2-8C)alkynyl, (1-8C)hydroxyalkyl, (1-6C)alkoxy, (1-6C)alkylamino, (1-6C)dialkylamino, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N′-(1-6C)alkylureido, N′,N′-di-[(1-6C)alkyl]ureido, N-(1-6C)alkylureido, N,N′-di-[(1-6C)alkyl]ureido, N,N′,N′-tri-[(1-6C)alkyl]ureido,N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula: 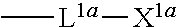
wherein:
L^{1a} is absent or is selected from O, S, SO, SO₂, N(Rₐ), CO, C(O)O, C(O)O, OC(O)O, CH(ORa), CON(Rₐ), N(Rₐ)CO, N(Rₐ)CON(Rb), SO₂N(Rₐ), N(Rₐ)SO₂, OC(Rₐ)₂, OC(Rₐ)₂O, SC(Rₐ)₂ and N(Rₐ)C(Rb)₂, where Rₐ and R_{b}, which may be the same or different, are each independently selected from hydrogen or any Rₓ group; and
X^{1a} is aryl, aryl-(1-6C)alkyl, (3-8C)cycloalkyl, (3-8C)cycloalkyl-(1-6C)alkyl, (3-8C)cycloalkenyl, (3-8C)cycloalkenyl-(1-6C)alkyl, heteroaryl, heteroaryl-(1-6C)alkyl, heterocyclyl or heterocyclyl-(1-6C)alkyl;
wherein any Rₓ group is optionally further substituted with one or more Rₓ groups as defined above
or any ionized form thereof.

9. The composition as claimed in claim 8, wherein each Rₓ group is independently selected from hydroxy, (1-6C)alkoxy, and (2-6C)alkanoyloxy; wherein most suitably each Rₓ group is independently selected from hydroxy and acetoxy).

10. The composition as claimed in claim 1, wherein the copper-drug complex is copper(II) aspirinate (or a salt thereof) or copper(II) salicylate (or a salt thereof).

11. The composition as claimed in claim 1, wherein the copper-drug complex is copper(II) aspirinate.

12. The composition as claimed in claim 1, wherein the composition comprises 0.001-40 wt % copper-drug complex.

13. The composition as claimed in claim 12, wherein the weight ratio of the copper-drug complex to the monoglyceride(s) is 0.1:1000-700:1000.

14. The composition as claimed in claim 1, wherein the composition comprises 0.005-30 wt % copper-drug complex, 1-99.99 wt % monoglyceride(s), and optionally 0-99 wt % water.

15. The composition as claimed in claim 1, wherein the composition comprises 2-20 wt % copper-drug complex, 50-95 wt % monoglyceride(s), and at most 2 wt % water, wherein the weight ratio of the copper-drug complex to the glyceride(s) is 10:1000-400:1000.

16. The composition as claimed in claim 1, wherein the composition comprises 0.1-15 wt % copper-drug complex, 30-90 wt % monoglyceride(s), and 15-60 wt % water, wherein the weight ratio of the copper-drug complex to the glyceride(s) is 1:1000-200:1000.

17. The composition as claimed in claim 1, wherein the composition comprises 5-40 wt % copper-drug complex, 20-80 wt % monoglyceride(s), and 10-50 wt % water, wherein the weight ratio of the copper-drug complex to the glyceride(s) is 50:1000-700:1000.

18. The composition as claimed in claim 1, wherein the composition comprises 0.01-1 wt % copper-drug complex, 0.1-20 wt % monoglyceride(s), and 45-99 wt % water, wherein the weight ratio of the copper-drug complex to the glyceride(s) is 1:1000-200:1000.

19. The composition as claimed in claim 18, wherein the composition comprises 0.01-2 wt % surfactant(s).

20. The composition as claimed in claim 19, wherein the surfactant(s) is/are selected a non-ionic surfactant(s) selected form the group consisting of a fatty alcohol, a fatty alcohol ether, a fatty acid ester, a fatty acid amide, a polyoxyalkylene alkyl ether, a polyoxyethylene alkyl ether, a non-ionic block copolymer, alpha-tocopherol, polyglycerol esters, sucrose esters, saponins (e.g. saponin), and any combination thereof.

21. The composition as claimed in claim 20, wherein the surfactant is poloxamer 407 (e.g. Pluronic F127).

22. The composition as claimed in claim 1, wherein the stipulated monoglyceride(s) constitute at least 85 wt % of the total amount of lipids present within the composition.

23. The composition as claimed in claim 1, wherein at least 50 wt % of the composition is constituted by the stipulated ingredients (copper-drug complex, monoglyceride(s), any water that is present, and any additionally stipulated ingredients).

24. The composition as claimed in claim 1, wherein centrifugation (e.g. 3000 rpm for 5 min at RT) of the composition yields less than 2 wt % sedimentation (i.e. less than 2 wt % of the composition as a whole).

25. The composition as claimed in claim 1, wherein the composition comprises bile salts (and/or bile acid(s)).

26. The composition as claimed in claim 1, wherein the composition comprises one or more oils.

27. The composition as claimed in claim 1, wherein the weight ratio of unstipulated lipid(s) (i.e. lipid(s) other than the one or more monoglyceride(s) stipulated in any preceding claim) to stipulated monoglyceride(s) (i.e. the one or more monoglycerides stipulated in any preceding claim) is between 0:100 to 15:85 (i.e. the weight ratio is less than or equal to 15:85).

28. The composition as claimed in claim 1, wherein the composition is a topical composition.

29. The composition as claimed in claim 28, wherein topical composition is a cream, gel formulation, foam, ointment, spray, perfume (e.g. perfume, aftershave, cologne, or eau de toilette), salve, and/or film, suitably which is intended to be applied to the skin or body cavity and are not intended to be taken by mouth, most preferably a topical composition that is a cream or ointment.

30. The composition as claimed in claim 1, wherein the composition is an ingestible composition.

31. The composition as claimed in claim 30, wherein the ingestible composition is (or is incorporated within a product which is) selected from a lozenge, a gel, a jelly, a pastille, a tablet, a capsule (e.g. a capsule containing the composition), a toffee, a nougat, a chewy candy, and/or a chewing gum.

32. The composition as claimed in claim 30, wherein the ingestible composition is an aqueous emulsion, aqueous dispersion, aqueous suspension, or aqueous solution.

33-35. (canceled)

36. A composition comprising:
a metal compound and/or metal complex (or salt thereof); and
one or more monoglycerides, wherein at least one of the monoglyceride(s) comprises at least one unsaturated moiety, preferably at least one alkene moiety, most preferably at least one cis-alkene moiety.

37. The composition as claimed in claim 36, wherein the metal compound and/or metal complex (or salt thereof) is an antimicrobial and/or antifungal metal compound and/or metal complex (or salt thereof).

38. The composition as claimed in claim 36, wherein the composition comprises a metal compound.

39. The composition as claimed in claim 38, wherein the metal compound is a copper compound, suitably a copper compound selected from the group consisting of a copper oxide, a copper halide (e.g. copper fluoride, copper chloride, copper bromide, copper iodide), a copper nitrate, a copper nitrite, a copper sulphate, a copper phosphate, and any combination thereof.

40. The composition as claimed in claim 39, wherein the composition comprises:
a copper compound, suitably a copper compound selected from the group consisting of a copper oxide, a copper halide (e.g. copper fluoride, copper chloride, copper bromide, copper iodide), a copper nitrate, a copper nitrite, a copper sulphate, a copper phosphate, and any combination thereof; and
glyceryl monooleate and glyceryl monolinoleate.

41. The composition as claimed in claim 40, wherein the weight ratio of the copper compound to the combined glyceryl monooleate and glyceryl monolinoleate is 0.1:1000-700:1000.

42. A method for treating a condition treatable by metal complexes, comprising administering to a subject in need thereof a therapeutically effective amount of the composition as claimed in claim 1.

43. The method as claimed in claim 42, wherein the condition is an ulcer, cancer, thrombosis, fungal infection, microbial infection, viral infection, rheumatoid arthritis, psoriasis, psoriatic arthritis, ankylosing spondylitis, juvenile idiopathic arthritis, Crohn's disease, Ulcerative colitis, or Uveitis).
